(19) United States
(12) Patent Application Publication
(10) Pub. No.: US 2012/0028988 A1
(43) Pub. Date: Feb. 2, 2012

(54) USE OF PYRIDAZINONE COMPOUND FOR CONTROL OF HARMFUL ARTHROPOD PESTS

(75) Inventors: Norihisa Sakamoto, Sanda-shi (JP); Takafumi Fusaka, Kobe-shi (JP)

(73) Assignee: SUMITOMO CHEMICAL COMPANY, LIMITED, Tokyo (JP)

(21) Appl. No.: 13/259,864

(22) PCT Filed: Mar. 24, 2010
(86) PCT No.: PCT/JP2010/055772

§ 371 Date: Sep. 28, 2011

(30) Foreign Application Priority Data
Mar. 30, 2009 (JP) 2009-081638
Jul. 17, 2009 (JP) 2009-168720
Oct. 7, 2010 (WO) 2010/113986

(51) Int. Cl. A01N 43/58; C07D 401/10; A01P 7/00; C07D 237/16

(52) U.S. Cl. 514/252.03; 544/240; 544/238; 514/247; 514/252.01

(57) ABSTRACT

Since a pyridazinone compound shown by formula (I) has an activity for controlling arthropod pests, the pyridazinone compound is effective for a use for controlling arthropod pests.

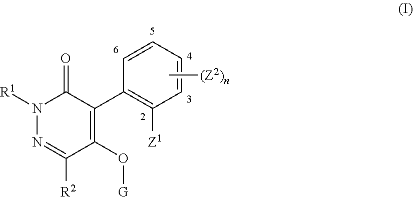

### TECHNICAL FIELD

The present invention relates to a use of a pyridazinone compound for controlling arthropod pests.

### BACKGROUND ART

WO2007/119434 and WO2009/035150 describe some sorts of pyridazinone compounds.

### DISCLOSURE OF THE INVENTION

However, it is not known that these pyridazinone compounds can be used for a use for controlling arthropod pest.

The present invention is based on that these pyridazinone compounds have control effects on arthropod pests. More specifically, the present invention provides a use of a pyridazinone compound for controlling arthropod pests.

The present invention is as follows.

[1] An agent for controlling arthropod pests (hereinafter, referred to as the control agent of the present invention) comprising a pyridazinone compound shown by formula (I) (hereinafter, sometimes referred to as the present compound):

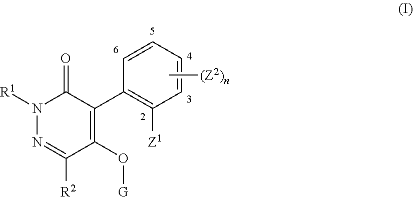

[wherein R² represents a C₁₋₆ alkyl group or a (C₁₋₆ alkyloxy) C₁₋₆ alkyl group, R² represents hydrogen or a C₁₋₆ alkyl group, G represents hydrogen, a group represented by:

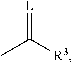

a group represented by:

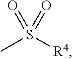

or a group represented by:

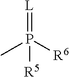

{wherein L represents oxygen or sulfur,

R³ represents a C₂₋₆ alkyl group, a C₃₋₈ cycloalkyl group, a C₂₋₆ alkenyl group, a C₂₋₆ alkynyl group, a C₆₋₂₀ aryl group, a (C₆₋₁₀ aryl) C₁₋₆ alkyl group, a C₂₋₆ alkyloxy group, a C₃₋₈ cycloalkyloxy group, a C₂₋₆ alkenyloxy group, a C₃₋₆ alkynyloxy group, a C₆₋₂₀ aryloxy group, a (C₆₋₂₀ aryl) C₁₋₆ alkyloxy group, a di (C₁₋₆ alkyl)amino group, a di (C₂₋₆ alkenyl)amino group, a (C₁₋₆ alkyl) (C₆₋₁₀ aryl)amino group or a 3- to 8-membered nitrogen-containing heterocyclic group,

R⁴ represents a C₂₋₆ alkyl group, a C₆₋₂₀ aryl group or a di (C₂₋₆ alkyl)amino group, and

R⁵ and R⁶ may be the same or different, and represent a C₂₋₆ alkyl group, a C₃₋₈ cycloalkyl group, a C₂₋₆ alkenyl group, a C₆₋₂₀ aryl group, a C₂₋₆ alkyloxy group, a C₃₋₈ cycloalkyloxy group, a C₆₋₂₀ aryloxy group, a (C₆₋₁₀ aryl) C₁₋₆ alkyloxy group, a C₂₋₆ alkylthio group or a di (C₂₋₆ alkyl)amino group,

wherein any group each represented by R³, R⁴, R⁵ and R⁶ may be substituted with halogen, and any of the C₃₋₈ cycloalkyl group, the C₆₋₂₀ aryl group, the aryl moiety of the (C₆₋₁₀ aryl) C₁₋₆ alkyl group, the C₃₋₈ cycloalkyloxy group, the C₆₋₂₀ aryloxy group, the aryl moiety of the (C₆₋₁₀ aryl) C₁₋₆ alkyloxy group, the aryl moiety of the (C₁₋₆ alkyl) (C₆₋₁₀ aryl)amino group and the 3- to 8-membered nitrogen-containing heterocyclic group may be substituted with a C₁₋₆ alkyl group},

Z¹ represents a C₁₋₆ alkyl group,

Z² represents a C₁₋₆ alkyl group, a C₃₋₈ cycloalkyl group, a C₂₋₆ alkynyl group, a C₁₋₆ haloalkyl group, a C₆₋₁₀ aryl group, a 5- or 6-membered heteroaryl group, a C₁₋₆ alkyloxy group, a C₁₋₆ haloalkyloxy group, halogen, a cyano group or a nitro group, wherein the C₃₋₈ cycloalkyl group, the C₆₋₁₀ aryl group and the 5- or 6-membered heteroaryl group represented by Z² may be substituted with at least one group selected from the group consisting of halogens and C₁₋₆ alkyl groups, and

n represents 0, 1, 2, 3, or 4, and when n is 2, 3, or 4, each of Z² may be the same or different], and

an inert carrier.

[2] The agent for controlling arthropod pests according to [1], wherein, in the formula (I), n is 1, 2, or 3.

[3] The agent for controlling arthropod pests according to [1], wherein, in the formula (I), n is 0, and Z¹ is a C₂₋₆ alkyl group.

[4] The agent for controlling arthropod pests according to [1] or [2], wherein, in the formula (I), n is 1 or 2, and the binding position of Z² is a 4- and/or 6-positions on the benzene ring.

[5] The agent for controlling arthropod pests according to any one of [1], [2], and [4], wherein, in the formula (I), Z¹ is a C₁₋₃ alkyl group, and Z² is a C₁₋₃ alkyl group, a C₃₋₆ cycloalkyl group, a C₂₋₃ alkynyl group, a C₁₋₃ alkyloxy group, halogen, a cyano group or a nitro group, or a phenyl group which may be substituted with at least one group selected from the group consisting of halogens and C₁₋₃ alkyl groups.

[6] The agent for controlling arthropod pests according to any one of [1], [2], [4], and [5], wherein, in the formula (I), Z¹ is a C₁₋₃ alkyl group, and Z² is a C₁₋₃ alkyl group.

[7] The agent for controlling arthropod pests according to any one of [1] and [2], wherein, in the formula (I), n is 1, and the binding position of Z² is a 5-position on the benzene ring.

[8] The agent for controlling arthropod pests according to any one of [1], [2], and [7], wherein, in the formula (I), n is 1, the binding position of Z² is a 5-position on the benzene ring, Z² is a C₁₋₃ alkyl group, and Z² is a phenyl group which may be substituted with at least one group selected from the group consisting of halogens and C₁₋₃ alkyl groups.

[9] The agent for controlling arthropod pests according to any one of [1] to [8], wherein, in the formula (I), R² is a C₁₋₃ alkyl group or a (C₁₋₃ alkyloxy)C₁₋₃ alkyl group.

[10] The agent for controlling arthropod pests according to any one of [1] to [9], wherein, in the formula (I), R² is a methyl group.

[11] The agent for controlling arthropod pests according to any one of [1] to [10], wherein, in the formula (I), R² is hydrogen or a C₁₋₃ alkyl group.

[12] The agent for controlling arthropod pests according to any one of [1] to [11], wherein, in the formula (I), R² is hydrogen or a methyl group.

[13] The agent for controlling arthropod pests according to any one of [1] to [12], wherein, in the formula (I), G is hydrogen, a group represented by:

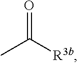

a group represented by:

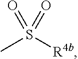

or a group represented by:

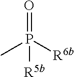

[wherein R^{3b} represents a C₁₋₆ alkyl group, a C₃₋₈ cycloalkyl group, a C₂₋₆ alkenyl group, a C₂₋₆ alkynyl group, a C₆₋₁₀ aryl group, a (C₆₋₁₀ aryl) C₁₋₆ alkyl group, a C₁₋₆ alkyloxy group, a C₃₋₈ cycloalkyloxy group, a C₂₋₆ alkenyloxy group, a C₆₋₁₀ aryloxy group, a (C₆₋₁₀ aryl) C₁₋₆ alkyloxy group or a di (C₁₋₆ alkyl)amino group,

R^{4b} represents a C₁₋₆ alkyl group or a C₆₋₁₀ aryl group, and

R^{5b} and R^{6b} may be the same or different, and represent a C₁₋₆ alkyl group, a C₁₋₆ alkyloxy group, a C₆₋₁₀ aryloxy group, or a C₁₋₆ alkylthio group,

wherein any group represented by any group each represented by R^{3b}, R^{4b}, R^{5b} and R^{6b} may be substituted with halogen, and the C₃₋₈ cycloalkyl group, the C₆₋₁₀ aryl group, the aryl moiety of the (C₆₋₁₀ aryl) C₁₋₆ alkyl group, the C₃₋₈ cycloalkyloxy group, the C₆₋₁₀ aryloxy group, and the aryl moiety of the (C₆₋₁₀ aryl) C₁₋₆ alkyloxy group may be substituted with a C₁₋₆ alkyl group].

[14] The agent for controlling arthropod pests according to any one of [1] to [13], wherein, in the formula (I), G is hydrogen, a group represented by:

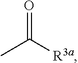

or a group represented by:

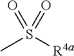

[wherein R^{3a} represents a C₁₋₆ alkyl group, a C₃₋₈ cycloalkyl group, a C₂₋₆ alkenyloxy group, a C₆₋₁₀ aryl group, a C₁₋₆ alkyloxy group or a di (C₁₋₆ alkyl)amino group, and R^{4a} represents a C₁₋₆ alkyl group,

wherein any group represented by R^{3a} and R^{4a} may be substituted with halogen, and the C₃₋₈ cycloalkyl group and the C₆₋₁₀ aryl group may be substituted with a C₁₋₆ alkyl group].

[15] A method for controlling arthropod pests comprising a step of treating an arthropod pest or a place where an arthropod pest lives with the effective amount of a pyridazinone compound shown by the formula (I):

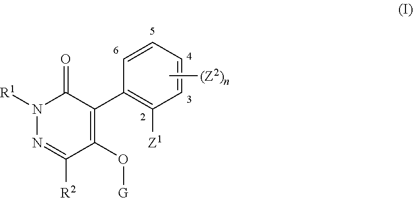

[wherein R¹ represents a C₁₋₆ alkyl group or a (C₁₋₆ alkyloxy) C₁₋₆ alkyl group,

R² represents hydrogen or a C₁₋₆ alkyl group, G represents hydrogen, a group represented by:

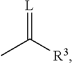

a group represented by:

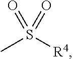

or a group represented by:

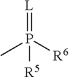

{wherein L represents oxygen or sulfur,

R³ represents a C₁₋₆ alkyl group, a C₃₋₈ cycloalkyl group, a C₂₋₆ alkenyl group, a C₂₋₆ alkynyl group, a C₆₋₁₀ aryl group, a (C₆₋₁₀ aryl) C₁₋₆ alkyl group, a C₁₋₆ alkyloxy group, a C₃₋₈ cycloalkyloxy group, a C₂₋₆ alkenyloxy group, a C₃₋₆ alkynyloxy group, a C₆₋₁₀ aryloxy group, a (C₆₋₂₀ aryl)C₁₋₆ alkyloxy group, a di (C₁₋₆ alkyl)amino group, a di (C₂₋₆ alkenyl)amino group, a (C₁₋₆ alkyl) (C₆₋₁₀ aryl)amino group or a 3- to 8-membered nitrogen-containing heterocyclic group,

R⁴ represents a C₂₋₆ alkyl group, a C₆₋₂₀ aryl group or a di (C₁₋₆ alkyl)amino group, and

R⁵ and R⁶ may be the same or different, and represent a C₂₋₆ alkyl group, a C₃₋₈ cycloalkyl group, a C₂₋₆ alkenyl group, a C₆₋₂₀ aryl group, a C₂₋₆ alkyloxy group, a C₃₋₈ cycloalkyloxy group, a C₆₋₂₀ aryloxy group, a (C₆₋₁₀ aryl)C₁₋₆ alkyloxy group, a C₂₋₆ alkylthio group or a di (C₁₋₆ alkyl)amino group,

wherein any group each represented by R³, R⁴, R⁵ and R⁶ may be substituted with halogen, and any of the C₃₋₈ cycloalkyl group, the C₆₋₂₀ aryl group, the aryl moiety of the (C₆₋₁₀ aryl) C₁₋₆ alkyl group, the C₃₋₈ cycloalkyloxy group, the C₆₋₂₀ aryloxy group, the aryl moiety of the (C₆₋₁₀ aryl)C₁₋₆ alkyloxy group, the aryl moiety of the (C₁₋₆ alkyl) (C₆₋₁₀ aryl)amino group and the 3- to 8-membered nitrogen-containing heterocyclic group may be substituted with a C₂₋₆ alkyl group},

Z⁴ represents a C₂₋₆ alkyl group,

Z² represents a C₂₋₆ alkyl group, a C₃₋₈ cycloalkyl group, a C₂₋₆ alkynyl group, a C₂₋₆ haloalkyl group, a C₆₋₂₀ aryl group, a 5- or 6-membered heteroaryl group, a C₂₋₆ alkyloxy group, a C₁₋₆ haloalkyloxy group, halogen, a cyano group or a nitro group, wherein the C₃₋₈ cycloalkyl group, the C₆₋₂₀ aryl group and the 5- or 6-membered heteroaryl group represented by Z² may be substituted with at least one group selected from the group consisting of halogens and C₂₋₆ alkyl groups, and

n represents 0, 1, 2, 3, or 4, and when n is 2, 3, or 4, each of Z² may be the same or different].

[15-2] The method for controlling arthropod pests according to [15-1], wherein, in the formula (I), n is 1, 2, or 3.

[15-3] The method for controlling arthropod pests according to [15-1], wherein, in the formula (I), n is 0, and Z¹ is a C₂₋₆ alkyl group.

[15-4] The method for controlling arthropod pests according to [15-1] or [15-2], wherein, in the formula (I), n is 1 or 2, and the binding position of Z² is a 4- and/or 6-positions on the benzene ring.

[15-5] The method for controlling arthropod pests according to any one of [15-1], [15-2], and [15-4], wherein, in the formula (I), Z¹ is a C₂₋₃ alkyl group, and Z² is a C₂₋₃ alkyl group, a C₃₋₆ cycloalkyl group, a C₂₋₃ alkynyl group, a C₂₋₃ alkyloxy group, halogen, a cyano group or a nitro group, or a phenyl group which may be substituted with at least one group selected from the group consisting of halogens and C₂₋₃ alkyl groups.

[15-6] The method for controlling arthropod pests according to any one of [15-1], [15-2], [15-4], and [15-5], wherein, in the formula (I), Z¹ is a C₂₋₃ alkyl group, and Z² is a C₂₋₃ alkyl group.

[15-7] The method for controlling arthropod pests according to any one of [15-1] and [15-2], wherein, in the formula (I), n is 1, and the binding position of Z² is a 5-position on the benzene ring.

[15-8] The method for controlling arthropod pests according to any one of [15-1], [15-2], and [15-7], wherein, in the formula (I), n is 1, the binding position of Z² is a 5-position on the benzene ring, Z¹ is a C₂₋₃ alkyl group, and Z² is a phenyl group which may be substituted with at least one group selected from the group consisting of halogens and C₂₋₃ alkyl groups.

[15-9] The method for controlling arthropod pests according to any one of [15-1] to [15-8], wherein, in the formula (I), R¹ is a C₁₋₃ alkyl group or a (C₁₋₃ alkyloxy) C₁₋₃ alkyl group.

[15-10] The method for controlling arthropod pests according to any one of [15-1] to [15-9], wherein, in the formula (I), R¹ is a methyl group.

[15-11] The method for controlling arthropod pests according to any one of [15-1] to [15-10], wherein, in the formula (I), R² is hydrogen or a C₁₋₃ alkyl group.

[15-12] The method for controlling arthropod pests according to any one of [15-1] to [15-11], wherein, in the formula (I), R² is hydrogen or a methyl group.

[15-13] The method for controlling arthropod pests according to any one of [15-1] to [15-12], wherein, in the formula (I), G is hydrogen, a group represented by:

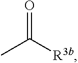

a group represented by:

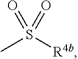

or a group represented by:

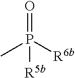

[wherein R^{3b} represents a C₁₋₆ alkyl group, a C₃₋₈ cycloalkyl group, a C₂₋₆ alkenyl group, a C₂₋₆ alkynyl group, a C₆₋₁₀ aryl group, a (C₆₋₁₀ aryl) C₁₋₆ alkyl group, a C₁₋₆ alkyloxy group, a C₃₋₈ cycloalkyloxy group, a C₂₋₆ alkenyloxy group, a C₆₋₁₀ aryloxy group, a (C₆₋₁₀ aryl) C₁₋₆ alkyloxy group or a di (C₁₋₆ alkyl)amino group,

R^{4b} represents a C₁₋₆ alkyl group or a C₆₋₁₀ aryl group, and

R^{5b} and R^{6b} may be the same or different, and represent a C₁₋₆ alkyl group, a C₁₋₆ alkyloxy group, a C₆₋₁₀ aryloxy group, or a C₁₋₆ alkylthio group,

wherein any group represented by any group each represented by R^{3b}, R^{4b}, R^{5b} and R^{6b} may be substituted with halogen, and any of the C₃₋₈ cycloalkyl group, the C₆₋₁₀ aryl group, the aryl moiety of the (C₆₋₁₀ aryl) C₁₋₆ alkyl group, the C₃₋₈ cycloalkyloxy group, the C₆₋₁₀ aryloxy group, and the aryl moiety of the (C₆₋₁₀ aryl) C₁₋₆ alkyloxy group may be substituted with a C₁₋₆ alkyl group].

[15-14] The method for controlling arthropod pests according to any one of [15-1] to [15-13], wherein, in the formula (I), G is hydrogen, a group represented by:

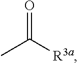

or a group represented by:

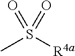

[wherein R^{3a} represents a C₁₋₆ alkyl group, a C₃₋₈ cycloalkyl group, a C₂₋₆ alkenyloxy group, a C₆₋₁₀ aryl group, a C₁₋₆ alkyloxy group or a di (C₁₋₆ alkyl)amino group, and

R^{4a} represents a C₁₋₆ alkyl group,

wherein any group represented by R^{3a} and R^{4a} may be substituted with halogen, and the C₃₋₈ cycloalkyl group and the C₆₋₁₀ aryl group may be substituted with a C₁₋₆ alkyl group].

[16] A use of the pyridazinone compound as described in [1] for controlling arthropod pests.

The substituents represented by R², R², R³, R⁴, R⁵, R⁶, Z¹, and Z² of the present compounds will be described.

The C₁₋₆ alkyl group means an alkyl group of 1 to 6 carbon atoms, and examples include a methyl group, an ethyl group, a propyl group, an isopropyl group, a butyl group, an isobutyl group, a 1-methylbutyl group, a tert-butyl group, a pentyl group, a sec-pentyl group, an isopentyl group, a neopentyl group, a hexyl group, and an isohexyl group.

The C₃₋₈ cycloalkyl group means a cycloalkyl group of 3 to 8 carbon atoms, and examples include a cyclopropyl group, a cyclopentyl group, and a cyclohexyl group.

The C₂₋₆ alkenyl group means an alkenyl group of 2 to 6 carbon atoms, and examples include a vinyl group, an allyl group, a 1-buten-3-yl group, and a 3-buten-1-yl group.

The C₂₋₆ alkynyl group means an alkynyl group of 2 to 6 carbon atoms, and examples include a propargyl group and a 2-butynyl group.

The C₆₋₁₀ aryl group means an aryl group of 6 to 10 carbon atoms, and examples include a phenyl group and a naphthyl group.

The (C₆₋₁₀ aryl) C₁₋₆ alkyl group means a C₁₋₆ alkyl group substituted with a C₆₋₁₀ aryl group, and examples include a benzyl group and a phenethyl group.

The C₁₋₆ alkyloxy group means an alkyloxy group of 1 to 6 carbon atoms, and examples include a methoxy group, an ethoxy group, a propoxy group, and an isopropoxy group.

The C₃₋₈ cycloalkyloxy group means a cycloalkyloxy group of 3 to 8 carbon atoms, and examples include a cyclopropyloxy group and a cyclopentyloxy group.

The C₂₋₆ alkenyloxy group means an alkenyloxy group of 2 to 6 carbon atoms, and examples include a vinyloxy group and an allyloxy group.

The C₃₋₆ alkynyloxy group means an alkynyloxy group of 3 to 6 carbon atoms, and examples include a propargyloxy group and a 2-butynyloxy group.

The C₆₋₁₀ aryloxy group means an aryloxy group of 6 to 10 carbon atoms, and examples include a phenoxy group and a naphthyloxy group.

The (C₆₋₁₀ aryl)C₁₋₆ alkyloxy group means a C₁₋₆ alkyloxy group substituted with a C₆₋₁₀ aryl group, and examples include a benzyloxy group and a phenethyloxy group.

The di (C₁₋₆ alkyl)amino group means an amino group substituted with two same or different C₁₋₆ alkyl groups, and examples include a dimethylamino group, a diethylamino group, and an N-ethyl-N-methylamino group.

The di (C₂₋₆ alkenyl)amino group means an amino group substituted with two same or different C₂₋₆ alkenyl groups, and examples include a diallylamino group and a di (3-butenyl)amino group.

The (C₁₋₆ alkyl) (C₆₋₁₀ aryl)amino group means an amino group substituted with a C₂₋₆ alkyl group and a C₆₋₂₀ aryl group, and examples include a methylphenylamino group and an ethylphenylamino group.

The C₂₋₆ alkylthio group means an alkylthio group of 1 to 6 carbon atoms, and examples include a methylthio group, an ethylthio group, a propylthio group, and an isopropylthio group.

The (C₁₋₆ alkyloxy) C₁₋₆ alkyl group means a C₂₋₆ alkyl group substituted with a C₂₋₆ alkyloxy group, and examples include a methoxyethyl group and an ethoxyethyl group.

The 3- to 8-membered nitrogen-containing heterocyclic group means an aromatic or alicyclic 3- to 8-membered heterocyclic group containing 1 to 3 nitrogens and optionally further containing 1 to 3 oxygens and/or sulfurs, and examples include a 1-pyrazolyl group, a 2-pyridyl group, a 2-pyrimidinyl group, a 2-thiazolyl group, a pyrrolidino group, a piperidino group, and a morpholino group.

The 5- or 6-membered heteroaryl group means an aromatic 5- or 6-membered heterocyclic group containing 1 to 3 hetero atoms selected from nitrogen, oxygen, and sulfur, and examples include a 2-pyridyl group, a 3-pyridyl group, a 4-pyridyl group, a 2-pyrimidinyl group, a 2-thienyl group, a 3-thienyl group, a 2-furyl group, a 3-furyl group, a 2-thiazolyl group, a 4-thiazolyl group, a 5-thiazolyl group, a 2-oxazolyl group, a 4-oxazolyl group, a 5-oxazolyl group, and a 1-pyrazolyl group.

Examples of the halogen in the present compound include fluorine, chlorine, bromine, iodine, and the like.

The C₁₋₆ haloalkyl group represented by Z² means a C₂₋₆ alkyl group substituted with fluorine, chlorine, bromine, iodine, or the like, and examples include a trifluoromethyl group and a 2,2,2-trichloroethyl group.

The C₁₋₆ haloalkyloxy group represented by Z² means a C₁₋₆ alkyloxy group substituted with fluorine, chlorine, bromine, or iodine, and examples include a trifluoromethoxy group and a 2,2,2-trifluoroethoxy group.

Any group each represented by R³, R⁴, R⁵, and R⁶ may be substituted with halogen, and the C₃₋₈ cycloalkyl group, the C₆₋₁₀ aryl group, the aryl moiety of the (C₆₋₁₀ aryl)C₁₋₆ alkyl group, the C₃₋₈ cycloalkyloxy group, the C₆₋₁₀ aryloxy group, the aryl moiety of the (C₆₋₁₀ aryl)C₁₋₆ alkyloxy group, the aryl moiety of the (C₁₋₆ alkyl) (C₆₋₁₀ aryl)amino group and the 3- to 8-membered nitrogen-containing heterocyclic group may be substituted with a C₁₋₆ alkyl group.

Among the groups represented by Z², any of the C₃₋₈ cycloalkyl group, the C₆₋₁₀ aryl group and the 5- or 6-membered heteroaryl group may be substituted with at least one group selected from the group consisting of halogens and C₁₋₆ alkyl groups described above.

The compound of the formula (I) includes those listed below.

The pyridazinone compound, wherein, in the formula (I), n is 1, 2, or 3.

The pyridazinone compound, wherein, in the formula (I), n is 0, and Z¹ is a C₂₋₆ alkyl group.

The pyridazinone compound, wherein, in the formula (I), n is 1 or 2, and when n is 2, two Z² may be the same or different, and the binding position of Z² is a 4- and/or 6-positions on the benzene ring.

The pyridazinone compound, wherein, in the formula (I), G is hydrogen, a group represented by:

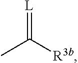

a group represented by:

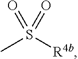

or a group represented by:

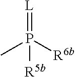

{wherein R^{3b} represents a C₁₋₆ alkyl group, a C₃₋₈ cycloalkyl group, a C₂₋₆ alkenyl group, a C₂₋₆ alkynyl group, a C₆₋₁₀ aryl group, a (C₆₋₁₀ aryl) C₁₋₆ alkyl group, a C₁₋₆ alkyloxy group, a C₃₋₈ cycloalkyloxy group, a C₂₋₆ alkenyloxy group, a C₆₋₁₀ aryloxy group, a (C₆₋₁₀ aryl) C₁₋₆ alkyloxy group, or a di (C₁₋₆ alkyl)amino group,

R^{4b} represents a C₁₋₆ alkyl group or a C₆₋₁₀ aryl group, and

R^{5b} and R^{6b} may be the same or different, and represent a C₁₋₆ alkyl group, a C₁₋₆ alkyloxy group, a C₆₋₁₀ aryloxy group, or a C₁₋₆ alkylthio group,

wherein any group represented by any group each represented by R^{3b}, R^{4b}, R^{5b}, and R^{6b} may be substituted with halogen, and any of the C₃₋₈ cycloalkyl group, the C₆₋₁₀ aryl group, the aryl moiety of the (C₆₋₁₀ aryl) C₁₋₆ alkyl group, the C₃₋₈ cycloalkyloxy group, the C₆₋₁₀ aryloxy group, and the aryl moiety of the (C₆₋₁₀ aryl) C₁₋₆ alkyloxy group may be substituted with a C₁₋₆ alkyl group}.

The pyridazinone compound, wherein, in the formula (I), G is hydrogen, a group represented by:

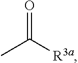

or a group represented by:

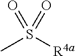

{wherein R^{3a} represents a C₁₋₆ alkyl group, a C₃₋₈ cycloalkyl group, a C₆₋₁₀ aryl group, a C₁₋₆ alkyloxy group, a C₂₋₆ alkenyloxy group, or a di (C₁₋₆ al+kyl)amino group, and

R^{4a} represents a C₁₋₆ alkyl group,

wherein any group represented by R^{3a} and R^{4a} may be substituted with halogen, and the C₃₋₈ cycloalkyl group and the C₆₋₁₀ aryl group may be both substituted with a C₁₋₆ alkyl group}.

The pyridazinone compound, wherein, in the formula (I), R¹ is a C₁₋₃ alkyl group or a (C₁₋₃ alkyloxy)C₁₋₃ alkyl group.

The pyridazinone compound, wherein, in the formula (I), R² is hydrogen or a C₁₋₃ alkyl group.

The pyridazinone compound, wherein, in the formula (I), R² is hydrogen or a methyl group.

The pyridazinone compound, wherein, in the formula (I), Z¹ is a C₁₋₃ alkyl group, and Z² is a C₁₋₃ alkyl group.

The pyridazinone compound, wherein, in the formula (I), Z¹ is a C₁₋₃ alkyl group, and Z² is a C₁₋₃ alkyl group, a C₃₋₆ cycloalkyl group, a C₂₋₃ alkynyl group, a C₁₋₃ alkyloxy group, a C₁₋₃ haloalkyl group, a C₁₋₃ haloalkyloxy group, halogen, a cyano group or a nitro group, or a phenyl group which may be substituted with at least one group selected from the group consisting of halogens and C₁₋₃ alkyl groups or the 5- or 6-membered heteroaryl group.

The pyridazinone compound, wherein, in the formula (I), R¹ is a C₁₋₃ alkyl group or a (C₁₋₃ alkyloxy) C₁₋₃ alkyl group, and R² is hydrogen or a C₁₋₃ alkyl group.

The pyridazinone compound, wherein, in the formula (I), R¹ is a C₁₋₃ alkyl group or a (C₁₋₃ alkyloxy) C₁₋₃ alkyl group, and R² is hydrogen or a methyl group.

The pyridazinone compound, wherein, in the formula (I), R² is hydrogen or a C₁₋₃ alkyl group, and G is hydrogen, a group represented by:

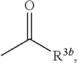

a group represented by:

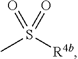

or a group represented by:

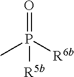

{wherein R^{3b}, R^{4b}, R^{5b}, and R^{6b} have the same meaning as described above}.

The pyridazinone compound, wherein, in the formula (I), R² is hydrogen or a C₁₋₃ alkyl group, and G is hydrogen, a group represented by:

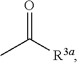

or a group represented by:

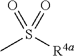

{wherein R^{3a} and R^{4a} have the same meaning as described above}.

The pyridazinone compound, wherein, in the formula (I), R² is hydrogen or a methyl group, and G is hydrogen, a group represented by:

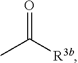

a group represented by:

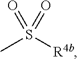

or a group represented by:

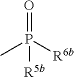

{wherein R^{3b}, R^{4b}, R^{5b}, and R^{6b} have the same meaning as described above}.

The pyridazinone compound, wherein, in the formula (I), R² is hydrogen or a methyl group, and G is hydrogen, a group represented by:

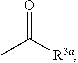

or a group represented by:

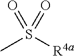

{wherein R^{3a} and R^{4a} have the same meaning as described above}.

The pyridazinone compound, wherein, in the formula (I), R⁴ is a C₁₋₃ alkyl group or a (C₁₋₃ alkyloxy)C₁₋₃ alkyl group, R² is hydrogen or a C₁₋₃ alkyl group, and G is hydrogen, a group represented by:

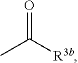

a group represented by:

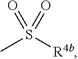

or a group represented by:

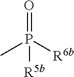

{wherein R^{3b}, R^{4b}, R^{5b}, and R^{6b} have the same meaning as described above}.

The pyridazinone compound, wherein, in the formula (I), R¹ is a C₁₋₃ alkyl group or a (C₁₋₃ alkyloxy)C₁₋₃ alkyl group, R² is hydrogen or a C₁₋₃ alkyl group, and G is hydrogen, a group represented by:

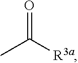

or a group represented by:

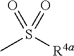

{wherein R^{3a} and R^{4a} have the same meaning as described above}.

The pyridazinone compound, wherein, in the formula (I), R⁴ is a C₁₋₃ alkyl group or a (C₁₋₃ alkyloxy)C₁₋₃ alkyl group, R² is hydrogen or a methyl group, and G is hydrogen, a group represented by:

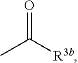

a group represented by:

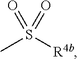

or a group represented by:

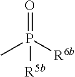

{wherein R^{3b}, R^{4b}, R^{5b}, and R^{6b} have the same meaning as described above}.

The pyridazinone compound, wherein, in the formula (I), R⁴ is a C₁₋₃ alkyl group or a (C₁₋₃ alkyloxy)C₁₋₃ alkyl group, R² is hydrogen or a methyl group, and G is hydrogen, a group represented by:

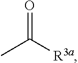

or a group represented by:

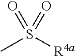

{wherein R^{3a} and R^{4a} have the same meaning as described above}. The pyridazinone compound, wherein, in the formula (I), R⁴ is a C₁₋₃ alkyl group or a (C₁₋₃ alkyloxy)C₁₋₃ alkyl group, R² is hydrogen or a C₁₋₃ alkyl group,

n is 1 or 2, and when n is 2, two Z² may be the same or different, the binding position of Z² is a 4- and/or 6-positions on the benzene ring,

Z¹ is a C₁₋₆ alkyl group, and

Z² is a C₁₋₆ alkyl group.

The pyridazinone compound, wherein, in the formula (I), R¹ is a C₁₋₃ alkyl group or a (C₁₋₃ alkyloxy) C₁₋₃ alkyl group, R² is hydrogen or a C₁₋₃ alkyl group,

n is 1 or 2, and when n is 2, two Z² may be the same or different, the binding position of Z² is a 4- and/or 6-positions on the benzene ring,

Z¹ is a C₁₋₆ alkyl group, and

Z² is a C₁₋₆ alkyl group, a C₃₋₈ cycloalkyl group, a C₂₋₆ alkynyl group, a C₁₋₆ alkyloxy group, a C₁₋₆ haloalkyl group, a C₁₋₆ haloalkyloxy group, halogen, a cyano group or a nitro group, or a C₆₋₁₀ aryl group which may be substituted with at least one group selected from the group consisting of halogens and C₁₋₆ alkyl groups or the 5- or 6-membered heteroaryl group.

The pyridazinone compound, wherein, in the formula (I), R¹ is a C₁₋₃ alkyl group or a (C₁₋₃ alkyloxy) C₁₋₃ alkyl group, R² is hydrogen or a C₁₋₃ alkyl group, and G is hydrogen, a group represented by:

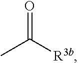

a group represented by:

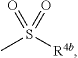

or a group represented by:

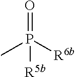

{wherein R^{3b}, R^{4b}, R^{5b}, and R^{6b} have the same meaning as described above},

n is 1 or 2, and when n is 2, two Z² may be the same or different, the binding position of Z² is a 4- and/or 6-positions on the benzene ring,

Z¹ is a C₁₋₆ alkyl group, and

Z² is a C₁₋₆ alkyl group.

The pyridazinone compound, wherein, in the formula (I), R¹ is a C₁₋₃ alkyl group or a (C₁₋₃ alkyloxy)C₁₋₃ alkyl group, R² is hydrogen or a C₁₋₃ alkyl group, and G is hydrogen, a group represented by:

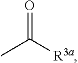

or a group represented by:

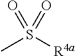

{wherein R^{3a} and R^{4a} have the same meaning as described above},

n is 1 or 2, and when n is 2, two Z² may be the same or different,

the binding position of Z² is a 4- and/or 6-positions on the benzene ring,

Z¹ is a C₁₋₆ alkyl group, and

Z² is a C₁₋₆ alkyl group.

The pyridazinone compound, wherein, in the formula (I), R¹ is a C₁₋₃ alkyl group or a (C₁₋₃ alkyloxy)C₁₋₃ alkyl group, R² is hydrogen or a C₁₋₃ alkyl group, and G is hydrogen, a group represented by:

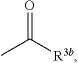

a group represented by:

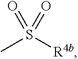

or a group represented by:

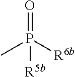

{wherein R^{3b}, R^{4b}, R^{5b}, and R^{6b} have the same meaning as described above},

n is 1 or 2, and when n is 2, two Z² may be the same or different,

the binding position of Z² is a 4- and/or 6-positions on the benzene ring,

Z¹ is a C₁₋₆ alkyl group, and

Z² is a C₁₋₆ alkyl group, a C₃₋₈ cycloalkyl group, a C₂₋₆ alkynyl group, a C₁₋₆ alkyloxy group, a C₁₋₆ haloalkyl group, a C₁₋₆ haloalkyloxy group, halogen, a cyano group or a nitro group, or a C₆₋₁₀ aryl group which may be substituted with at least one group selected from the group consisting of halogens and C₁₋₆ alkyl groups or the 5- or 6-membered heteroaryl group.

The pyridazinone compound, wherein, in the formula (I), R¹ is a C₁₋₃ alkyl group or a (C₁₋₃ alkyloxy) C₁₋₃ alkyl group, R² is hydrogen or a C₁₋₃ alkyl group, and G is hydrogen, a group represented by:

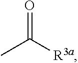

or a group represented by:

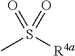

{wherein R^{3a} and R^{4a} have the same meaning as described above},

n is 1 or 2, and when n is 2, two Z² may be the same or different, the binding position of Z² is a 4- and/or 6-positions on the benzene ring,

Z¹ is a C₁₋₆ alkyl group, and

Z² is a C₁₋₆ alkyl group, a C₃₋₈ cycloalkyl group, a C₂₋₆ alkynyl group, a C₁₋₆ alkyloxy group, a C₁₋₆ haloalkyl group, a C₁₋₆ haloalkyloxy group, halogen, a cyano group or a nitro group, or a C₆₋₁₀ aryl group which may be substituted with at least one group selected from the group consisting of halogens and C₁₋₆ alkyl groups.

The pyridazinone compound, wherein, in the formula (I), R¹ is a C₁₋₃ alkyl group or a (C₁₋₃ alkyloxy)C₁₋₃ alkyl group, R² is hydrogen or a methyl group,

n is 1 or 2, and when n is 2, two Z² may be the same or different,

the binding position of Z² is a 4- and/or 6-positions on the benzene ring,

Z¹ is a C₁₋₆ alkyl group, and

Z² is a C₁₋₆ alkyl group.

The pyridazinone compound, wherein, in the formula (I), R¹ is a C₁₋₃ alkyl group or a (C₁₋₃ alkyloxy)C₁₋₃ alkyl group, R² is hydrogen or a methyl group,

n is 1 or 2, and when n is 2, two Z² may be the same or different,

the binding position of Z² is a 4- and/or 6-positions on the benzene ring,

Z¹ is a C₁₋₆ alkyl group, and

Z² is a C₁₋₆ alkyl group, a C₃₋₈ cycloalkyl group, a C₂₋₆ alkynyl group, a C₁₋₆ alkyloxy group, a C₁₋₆ haloalkyl group, a C₁₋₆ haloalkyloxy group, halogen, a cyano group or a nitro group, or a C₆₋₁₀ aryl group which may be substituted with at least one group selected from the group consisting of halogens and C₁₋₆ alkyl groups.

The pyridazinone compound, wherein, in the formula (I), R¹ is a C₁₋₃ alkyl group or a (C₁₋₃ alkyloxy)C₁₋₃ alkyl group, R² is hydrogen or a methyl group, and G is hydrogen, a group represented by:

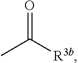

a group represented by:

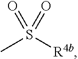

or a group represented by:

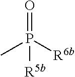

{wherein R^{3b}, R^{4b}, R^{5b}, and R^{6b} have the same meaning as described above},

n is 1 or 2, and when n is 2, two Z² may be the same or different,

the binding position of Z² is a 4- and/or 6-positions on the benzene ring,

Z¹ is a C₁₋₆ alkyl group, and

Z² is a C₁₋₆ alkyl group.

The pyridazinone compound, wherein, in the formula (I), R¹ is a C₁₋₃ alkyl group or a (C₁₋₃ alkyloxy)C₁₋₃ alkyl group, R² is hydrogen or a methyl group, and G is hydrogen, a group represented by:

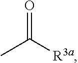

or a group represented by:

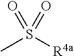

{wherein R^{3a} and R^{4a} have the same meaning as described above},

n is 1 or 2, and when n is 2, two Z² may be the same or different, the binding position of Z² is a 4- and/or 6-positions on the benzene ring,

Z¹ is a C₁₋₆ alkyl group, and

Z² is a C₁₋₆ alkyl group.

The pyridazinone compound, wherein, in the formula (I), R² is a C₁₋₃ alkyl group or a (C₁₋₃ alkyloxy)C₁₋₃ alkyl group, R² is hydrogen or a methyl group, and G is hydrogen, a group represented by:

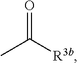

a group represented by:

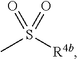

or a group represented by:

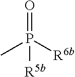

{wherein R^{3b}, R^{4b}, R^{5b}, and R^{6b} have the same meaning as described above},

n is 1 or 2, and when n is 2, two Z² may be the same or different,

the binding position of Z² is a 4- and/or 6-positions on the benzene ring,

Z¹ is a C₁₋₆ alkyl group, and

Z² is a C₁₋₆ alkyl group, a C₃₋₈ cycloalkyl group, a C₂₋₆ alkynyl group, a C₁₋₆ alkyloxy group, a C₁₋₆ haloalkyl group, a C₁₋₆ haloalkyloxy group, halogen, a cyano group or a nitro group, or a C₆₋₁₀ aryl group which may be substituted with at least one group selected from the group consisting of halogens and C₁₋₆ alkyl groups.

The pyridazinone compound, wherein, in the formula (I), R¹ is a C₁₋₃ alkyl group or a (C₁₋₃ alkyloxy) C₁₋₃ alkyl group, R² is hydrogen or a methyl group, and G is hydrogen, a group represented by:

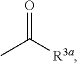

or a group represented by:

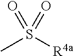

{wherein R^{3a} and R^{4a} have the same meaning as described above},

n is 1 or 2, and when n is 2, two Z² may be the same or different,

the binding position of Z² is a 4- and/or 6-positions on the benzene ring,

Z¹ is a C₁₋₆ alkyl group, and

Z² is a C₁₋₆ alkyl group, a C₃₋₈ cycloalkyl group, a C₂₋₆ alkynyl group, a C₁₋₆ alkyloxy group, a C₁₋₆ haloalkyl group, a C₁₋₆ haloalkyloxy group, halogen, a cyano group or a nitro group, or a C₆₋₁₀ aryl group which may be substituted with at least one group selected from the group consisting of halogens and C₁₋₆ alkyl groups.

The pyridazinone compound, wherein, in the formula (I), n in the formula (I) is 1, and the binding position of Z² is a 5-position on the benzene ring.

The pyridazinone compound, wherein, in the formula (I), R¹ is a C₁₋₃ alkyl group or a (C₁₋₃ alkyloxy) C₁₋₃ alkyl group, R² is hydrogen or a C₁₋₃ alkyl group,

n is 1, the binding position of Z² is a 5-position on the benzene ring,

Z¹ is a C₁₋₆ alkyl group, and

Z² is a C₆₋₁₀ aryl group which may be substituted with at least one group selected from the group consisting of halogens and C₁₋₆ alkyl groups.

The pyridazinone compound, wherein, in the formula (I), R¹ is a C₁₋₃ alkyl group or a (C₁₋₃ alkyloxy) C₁₋₃ alkyl group, R² is hydrogen or a C₁₋₃ alkyl group, and G is hydrogen, a group represented by:

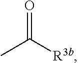

a group represented by:

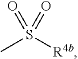

or a group represented by:

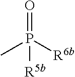

{wherein R^{3b}, R^{4b}, R^{5b}, and R^{6b} have the same meaning as described above},

n is 1, the binding position of Z² is a 5-position on the benzene ring,

Z¹ is a C₁₋₆ alkyl group, and

Z² is a C₆₋₁₀ aryl group which may be substituted with at least one group selected from the group consisting of halogens and C₁₋₆ alkyl groups.

The pyridazinone compound, wherein, in the formula (I), R¹ is a C₁₋₃ alkyl group or a (C₁₋₃ alkyloxy) C₁₋₃ alkyl group, R² is hydrogen or a C₁₋₃ alkyl group, and G is hydrogen, a group represented by:

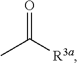

or a group represented by:

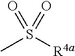

{wherein R^{ia} and R^{4a} have the same meaning as described above},

n is 1, the binding position of Z² is a 5-position on the benzene ring,

Z¹ is a C₁₋₆ alkyl group, and

Z² is a C₆₋₁₀ aryl group which may be substituted with at least one group selected from the group consisting of halogens and C₁₋₆ alkyl groups (further preferably C₁₋₃ alkyl groups).

The pyridazinone compound, wherein, in the formula (I), R¹ is a C₁₋₃ alkyl group or a (C₁₋₃ alkyloxy) C₁₋₃ alkyl group, R² is hydrogen or a methyl group,

n is 1, the binding position of Z² is a 5-position on the benzene ring,

Z¹ is a C₁₋₆ alkyl group, and

Z² is a C₆₋₁₀ aryl group which may be substituted with at least one group selected from the group consisting of halogens and C₁₋₆ alkyl groups.

The pyridazinone compound, wherein, in the formula (I), R² is a C₁₋₃ alkyl group or a (C₁₋₃ alkyloxy) C₁₋₃ alkyl group, R² is hydrogen or a methyl group, and G is hydrogen, a group represented by:

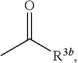

a group represented by:

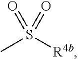

or a group represented by:

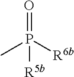

{wherein R^{3b}, R^{4b}, R^{5b}, and R^{6b} have the same meaning as described above},

n is 1, the binding position of Z² is a 5-position on the benzene ring,

Z² is a C₁₋₆ alkyl group, and

Z² is a C₆₋₁₀ aryl group which may be substituted with at least one group selected from the group consisting of halogens and C₁₋₆ alkyl groups.

The pyridazinone compound, wherein, in the formula (I), R² is a C₁₋₃ alkyl group or a (C₁₋₃ alkyloxy) C₁₋₃ alkyl group, R² is hydrogen or a methyl group, and G is hydrogen, a group represented by:

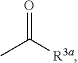

or a group represented by:

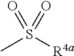

{wherein R^{3a} and R^{4a} have the same meaning as described above},

n is 1, the binding position of Z² is a 5-position on the benzene ring,

Z¹ is a C₁₋₆ alkyl group, and

Z² is a C₆₋₁₀ aryl group which may be substituted with at least one group selected from the group consisting of halogens and C₁₋₆ alkyl groups.

The pyridazinone compound in formula (I-1):

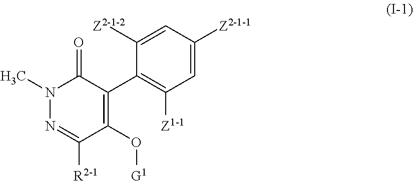

wherein R²⁻¹ is hydrogen or a C₁₋₃ alkyl group, G¹ is hydrogen, a C₁₋₃ alkylcarbonyl group which may be substituted with halogen, a C₁₋₄ alkoxycarbonyl group, a C₂₋₄ alkenyloxycarbonyl group, or a C₆₋₁₀ arylcarbonyl group,

Z¹⁻¹ is a C₁₋₃ alkyl group,

Z²⁻¹⁻¹ is a C₁₋₃ alkyl group, and

Z²⁻¹⁻² is hydrogen or a C₁₋₃ alkyl group.

The pyridazinone compound in the formula (I-1), wherein R²⁻¹ is hydrogen, a methyl group, or an ethyl group, G¹ is hydrogen, an acetyl group, a propionyl group, a methoxycarbonyl group, an ethoxycarbonyl group, a tert-butoxycarbonyl group, an allyloxycarbonyl group, or a benzoyl group,

Z¹⁻¹ is a methyl group or an ethyl group,

Z²⁻¹⁻¹ is a methyl group or an ethyl group, and

Z²⁻¹⁻² is hydrogen, a methyl group, or an ethyl group.

The pyridazinone compound in the formula (I-1), wherein R²⁻¹ is hydrogen or a C₁₋₃ alkyl group, G¹ is hydrogen, a C₁₋₃ alkylcarbonyl group which may be substituted with halogen, a C₁₋₄ alkoxycarbonyl group, a C₂₋₄ alkenyloxycarbonyl group, or a C₆₋₁₀ arylcarbonyl group,

Z¹⁻¹ is a C₁₋₃ alkyl group,

Z²⁻¹⁻¹ is a C₃₋₆ cycloalkyl group, a C₂₋₃ alkynyl group, a C₁₋₃ alkyloxy group, a C₁₋₃ haloalkyl group, a C₁₋₃ haloalkyloxy group, halogen, a cyano group or a nitro group, or a phenyl group which may be substituted with at least one group selected from the group consisting of halogens and C₁₋₃ alkyl groups or the 5- or 6-membered heteroaryl group, and

Z²⁻¹⁻² is hydrogen, a C₁₋₃ alkyl group, a C₃₋₆ cycloalkyl group, a C₂₋₃ alkynyl group, a C₁₋₃ alkyloxy group, or halogen.

The pyridazinone compound in the formula (I-1), wherein R²⁻¹ is hydrogen, a methyl group, or an ethyl group, G¹ is hydrogen, an acetyl group, a propionyl group, a methoxycarbonyl group, an ethoxycarbonyl group, a tert-butoxycarbonyl group, an allyloxycarbonyl group, or a benzoyl group,

Z¹⁻¹ is a methyl group or an ethyl group,

Z²⁻¹⁻¹ is a cyclopropyl group, an ethynyl group, a methoxy group, a trifluoromethyl group, a trifluoromethoxy group, chlorine, bromine, a phenyl group, a 4-methylphenyl group, a 4-chlorophenyl group, a 3-pyridyl group, a 4-pyridyl group, a 3-thienyl group, a 3-furyl group, a cyano group, or a nitro group, and

Z²⁻¹⁻² is hydrogen, a methyl group, an ethyl group, a cyclopropyl group, an ethynyl group, a methoxy group, chlorine, or bromine.

The pyridazinone compound in the formula (I-1), wherein R²⁻¹ is hydrogen or a C₁₋₃ alkyl group, G¹ is hydrogen, a C₁₋₃ alkylcarbonyl group which may be substituted with halogen, a C₁₋₄ alkoxycarbonyl group, a C₂₋₄ alkenyloxycarbonyl group, or a C₆₋₁₀ arylcarbonyl group,

Z¹⁻¹ is a C₁₋₃ alkyl group,

Z²⁻¹⁻¹ is hydrogen, a C₁₋₃ alkyl group, a C₃₋₆ cycloalkyl group, a C₂₋₃ alkynyl group, a C₁₋₃ alkyloxy group, or halogen, and

Z²⁻¹⁻² is a C₃₋₆ cycloalkyl group, a C₂₋₃ alkynyl group, a C₁₋₃ alkyloxy group, halogen, a cyano group, or a nitro group.

The pyridazinone compound in the formula (I-1), wherein R²⁻² is hydrogen, a methyl group, or an ethyl group, G¹ is hydrogen, an acetyl group, a propionyl group, a methoxycarbonyl group, an ethoxycarbonyl group, a tert-butoxycarbonyl group, an allyloxycarbonyl group, or a benzoyl group,

Z¹⁻¹ is a methyl group or an ethyl group,

Z²⁻¹⁻¹ is hydrogen, a methyl group, an ethyl group, a cyclopropyl group, an ethynyl group, a methoxy group, chlorine, or bromine, and

Z²⁻¹⁻² is a cyclopropyl group, an ethynyl group, a methoxy group, chlorine, bromine, a cyano group, or a nitro group.

The pyridazinone compound, wherein, in formula (I-2):

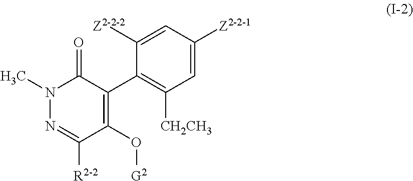

R²⁻² is hydrogen or a C₁₋₃ alkyl group, G² is hydrogen, a C₁₋₃ alkylcarbonyl group which may be substituted with halogen, a C₁₋₄ alkoxycarbonyl group, a C₂₋₄ alkenyloxycarbonyl group, or a C₆₋₁₀ arylcarbonyl group,

Z²⁻²⁻¹ is hydrogen or a C₁₋₃ alkyl group, and

Z²⁻²⁻² is hydrogen or a C₁₋₃ alkyl group.

The pyridazinone compound, wherein, in the formula (I-2), R²⁻² is hydrogen, a methyl group, or an ethyl group, G² is hydrogen, an acetyl group, a propionyl group, a methoxycarbonyl group, an ethoxycarbonyl group, a tert-butoxycarbonyl group, an allyloxycarbonyl group, or a benzoyl group,

Z²⁻²⁻¹ is hydrogen, a methyl group, or an ethyl group, and

Z²⁻²⁻² is hydrogen, a methyl group or an ethyl group.

The pyridazinone compound, wherein, in formula (I-3):

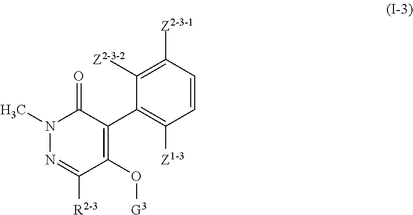

R²⁻³ is hydrogen or a C₁₋₃ alkyl group, G³ is hydrogen, a C₁₋₃ alkylcarbonyl group which may be substituted with halogen, a C₁₋₄ alkoxycarbonyl group, a C₂₋₄ alkenyloxycarbonyl group, or a C₆₋₁₀ arylcarbonyl group,

Z¹⁻³ is a C₁₋₃ alkyl group,

Z²⁻³⁻¹ is a phenyl group which may be substituted with at least one group selected from the group consisting of halogens and C₁₋₃ alkyl groups, and

Z²⁻³⁻¹ is hydrogen, a C₁₋₃ alkyl group, or halogen.

The pyridazinone compound, wherein, in the formula (I-3), R²⁻³ is hydrogen, a methyl group, or an ethyl group, G³ is hydrogen, an acetyl group, a propionyl group, a methoxycarbonyl group, an ethoxycarbonyl group, a tert-butoxycarbonyl group, an allyloxycarbonyl group, or a benzoyl group,

Z¹⁻³ is a methyl group or an ethyl group,

Z²⁻³⁻¹ is a phenyl group, a 2-fluorophenyl group, a 3-fluorophenyl group, a 4-fluorophenyl group, a 3,4-difluorophenyl group, a 2-chlorophenyl group, a 3-chlorophenyl group, a 4-chlorophenyl group, a 3-methyl phenyl group, or a 4-methyl phenyl group, and

Z²⁻³⁻² is hydrogen, a methyl group, or chlorine.

The control agent of the present invention contains the present composition and an inert carrier. The inert carrier includes a solid carrier, a liquid carrier, and a gas carrier. To the control agent of the present invention are generally further added auxiliaries for preparation such as a surfactant, an adhesive agent, a dispersant, and a stabilizer, and the mixture was formulated into a wettable powder, a wettable granule, a flowable, a granule, a dry flowable, an emulsifiable concentrate, an aqueous solvent, an oil solution, a smoking agent, an aerosol, a micro capsule, or the like. The control agent of the present invention contains generally 0.1 to 90% of the present composition in weight ratio.

Examples of the solid carrier include fine powders and granulated substances such as clays (e.g., kaolin, diatomaceous earth, synthetic hydrous silicon oxide, Fubasami clay, bentonite, and acid white clay), talc, other inorganic minerals (e.g., sericite, quarts powder, sulfur powder, activated carbon, calcium carbonate, and hydrated silica), and the like, and examples of the liquid carrier include water, alcohols (e.g., methanol and ethanol), ketones (e.g., acetone and methyl ethyl ketone), aromatic hydrocarbons (e.g., benzene, toluene, xylene, ethylbenzene, and methylnaphthalene), aliphatic hydrocarbons (e.g., n-hexane, cyclohexane, and kerosene), esters (e.g., ethyl acetate and butyl acetate), nitriles (e.g., acetonitrile and isobutylnitrile), ethers (e.g, dioxane and diisopropyl ether), acid amides (e.g., N,N-dimethylformamide and dimethylacetamide), halogenated hydrocarbons (e.g., dichloroethane, trichloroethylene, carbon tetrachloride), and the like.

Examples of the surfactant include alkyl sulfate esters, alkyl sulfonates, alkyl aryl sulfonates, alkyl aryl ethers and polyoxyethylene compounds, polyoxyethylene glycol ethers, polyhydric alcohol esters, and sugar alcohol derivatives thereof, and the like.

Examples of the other formulation auxiliaries include sticking agents and dispersing agents, specifically, casein, gelatin, polysaccharides (e.g., starch, gum arabic, cellulose derivatives, and alginic acid), lignin derivatives, bentonite, saccharides, synthetic water-soluble polymers (e.g., polyvinyl alcohol, polyvinyl pyrrolidone, and polyacrylic acids), PAP (acidic isopropyl phosphate), BHT (2,6-di-tert-butyl-4-methylphenol), BHA (a mixture of 2-tert-butyl-4-methoxyphenol and 3-tert-butyl-4-methoxyphenol), vegetable oils, mineral oils, fatty acids, fatty acid esters thereof, and the like.

The method for controlling arthropod pests of the present invention has a step of applying the effective amount of the present composition to the arthropod pests or a place where the arthropod pests live. Generally, the preparation containing the present compound is used for the method for controlling arthropod pests of the present invention.

When the method for controlling arthropod pests of the present invention is intended for the control of arthropod pests in agriculture and forestry field, the amount of the present compound to be applied is generally at a rate of 0.001 to 1000 g per 1000 m², and preferably at a rate of 0.01 to 100 g per 1000 m². Examples of the method for controlling arthropod pests of the present invention in this case include a method of directly applying a composition containing the present compound to an arthropod pest, a method of treating a plant where an arthropod pest lives with a composition containing the present compound, and a method of treating a soil where an arthropod pest lives with a composition containing the present compound.

When the method for controlling arthropod pests of the present invention is intended for the control of arthropod pests in epidemic prevention field, the amount of the present compound to be applied is at a rate of 0.001 to 10 mg/m³ in the case of applying to a space, and at a rate of 0.001 to 100 mg/m² in the case of applying to a plane. Examples of the method for controlling arthropod pests of the present invention in this case include a method of directly applying a composition containing the present compound to an arthropod pest, a method of applying a composition containing the present compound to a space where an arthropod pest lives, and a method of applying a composition containing the present compound to a surface where an arthropod pest lives.

The agent of the present invention can be used as mixed or in combination with another agent for arthropod pests, bactericide, synergist, attractant, repellent, safener, fertilizer, soil-improving agent, and the like.

Examples of the arthropod pest to which the control agent of the present invention has an effect include insects and mites. Examples of the arthropod pest specifically include those listed below.

### Hemiptera:

Delphacidae such as Laodelphax striatellus, Nilaparvata lugens, Sogatella furcifera and the like,

Deltocephalidae such as Nephotettix cincticeps, Nephotettix virescens and the like,

Aphididae such as Aphis gossypii, Myzus persicae, Brevicoryne brassicae, Macrosiphum euphorbiae, Aulacorthum solani, Rhopalosiphum padi, Toxoptera citricidus and the like, Pentatomidae such as Nezara antennata, Riptortus clavetus, Leptocorisa chinensis, Eysarcoris parvus, Halyomorpha mista, Lygus lineolaris and the like, Aleyrodidae such as Trialeurodes vaporariorum, Bemisia tabaci, Bemisia argentifolii and the like,

Coccidae such as Aonidiella aurantii, Comstockaspis perniciosa, Unaspis citri, Ceroplastes rubens, Icerya purchasi, Pseudococcus comstockli, Planococcus citri and the like,

Tingidae,

Psyllidae such as Pear psylla, and the like;

### Lepidoptera:

Pyralidae such as Chilo suppressalis, Tryporyza incertulas, Cnaphalocrocis medinalis, Notarcha derogata, Plodia interpunctella, Ostrinia furnacalis, Ostrinia nubilaris, Hellula undalis, Pediasia teterrellus and the like,

Noctuidae such as Spodoptera litura, Spodoptera exigua, Pseudaletia separata, Mamestra brassicae, Agrotis Ipsilon, Plusia nigrisigna, Thoricoplusia spp., Heliothis spp., Helicoverpa spp. and the like,

Pieridae such as Pieris rapae and the like,

Tortricidae such as Adoxophyes spp., Grapholita molesta, Leguminivora glycinivorella, Matsumuraeses azukivora, Adoxophyes orana fasciata, Adoxophyes sp., Homona magnanima, Archips fuscocupreanus, Cydia pomonella and the like,

Gracillariidae such as Caloptilia theivora, Phyllonorycter ringoneella and the like,

Carposinidae such as Carposina niponensis and the like,

Lyonetiidae such as Lyonetia spp. and the like,

Lymantriidae such as Lymantria spp., Euproctis spp., and the like,

Yponomeutidae such as Plutella xylostella and the like,

Gelechiidae such as Pectinophora gossypiella, Phthorimaea operculella and the like,

Arctiidae such as Hyphantria cunea and the like,

Tineidae such as Tinea translucens, Tineola bisselliella and the like;

### Diptera:

Culex such as Culex pipiens pallens, Culex tritaeniorhynchus, Culex quinquefasciatus and the like,

Aedes spp. such as Aedes aegypti, Aedes albopictus and the like,

Anopheles spp. such as Anopheles sinensis and the like,

Chironomidae,

Muscidae such as Musca domestica, Muscina stabulans and the like,

Calliphoridae,

Sarcophagidae,

Fanniidae,

Anthomyiidae such as Delia platura, Delia antiqua and the like,

Tephritidae such as Dacus cucurbitae, Ceratitis capitata and the like,

Drosophilidae,

Psychodidae,

Simuliidae,

Tabanidae such as Tabanus trigonus and the like,

Stomoxyidae,

Agromyzidae such as Agromyza oryzae, Hydrellia griseola, Chlorops oryzae, Liriomyza trifolii, Liriomyza sativae and the like;

### Coleoptera:

Epilachna vigintioctopunctata, Aulacophora femoralis, Phyllotreta striolata, Oulema oryzae, Echinocnemus squameus, Lissorhoptrus oryzophilus, Anthonomus grandis, Callosobruchus chinensis, Sphenophorus venatus, Popillia japonica, Anomala cuprea, Diabrotica spp., Leptinotarsa decemlineata, Agriotes spp., Lasioderma serricorne, Anthrenus verbasci, Tribolium castaneum, Lyctus brunneus, Anoplophora malasiaca, Tomicus piniperdaand the like;

### Thysanoptera:

Thripidae such as Frankliniella occidentalis, Thrips parmi, Scirtothrips dorsalis, Thrips tabaci, Frankliniella intonsa, Frankliniella fuscaand the like;

### Hymenoptera:

Athalia rosae, Acromyrmex spp., Solenopsis spp. and the like;

### Blattodea:

Blattidae, Blattellidae and the like;

### Orthoptera:

Locusta migratoria, Gryllotalpa africana, Oxya yezoensis, Oxya japonica and the like;

### Aphaniptera:

Pulex irritans and the like;

### Anoplura:

Pediculus humanus and the like;

### Isoptera:

Termitidae and the like;

### Blattodea:

Blattella germanica, Periplaneta fuliginosa, Periplaneta americana, Periplaneta brunnea, Blatta orientalis and the like;

### Acarina:

Tetranychidae such as Tetranychus urticae, Tetranychus kanzawai, Panonychus citri, Panonychus ulmi, Oligonychus spp., and the like,

Eriophyidae such as Aculops pelekassi, Aculus schlechtendali, and the like,

Tarsonemidae such as Polyphagotarsonemus latus, and the like,

Tenuipalpidae,

Tuckerellidae,

Metastigmata such as Haemaphysalis longicornis, Haemaphysalis flava, Dermacentor taiwanicus, Ixodes ovatus, Ixodes persulcatus, Boophilus microplus, and the like,

Acaridae such as Tyrophagus putrescentiae, and the like,

Pyroglyphidae such as Dermatophagoides farinae, Dermatophagoides ptrenyssnus, and the like,

Cheyletidae such as Cheyletus eruditus, Cheyletus malaccensis, Cheyletus moorei, and the like, and

Dermanyssidae.

The present compound can be produced according to the following production methods.

### Production Method 1

Among the present compounds, a compound shown by formula (I-a) wherein G is hydrogen can be produced from a compound shown by formula (II) according to any of the following methods.

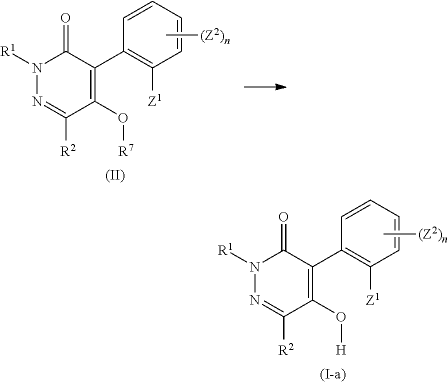

[wherein, R⁷ represents a C₁₋₆ alkyl group (e.g., a methyl group, an ethyl group, and the like), and R¹, R², Z¹, Z², and n represent the same meaning as described above.]

Production Method 1-1: Method of Reacting Compound Shown by Formula (II) with Alkali Metal Hydroxide

The reaction is generally carried out in a solvent. Examples of the solvent used in the reaction include, for example, water; ethers such as tetrahydrofuran and dioxane; and a mixture thereof.

The amount of the alkali metal hydroxide used in the reaction is generally at a rate of 1 to 120 mol and preferably at a rate of 1 to 40 mol, based on 1 mol of the compound shown by the formula (II).

The reaction temperature of the reaction is generally in the range from room temperature to the boiling point of the solvent to be used and preferably at the boiling point of the solvent. The reaction can be also performed in a sealed tube or a pressure resistant closed vessel. The reaction time of the reaction is generally from 5 minutes to a few weeks.

The progress of the reaction can be confirmed by analyzing a part of a reaction mixture by thin layer chromatography, high performance liquid chromatography, or the like.

After the completion of the reaction, the compound shown by the formula (I-a) can be isolated, for example, by neutralizing the reaction mixture, mixing the reaction mixture with water followed by extraction with an organic solvent, and carrying out the operations such as drying and concentrating the resulting organic layer.

Production Method 1-2: Method of Reacting Compound Shown by Formula (II) with Hydrobromic Acid

The reaction is generally carried out in a solvent. Examples of the solvent used in the reaction include, for example, water; ethers such as tetrahydrofuran and dioxane; organic acids such as acetic acid; and a mixture thereof.

The amount of the alkali metal hydroxide used in the reaction is generally at a rate of 1 to 120 mol and preferably at a rate of 1 to 40 mol, based on 1 mol of the compound shown by the formula (II).

The reaction temperature of the reaction is generally in the range from room temperature to the boiling point of the solvent to be used and preferably at the boiling point of the solvent. The reaction can be also performed in a sealed tube or a pressure resistant closed vessel. The reaction time of the reaction is generally from 5 minutes to a few weeks.

The progress of the reaction can be confirmed by analyzing a part of a reaction mixture by thin layer chromatography, high performance liquid chromatography, or the like.

After the completion of the reaction, the compound shown by the formula (I-a) can be isolated, for example, by neutralizing the reaction mixture, mixing the reaction mixture with water followed by extraction with an organic solvent, and carrying out the operations such as drying and concentrating the resulting organic layer.

Production Method 1-3: Method of Reacting Compound Shown by Formula (II) with Aluminum Chloride, Iodotrimethylsilane, or Boron Tribromide

The reaction is generally carried out in a solvent. Examples of the solvent used in the reaction include, for example, ethers such as tetrahydrofuran and dioxane; halogenated hydrocarbons such as dichloromethane, chloroform, and 1,2-dichloroethane; and a mixture thereof.

The amount of the alkali metal hydroxide used in the reaction is generally at a rate of 1 to 120 mol and preferably at a rate of 1 to 40 mol, based on 1 mol of the compound shown by the formula (II).

The reaction temperature of the reaction is generally in the range from room temperature to the boiling point of the solvent to be used and preferably at the boiling point of the solvent. The reaction can be also performed in a sealed tube or a pressure resistant closed vessel. The reaction time of the reaction is generally from 5 minutes to a few weeks.

The progress of the reaction can be confirmed by analyzing a part of a reaction mixture by thin layer chromatography, high performance liquid chromatography, or the like.

After the completion of the reaction, the compound shown by the formula (I-a) can be isolated, for example, by neutralizing the reaction mixture, mixing the reaction mixture with water followed by extraction with an organic solvent, and carrying out the operations such as drying and concentrating the resulting organic layer.

### Production Method 2

Among the present compounds, a compound shown by formula (I-b) wherein G is a group other than hydrogen can be produced from the compound shown by the formula (I-a) and a compound shown by formula (III).

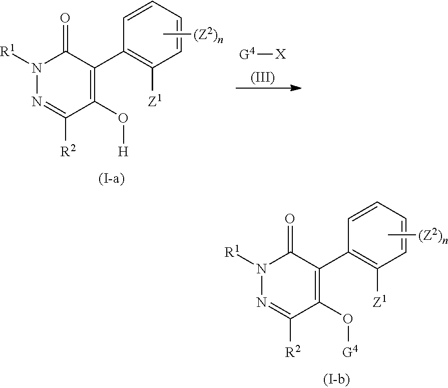

[wherein, G⁴ represents a group other than hydrogen among the definition of G, X represents halogen or a group represented by formula OG⁴, and R¹, R², Z¹, Z², and n represent the same meaning as described above.]

The reaction is carried out in a solvent. Examples of the solvent used in the reaction include, for example, aromatic hydrocarbons such as benzene, toluene, and xylene; ethers such as diethyl ether, diisopropyl ether, dioxane, tetrahydrofuran, and dimethoxyethane; halogenated hydrocarbons such as dichloromethane, chloroform, and 1,2-dichloroethane; ketones such as acetone and methyl ethyl ketone; nitriles such as acetonitrile; amides such as dimethylformamide and dimethylacetamide; sulfoxides such as dimethylsulfoxide; sulfones such as sulfolane; and a mixture thereof.

The amount of the compound shown by the formula (III) used in the reaction is generally at a rate of 1 mol or more and preferably at a rate of 1 to 3 mol, based on 1 mol of the compound shown by the formula (I-a).

The reaction is generally carried out in the presence of a base. Examples of the base used in the reaction include organic bases such as triethylamine, tripropylamine, pyridine, dimethylaminopyridine, and 1,8-diazabicyclo[5.4.0]-7-undecene; and inorganic bases such as sodium hydroxide, potassium hydroxide, calcium hydroxide, sodium carbonate, potassium carbonate, sodium hydrogen carbonate, calcium carbonate, and sodium hydride. The amount of the base used in the reaction is generally at a rate of 0.5 to 10 mol and preferably at a rate of 1 to 5 mol, based on 1 mol of the compound shown by the formula (I-a).

The reaction temperature of the reaction is generally from −30 to 180° C. and preferably from −10 to 50° C. The reaction time of the reaction is generally from 10 minutes to 30 hours.

The progress of the reaction can be confirmed by analyzing a part of a reaction mixture by thin layer chromatography, high performance liquid chromatography, or the like.

After the completion of the reaction, the compound shown by the formula (I-b) can be isolated, for example, by mixing the reaction mixture with water followed by extraction with an organic solvent, and carrying out the operations such as drying and concentrating the resulting organic layer.

### Production Method 3

The compound shown by the formula (I-a) can be produced by reacting a compound shown by formula (VI) with a base.

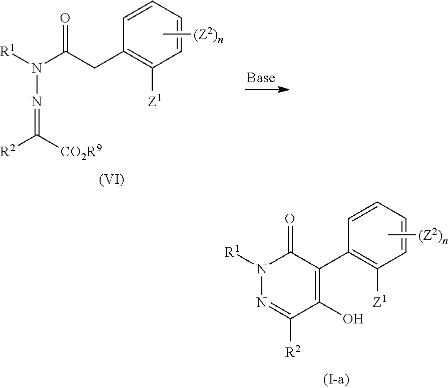

[wherein, R⁹ represents a C₁₋₆ alkyl group, and R¹, R², Z¹, Z², and n represent the same meaning as described above.]

The reaction is generally carried out in a solvent. Examples of the solvent used in the reaction include, for example, aromatic hydrocarbons such as benzene, toluene, and xylene; ethers such as diethyl ether, diisopropyl ether, dioxane, tetrahydrofuran, and dimethoxyethane; halogenated hydrocarbons such as dichloromethane, chloroform, and 1,2-dichloroethane; nitriles such as acetonitrile; amides such as dimethylformamide and dimethylacetamide; sulfones such as sulfolane; and a mixture thereof.

Examples of the base used in the reaction include, for example, metal alkoxides such as potassium tert-butoxide; alkali metal hydride such as sodium hydride; and organic bases such as triethylamine, tributylamine, and N,N-diisopropylethylamine. The amount of the base used in the reaction is generally at a rate of 1 to 10 mol and preferably at a rate of 2 to 5 mol, based on 1 mol of the compound shown by the formula (VI).

The reaction temperature of the reaction is generally from −60 to 180° C. and preferably from −10 to 100° C. The reaction time of the reaction is generally from 10 minutes to 30 hours.

The progress of the reaction can be confirmed by analyzing a part of a reaction mixture by thin layer chromatography, high performance liquid chromatography, or the like.

After the completion of the reaction, the compound shown by the formula (I-a) can be isolated, for example, by neutralizing the reaction mixture with the addition of an acid, mixing the reaction mixture with water followed by extraction with an organic solvent, and carrying out the operations such as drying and concentrating the resulting organic layer.

### Production Method 4

A compound shown by formula (I-a-x) can be produced according to the following production method.

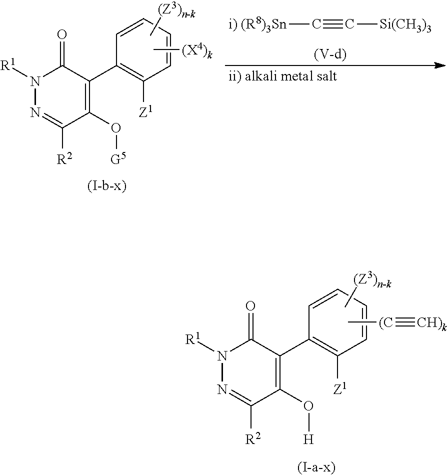

[wherein X⁴ represents halogen, R⁸ represents a C₂₋₆ alkyl group, Z³ represents a group other than the C₂₋₆ alkynyl group, halogen, the C₆₋₁₀ aryl group substituted with halogen, and the 5- or 6-membered heteroaryl group substituted with halogen, among the definition of Z² described above, G⁵ represents a C₂₋₆ alkylcarbonyl group or a C₂₋₆ alkyloxycarbonyl group, R¹, R², and Z¹ represent the same meaning as described above, n represents 1, 2, 3, or 4, and k is 1, 2, 3, or 4].

The production method has a first step of reacting a compound shown by formula (I-b-x) with an organic metal reagent shown by formula (V-d), followed by a second step of reacting with an alkali metal salt.

The reaction of the first step is carried out in a solvent. Examples of the solvent used in the reaction include, for example, aromatic hydrocarbons such as benzene, toluene, and xylene; ethers such as diethyl ether, diisopropyl ether, dioxane, tetrahydrofuran, and dimethoxyethane; halogenated hydrocarbons such as, chloroform and 1,2-dichloroethane; amides such as dimethylformamide and dimethylacetamide; and a mixture thereof.

In the reaction, the compound shown by the formula (V-d) is generally at a rate of k mol or more and preferably at a rate of k to 10 mol, based on 1 mol of the compound shown by the formula (I-b-x).

The reaction is generally carried out in the presence of a palladium catalyst such as tetrakis(triphenylphosphine)palladium and dichlorobis(triphenylphosphine)palladium. The amount of the catalyst used in the reaction is generally at a rate of 0.001 to 0.5 mol and preferably at a rate of 0.01 to 0.2 mol, based on 1 mol of the compound shown by the formula (I-b-x).

The reaction temperature of the reaction is generally from −80 to 180° C. and preferably from −30 to 150° C. The reaction time of the reaction is generally from 30 minutes to 100 hours.

The progress of the reaction can be confirmed by analyzing a part of a reaction mixture by thin layer chromatography, high performance liquid chromatography, or the like.

After the completion of the reaction, the product of the reaction can be isolated, for example, by concentrating the reaction mixture and carrying out the operations such as chromatographic purification.

The reaction of the second step is carried out in a solvent. Examples of the solvent used in the reaction include, for example, water; alcohols such as methanol and ethanol; ethers such as dioxane, tetrahydrofuran, and dimethoxyethane; and a mixture thereof.

Examples of the alkali metal salt used in the reaction include alkali metal hydroxide such as sodium hydroxide and potassium hydroxide; and alkali metal carbonate such as sodium carbonate and potassium carbonate. The amount of the alkali metal hydroxide used in the reaction is generally at a rate of (1+k) mol or more and preferably at a rate of 2 to 10 mol, based on 1 mol of the compound shown by the formula (I-b-x).

The reaction temperature of the reaction is generally from −30 to 180° C. and preferably from −10 to 50° C. The reaction time of the reaction is generally from 30 minutes to 100 hours.

The progress of the reaction can be confirmed by analyzing a part of a reaction mixture by thin layer chromatography, high performance liquid chromatography, or the like.

After the completion of the reaction, the compound shown by the formula (I-a-x) can be isolated, for example, by mixing the reaction mixture with water, neutralizing the reaction mixture with the addition of an acid, followed by extraction with an organic solvent, and carrying out the operations such as drying and concentrating the resulting organic layer.

### Production Method 5

A compound shown by formula (I-a-y) can be produced according to the following production method.

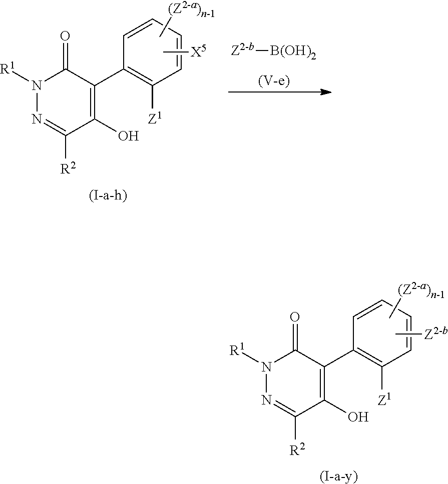

[wherein X⁵ represents halogen, Z^{2-a} represents a group other than halogen, the C₆₋₁₀ aryl group substituted with halogen, and the 5- or 6-membered heteroaryl group substituted with halogen, among the definition of Z² described above, Z^{2-b} represents a C₃₋₈ cycloalkyl group, a C₆₋₁₀ aryl group, or the 5- or 6-membered heteroaryl group (each may be substituted with at least one group selected from the group consisting of halogens and C₁₋₆ alkyl groups), R¹, R², and Z¹ represent the same meaning as described above, and n represents 1, 2, 3, or 4].

The compound shown by the formula (I-a-y) can be produced by reacting a compound shown by formula (I-a-h) with a compound shown by formula (V-e).

The amount of the compound shown by the formula (V-e) used in the reaction is generally at a rate of 1 mol or more and preferably at a rate of 1 to 3 mol, based on 1 mol of the compound shown by the formula (I-a-h).

The reaction is carried out in a solvent. Examples of the solvent used in the reaction include, for example, aromatic hydrocarbons such as benzene, toluene, and xylene; alcohols such as methanol, ethanol, and propanol; ethers such as diethyl ether, diisopropyl ether, dioxane, tetrahydrofuran, and dimethoxyethane; ketones such as acetone and methyl ethyl ketone; amides such as dimethylformamide and dimethylacetamide; sulfoxides such as dimethylsulfoxide; sulfones such as sulfolane; water; and a mixture thereof.

The reaction is carried out in the presence of a base. Examples of the base used in the reaction include, for example, organic bases such as triethylamine, tripropylamine, pyridine, dimethylaniline, dimethylaminopyridine, and 1,8-diazabicyclo[5.4.0]-7-undecene; and inorganic bases such as sodium hydroxide, potassium hydroxide, calcium hydroxide, sodium carbonate, potassium carbonate, sodium hydrogen carbonate, calcium carbonate, cesium carbonate, and potassium phosphate. The amount of the base used in the reaction is generally at a rate of 0.5 to 10 mol and preferably at a rate of 1 to 5 mol, based on 1 mol of the compound shown by the formula (I-a-h).

The reaction is generally carried out in the presence of a palladium catalyst such as tetrakis(triphenylphosphine)palladium, dichlorobis(triphenylphosphine)palladium, and dichlorobis (tricyclohexylphosphine)palladium. The amount of the catalyst in the reaction is generally at a rate of 0.001 to 0.5 mol and preferably at a rate of 0.01 to 0.2 mol, based on 1 mol of the compound shown by the formula (I-a-h).

The reaction temperature of the reaction is generally from 20 to 180° C. and preferably from 60 to 150° C. The reaction time of the reaction is generally from 30 minutes to 100 hours.

The progress of the reaction can be confirmed by analyzing a part of a reaction mixture by thin layer chromatography, high performance liquid chromatography, or the like.

After the completion of the reaction, the compound shown by the formula (I-a-y) can be isolated, for example, by mixing the reaction mixture with water, neutralizing the reaction mixture with the addition of an acid, followed by extraction with an organic solvent, and carrying out the operations such as drying and concentrating the resulting organic layer.

Next, specific example of the present composition is shown below.

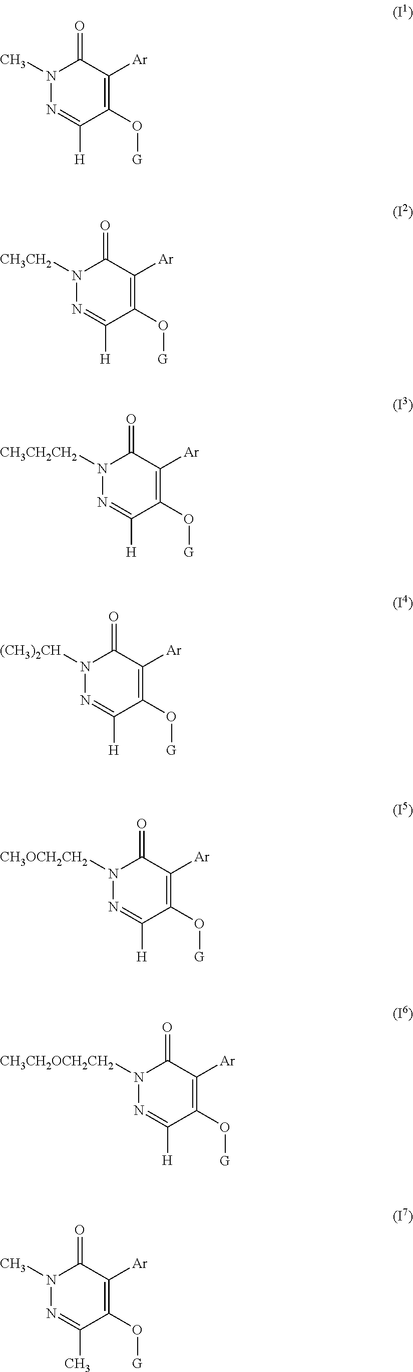

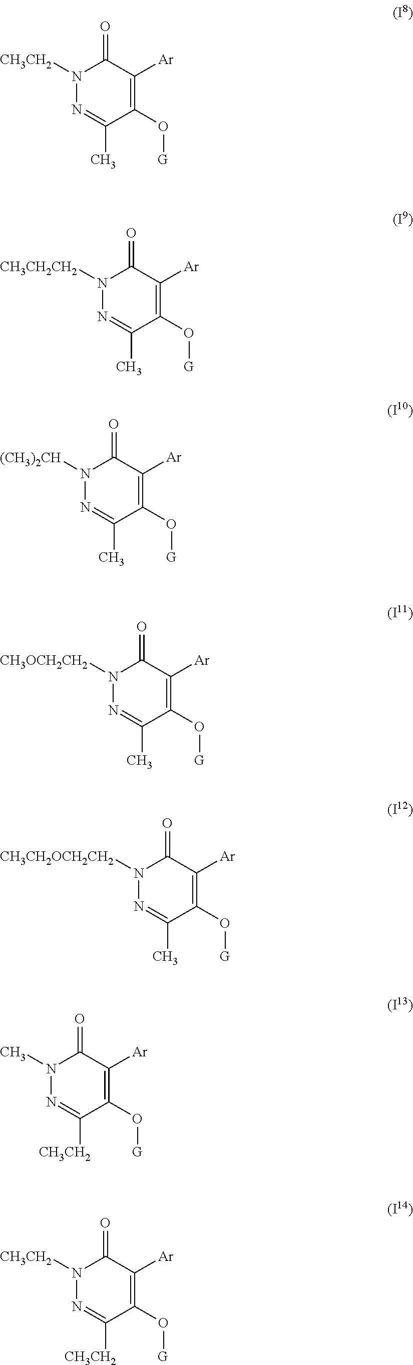

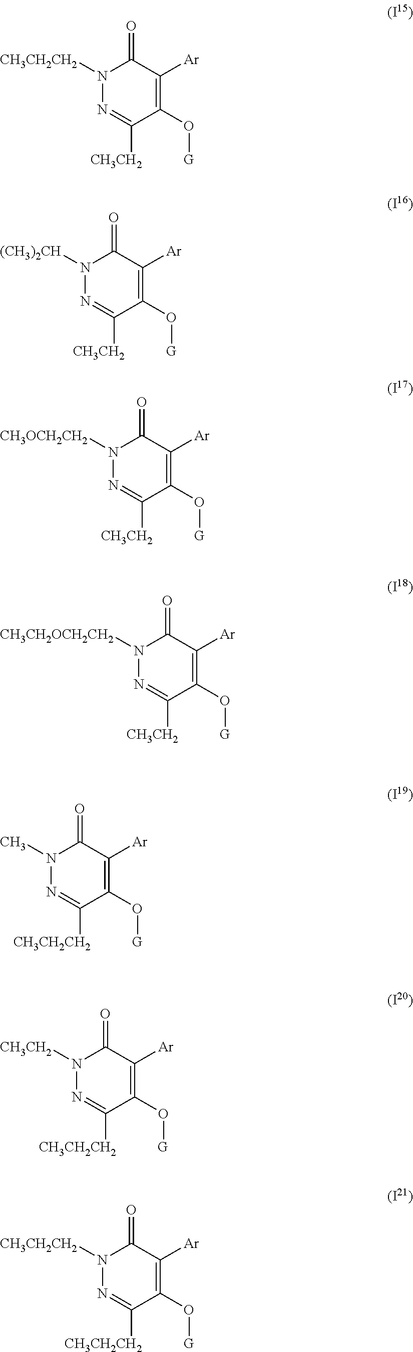

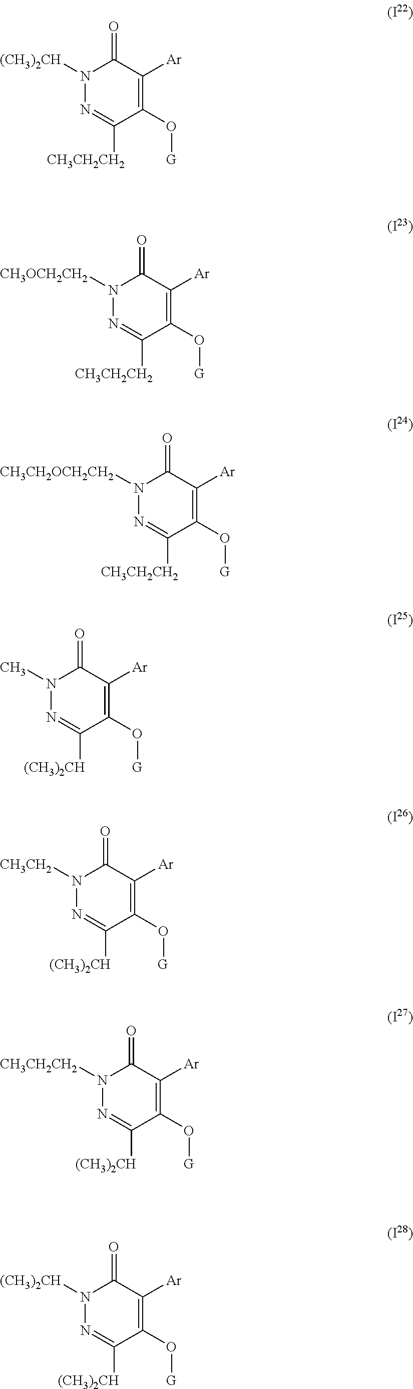

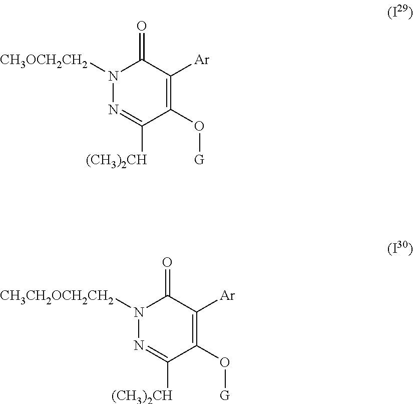

1) The pyridazinone compounds, wherein, in the formulae (I¹) to (I³⁰, Ar is a 2-ethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an alkyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

2) The pyridazinone compounds, wherein, in the formulae (I¹) to) (I³⁰), Ar is a 2-propylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

3) The pyridazinone compounds, wherein, in the formulae (I¹) to) (I³⁰), Ar is a 2,4-dimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

4) The pyridazinone compounds, wherein, in the formulae (I¹) to) (I³⁰), Ar is a 2,6-dimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

5) The pyridazinone compounds, wherein, in the formulae (I¹) to (I³⁰), Ar is a 2-ethyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

6) The pyridazinone compounds, wherein, in the formulae (I¹) to (I³⁰), Ar is a 2-ethyl-6-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

7) The pyridazinone compounds, wherein, in the formulae (I¹) to (I³⁰), Ar is a 2,6-diethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

8) The pyridazinone compounds, wherein, in the formulae (I¹) to (I³⁰), Ar is a 2,4,6-trimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

9) The pyridazinone compounds, wherein, in the formulae (I¹) to) (I³⁰), Ar is a 2-ethyl-4,6-dimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

10) The pyridazinone compounds, wherein, in the formulae (I¹) to (I³⁰), Ar is a 2,6-diethyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

11) The pyridazinone compounds, wherein, in the formulae (I¹) to (I³⁰), Ar is a 2,4,6-triethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

12) The pyridazinone compounds, wherein, in the formulae (I¹) to (I³⁰), Ar is a 2,4-diethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

13) The pyridazinone compounds, wherein, in the formulae (I¹) to (I³⁰), Ar is a 2,4-diethyl-6-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

14) The pyridazinone compounds, wherein, in the formulae (I¹) to (I³⁰), Ar is a 4-chloro-2,6-diethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

15) The pyridazinone compounds, wherein, in the formulae (I¹) to (I³⁰), Ar is a 4-bromo-2,6-diethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

16) The pyridazinone compounds, wherein, in the formulae (I¹) to (I³⁰), Ar is a 4-cyano-2,6-diethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

17) The pyridazinone compounds, wherein, in the formulae (I¹) to (I³⁰), Ar is a 2,6-diethyl-4-methoxyphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

18) The pyridazinone compounds, wherein, in the formulae (I¹) to (I³⁰), Ar is a 2,6-diethyl-4-nitrophenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

19) The pyridazinone compounds, wherein, in the formulae (I¹) to (I³⁰), Ar is a 2,6-diethyl-4-phenylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

20) The pyridazinone compounds, wherein, in the formulae (I¹) to (I³⁰), Ar is a 2,6-diethyl-4-ethynylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

21) The pyridazinone compounds, wherein, in the formulae (I¹) to (I³⁰), Ar is a 2-cyano-4,6-dimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

22) The pyridazinone compounds, wherein, in the formulae (I¹) to (I³⁰), Ar is a 2-cyano-6-ethyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

23) The pyridazinone compounds, wherein, in the formulae (I¹) to (I³⁰), Ar is a 2,4-dichloro-6-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

24) The pyridazinone compounds, wherein, in the formulae (I¹) to (I³⁰), Ar is a 2-chloro-4,6-dimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

25) The pyridazinone compounds, wherein, in the formulae (I¹) to (I³⁰), Ar is a 2-chloro-6-ethyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

26) The pyridazinone compounds, wherein, in the formulae (I¹) to (I³⁰), Ar is a 2,4-dichloro-6-ethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

27) The pyridazinone compounds, wherein, in the formulae (I¹) to (I³⁰), Ar is a 2-bromo-6-ethyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

28) The pyridazinone compounds, wherein, in the formulae (I¹) to (I³⁰), Ar is a 4-chloro-2-ethyl-6-methoxyphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

29) The pyridazinone compounds, wherein, in the formulae (I¹) to (I³⁰), Ar is a 2-ethyl-6-methoxy-4-methylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

30) The pyridazinone compounds, wherein, in the formulae (I¹) to (I³⁰), Ar is a 4-(4-chlorophenyl)-2,6-diethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

31) The pyridazinone compounds, wherein, in the formulae (I¹) to (I³⁰), Ar is a 2,6-diethyl-4-(4-methylphenyl)phenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

32) The pyridazinone compounds, wherein, in the formulae (I¹) to (I³⁰), Ar is a 2-ethyl-6-ethynyl-4-phenylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

33) The pyridazinone compounds, wherein, in the formulae (I¹) to (I³⁰), Ar is a 2-ethyl-6-methoxy-4-phenylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

34) The pyridazinone compounds, wherein, in the formulae (I¹) to (I³⁰), Ar is a 2-chloro-6-ethyl-4-phenylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

35) The pyridazinone compounds, wherein, in the formulae (I¹) to (I³⁰), Ar is a 2,6-diethyl-4-trifluoromethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

36) The pyridazinone compounds, wherein, in the formulae (I¹) to (I³⁰), Ar is a 2,6-diethyl-4-trifluoromethoxyphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

37) The pyridazinone compounds, wherein, in the formulae (I¹) to (I³⁰), Ar is a 2-ethyl-6-ethynyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

38) The pyridazinone compounds, wherein, in the formulae (I¹) to (I³⁰), Ar is a 2-chloro-6-ethyl-4-methoxyphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

39) The pyridazinone compounds, wherein, in the formulae (I¹) to (I³⁰), Ar is a 2-cyclopropyl-6-ethyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

40) The pyridazinone compounds, wherein, in the formulae (I¹) to (I³⁰), Ar is a 4-cyclopropyl-2,6-diethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

41) The pyridazinone compounds, wherein, in the formulae (I¹) to (I³⁰), Ar is a 5-(4-chlorophenyl)-2-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

42) The pyridazinone compounds, wherein, in the formulae (I¹) to (I³⁰), Ar is a 5-(4-fluorophenyl)-2-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

43) The pyridazinone compounds, wherein, in the formulae (I¹) to (I³⁰), Ar is a 2-bromo-4,6-dimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

44) The pyridazinone compounds, wherein, in the formulae (I¹) to (I³⁰), Ar is a 2-methoxy-4,6-dimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

45) The pyridazinone compounds, wherein, in the formulae (I¹) to (I³⁰), Ar is a 2-ethynyl-4,6-dimethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

46) The pyridazinone compounds, wherein, in the formulae (I¹) to (I³⁰), Ar is a 5-bromo-2-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

47) The pyridazinone compounds, wherein, in the formulae (I¹) to (I³⁰), Ar is a 3-bromo-2,6-diethyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

48) The pyridazinone compounds, wherein, in the formulae (I¹) to (I³⁰), Ar is a 3-(4-chlorophenyl)-2,6diethyl-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

49) The pyridazinone compounds, wherein, in the formulae (I¹) to (I³⁰), Ar is a 3-chloro-2,4,6-triethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

50) The pyridazinone compounds, wherein, in the formulae (I¹) to (I³⁰), Ar is a 3-bromo-2,4,6-triethylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

51) The pyridazinone compounds, wherein, in the formulae (I¹) to (I³⁰), Ar is a 5-(2-chlorophenyl)-2-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

52) The pyridazinone compounds, wherein, in the formulae (I¹) to (I³⁰), Ar is a 5-(3-chlorophenyl)-2-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

53) The pyridazinone compounds, wherein, in the formulae (I¹) to (I³⁰), Ar is a 5-(2-fluorophenyl)-2-methylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

54) The pyridazinone compounds, wherein, in the formulae (I¹) to (I³⁰), Ar is a 5-(3-fluorophenyl)-2-methylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

55) The pyridazinone compounds, wherein, in the formulae (I¹) to (I³⁰), Ar is a 2-methyl-5-phenylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

56) The pyridazinone compounds, wherein, in the formulae (I¹) to (I³⁰), Ar is a 2-methyl-5-(3-methylphenyl)phenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

57) The pyridazinone compounds, wherein, in the formulae (I¹) to (I³⁰), Ar is a 2-methyl-5-(4-methylphenyl)phenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

58) The pyridazinone compounds, wherein, in the formulae (I¹) to (I³⁰), Ar is a 5-(3,4-difluorophenyl)-2-methylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

59) The pyridazinone compounds, wherein, in the formulae (I¹) to (I³⁰), Ar is a 5-(4-chlorophenyl)-2-ethylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

60) The pyridazinone compounds, wherein, in the formulae (I¹) to (I³⁰), Ar is a 2,5-dimethylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

61) The pyridazinone compounds, wherein, in the formulae (I¹) to (I³⁰), Ar is a 4-(4-chlorophenyl)-2-methylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

62) The pyridazinone compounds, wherein, in the formulae (I¹) to (I³⁰), Ar is a 3-chloro-2,4,6-trimethylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

63) The pyridazinone compounds, wherein, in the formulae (I¹) to (I³⁰), Ar is a 2,6-diethyl-4-(3-pyridyl)phenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

64) The pyridazinone compounds, wherein, in the formulae (I¹) to (I³⁰), Ar is a 2,6-diethyl-4-(4-pyridyl)phenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

65) The pyridazinone compounds, wherein, in the formulae (I¹) to (I³⁰), Ar is a 2,6-diethyl-4-(3-thienyl)phenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

66) The pyridazinone compounds, wherein, in the formulae (I¹) to (I³⁰), Ar is a 2,6-diethyl-4-(3-furyl)phenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

67) The pyridazinone compounds, wherein, in the formulae (I¹) to (I³⁰), Ar is a 3,5-dichloro-2,4,6-triethylphenyl group, and G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

68) The pyridazinone compounds, wherein, in the formulae (I¹) to (I³⁰), Ar is a 2,4,6-triethyl-3-iodophenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

69) The pyridazinone compounds, wherein, in the formulae (I¹) to (I³⁰), Ar is a 2,4,6-triethyl-3-fluorophenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

70) The pyridazinone compounds, wherein, in the formulae (I¹) to (I³⁰), Ar is a 2,6-diethyl-3-fluoro-4-methylphenyl group, and

G is hydrogen, an acetyl group, a trifluoroacetyl group, a propionyl group, a butyryl group, an isobutyryl group, an isovaleryl group, a pivaloyl group, a 2,2-dimethylbutyryl group, a 3,3-dimethylbutyryl group, a cyclohexylcarbonyl group, a benzoyl group, a benzylcarbonyl group, a methoxycarbonyl group, an ethoxycarbonyl group, an isopropoxycarbonyl group, a tert-butoxycarbonyl group, a phenoxycarbonyl group, an allyloxycarbonyl group, a dimethylaminocarbonyl group, a dimethylaminothiocarbonyl group, a methanesulfonyl group, a trifluoromethanesulfonyl group, a benzenesulfonyl group, or a p-toluenesulfonyl group.

### Reference Production Method 1

The compound shown by the formula (II) can be produced, for example, according to the following method.

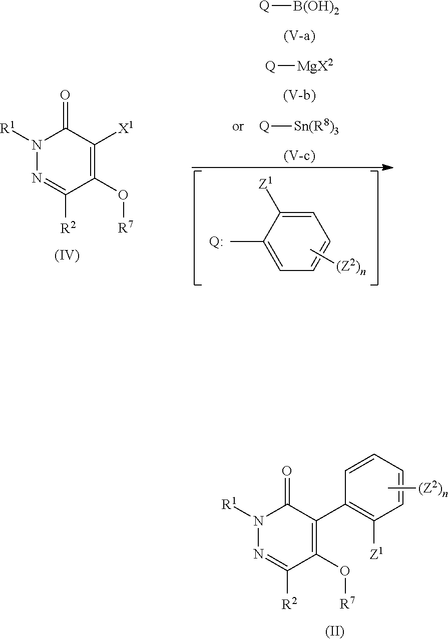

[wherein X¹ represents a leaving group (for example, halogen such as chlorine, bromine, or iodine), X² represents halogen, and R¹, R², R⁷, R⁸, Z¹, Z², and n represent the same meaning as described above.]

Reaction of Compound Shown by Formula (IV) with Compound Shown by Formula (V-a)

The reaction is carried out in a solvent. Examples of the solvent used in the reaction include aromatic hydrocarbons such as benzene, toluene, and xylene; alcohols such as methanol, ethanol, and propanol; ethers such as diethyl ether, diisopropyl ether, dioxane, tetrahydrofuran, and dimethoxyethane; ketones such as acetone and methyl ethyl ketone; amides such as dimethylformamide and dimethylacetamide; sulfoxides such as dimethylsulfoxide; sulfones such as sulfolane; water; and a mixture thereof.

The reaction is carried out in the presence of a base. Examples of the base used in the reaction include organic bases such as triethylamine, tripropylamine, pyridine, dimethylaniline, dimethylaminopyridine, and 1,8-diazabicyclo[5.4.0]-7-undecene; and inorganic bases such as sodium hydroxide, potassium hydroxide, calcium hydroxide, sodium carbonate, potassium carbonate, sodium hydrogen carbonate, calcium carbonate, cesium carbonate, and potassium phosphate. The amount of the base used in the reaction is generally at a rate of 0.5 to 10 mol and preferably at a rate of 1 to 5 mol, based on 1 mol of the compound shown by the formula (IV).

The reaction is generally carried out in the presence of a palladium catalyst such as tetrakis(triphenylphosphine)palladium and dichlorobis(triphenylphosphine)palladium. The amount of the catalyst used in the reaction is generally at a rate of 0.001 to 0.5 mol and preferably at a rate of 0.01 to 0.2 mol, based on 1 mol of the compound shown by the formula (IV). The reaction can be also performed further in the presence of a quaternary ammonium salt. Examples of the quaternary ammonium salt that can be used in the reaction include tetrabutylammonium bromide.

The reaction temperature of the reaction is generally from 20 to 180° C. and preferably from 60 to 150° C. The reaction time of the reaction is generally from 30 minutes to 100 hours.

The progress of the reaction can be confirmed by analyzing a part of a reaction mixture by thin layer chromatography, high performance liquid chromatography, or the like.

After the completion of the reaction, the compound shown by the formula (II) can be isolated, for example, by mixing the reaction mixture with water followed by extraction with an organic solvent, and carrying out the operations such as drying and concentrating the resulting organic layer.

Reaction of Compound Shown by Formula (IV) with Compound Shown by Formula (V-b)

The reaction is carried out in a solvent. Examples of the solvent used in the reaction include aromatic hydrocarbons such as benzene, toluene, and xylene; ethers such as diethyl ether, diisopropyl ether, dioxane, tetrahydrofuran, and dimethoxyethane; and a mixture thereof.

The reaction is generally carried out in the presence of a nickel catalyst such as dichlorobis(1,3-diphenylphosphino)propane nickel and dichlorobis(triphenylphosphine)nickel; a palladium catalyst such as tetrakis(triphenylphosphine)palladium and dichlorobis (triphenylphosphine)palladium. The amount of the catalyst used in the reaction is generally at a rate of 0.001 to 0.5 mol and preferably at a rate of 0.01 to 0.2 mol, based on 1 mol of the compound shown by the formula (IV).

The reaction temperature of the reaction is generally from −80 to 180° C. and preferably from −30 to 150° C. The reaction time of the reaction is generally from 30 minutes to 100 hours.

The progress of the reaction can be confirmed by analyzing a part of a reaction mixture by thin layer chromatography, high performance liquid chromatography, or the like.

After the completion of the reaction, the compound shown by the formula (II) can be isolated, for example, by mixing the reaction mixture with water followed by extraction with an organic solvent, and carrying out the operations such as drying and concentrating the resulting organic layer.

Reaction of Compound Shown by Formula (IV) with Compound Shown by Formula (V-c)

The reaction is carried out in a solvent. Examples of the solvent used in the reaction include aromatic hydrocarbons such as benzene, toluene, and xylene; ethers such as diethyl ether, diisopropyl ether, dioxane, tetrahydrofuran, and dimethoxyethane; halogenated hydrocarbons such as chloroform, and 1,2-dichloroethane; amides such as dimethylformamide and dimethylacetamide; and a mixture thereof.

The reaction is generally carried out in the presence of a palladium catalyst such as tetrakis(triphenylphosphine)palladium and dichlorobis(triphenylphosphine)palladium. The amount of the catalyst used in the reaction is generally at a rate of 0.001 to 0.5 mol and preferably at a rate of 0.01 to 0.2 mol, based on 1 mol of the compound shown by the formula (IV).

The reaction temperature of the reaction is generally from −80 to 180° C. and preferably from −30 to 150° C. The reaction time of the reaction is generally from 30 minutes to 100 hours.

The progress of the reaction can be confirmed by analyzing a part of a reaction mixture by thin layer chromatography, high performance liquid chromatography, or the like.

After the completion of the reaction, the compound shown by the formula (II) can be isolated, for example, by mixing the reaction mixture with water followed by extraction with an organic solvent, and carrying out the operations such as drying and concentrating the resulting organic layer.

### Reference Production Method 2

The compound shown by the formula (VI) can be produced, for example, by reacting a compound shown by formula (VII) with a compound shown by formula (VIII).

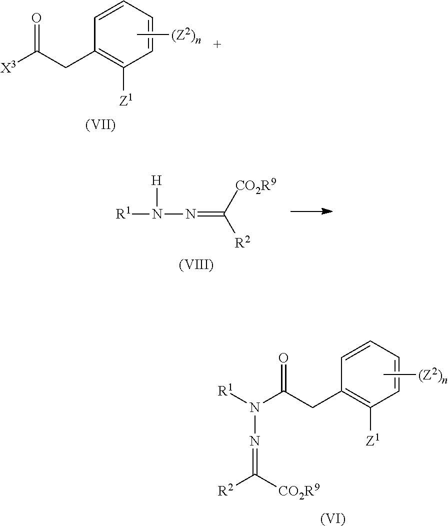

[wherein X³ represents halogen, and R¹, R², R⁹, Z¹, Z², and n represent the same meaning as described above.]

The reaction is generally carried out in a solvent. Examples of the solvent used in the reaction include nitriles such as acetonitrile; ketones such as acetone; aromatic hydrocarbons such as benzene, toluene, and xylene; ethers such as diethyl ether, diisopropyl ether, dioxane, tetrahydrofuran, and dimethoxyethane; halogenated hydrocarbons such as dichloromethane, chloroform, and 1,2-dichloroethane; amides such as dimethylformamide and dimethylacetamide; sulfones such as sulfolane; and a mixture thereof.

The reaction is generally carried out in the presence of a base. Examples of the base used in the reaction include organic bases such as triethylamine, tripropylamine, pyridine, dimethylaminopyridine, 1,8-diazabicyclo[5.4.0]-7-undecene, and 1,4-diazabicyclo[2.2.2] octane; and inorganic bases such as sodium hydroxide, potassium hydroxide, calcium hydroxide, sodium carbonate, potassium carbonate, sodium hydrogen carbonate, calcium carbonate, and sodium hydride.

The compound shown by the formula (VIII) is used in the reaction generally at a rate of 1 mol or more and preferably at a rate of 1 to 3 mol, based on 1 mol of the compound shown by the formula (VII). The base is used in the reaction generally at a rate of 0.5 to 10 mol and preferably at a rate of 1 to 5 mol, based on 1 mol of the compound shown by the formula (VII).

The reaction temperature of the reaction is generally from −30 to 180° C. and preferably from −10 to 50° C. The reaction time of the reaction is generally from 10 minutes to 30 hours.

The progress of the reaction can be confirmed by analyzing a part of a reaction mixture by thin layer chromatography, high performance liquid chromatography, or the like.

After the completion of the reaction, the compound shown by the formula (VI) can be isolated, for example, by mixing the reaction mixture with water followed by extraction with an organic solvent, and carrying out the operations such as drying and concentrating the resulting organic layer.

### Reference Production Method 3

The compound shown by the formula (VII) can be prepared by reacting a compound shown by formula (IX):

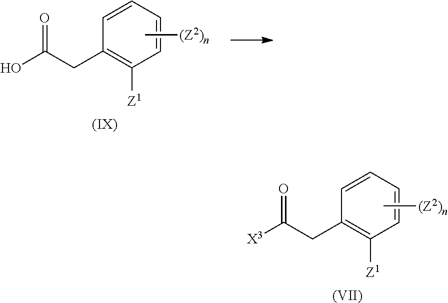

[wherein Z¹, Z², X³, and n represent the same meaning as described above.] with a halogenating agent (e.g., thionyl chloride, thionyl bromide, phosphorus oxychloride, oxalyl chloride, or the like).

### EXAMPLES

The present invention will be further specifically illustrated by the following production examples, formulation examples, and test examples, but the present invention is not limited to these examples.

### Production Example 1

To 3.193 g of 4-(2-ethylphenyl)-5-methoxy-2-methyl-3(2H)-pyridazinone [compound (II-1)] were added 50 mL of water, 4.657 g of potassium hydroxide (content 85%) and 5 mL of 1,4-dioxane. This mixture was heated under reflux for 36 hours. After cooling the reaction mixture, concentrated hydrochloric acid, 10 mL of water and 100 mL of ethyl acetate were added to the reaction mixture. An insoluble substance in this mixture was removed by filtration, and thereafter, the mixture was separated. The organic layer was washed with water, followed by saturated saline, and dried with anhydrous magnesium sulfate, and thereafter concentrated. The resulting solid was washed with a mixed solvent of ethyl acetate-hexane (1:2), to give 2.050 g of 4-(2-ethylphenyl)-5-hydroxy-2-methyl-3(2H)-pyridazinone [compound (I-a-1)] as a colorless crystal.

The compounds produced according to Production Example 1 are shown in Table 1 with the compound (1-a-1).

The compound shown by the formula (I-a):

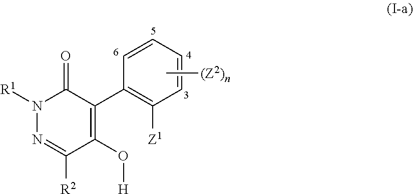

TABLE 1

| Compound | R¹ | R² | Z¹ | (Z²)ₙ | Melting Point/° C. |
|---|---|---|---|---|---|
| I-a-1 | Me | H | Et | — | 218-220 |
| I-a-2 | Et | H | Et | — | 190-192 |
| I-a-3 | i-Pr | H | Et | — | 226-227 |
| I-a-4 | MeOCH₂CH₂ | H | Et | — | 137-139 |
| I-a-5 | Me | H | Pr | — | 210-211 |
| I-a-6 | Me | H | Me | 6-Me | 267-271 |
| I-a-7 | Me | H | Et | 6-Me | 239-242 |
| I-a-8 | Me | H | Et | 6-Et | 247-249 |
| I-a-9 | Me | H | Me | 4-Me | 219-220 |
| I-a-10 | Me | H | Me | 4-Me, 6-Me | 272-275 |
| I-a-11 | Et | H | Me | 4-Me, 6-Me | >300 |
| I-a-12 | Me | H | Et | 4-Me, 6-Et | 254-255 |

### Production Example 2

### 4-(2,6-diethyl-4-methylphenyl)-5-hydroxy-2,6-dimethyl-3(2H)-pyridazinone [compound (I-a-14)]

To 13 mL of a tetrahydrofuran solution of potassium tert-butoxide (1 mol/L) was added dropwise 55 ml of a toluene solution of 1.9 g of 2-[2-(2,6-diethyl-4-methylphenylacetyl)-2-methylhydrazono]ethyl propanoate [compound (VI-2)] at room temperature over about 1 hour under nitrogen atmosphere. This mixture was stirred at room temperature for 30 minutes. The reaction mixture was concentrated. 30 mL of ice water was added to the residue, and the mixture was washed with tert-butylmethylether (20 mL×2). 1.6 g of 35% hydrochloric acid was added to the aqueous layer, and the mixture was extracted with ethyl acetate (20 mL×3). The organic layer was washed with saturated saline (20 mL×2), dried with anhydrous magnesium sulfate, and concentrated. The residue was applied to a silica gel column chromatography (ethyl acetate:hexane=1:3), to give 0.76 g of a solid. The solid was washed with cold hexane and dried, to give 0.59 g of 4-(2,6-diethyl-4-methylphenyl)-5-hydroxy-2,6-dimethyl-3(2H)-pyridazinone [compound (I-a-14)] as a white powder.

The compounds produced according to Production Example 2 are shown in Table 2 with the compound (I-a-14).

The compound shown by the formula (I-a):

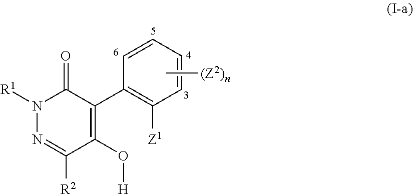

TABLE 2

| Compound | R¹ | R² | Z¹ | (Z²)ₙ | Melting Point/° C. |
|---|---|---|---|---|---|
| I-a-13 | Me | Me | Me | 4-Me, 6-Me | 199-201 |
| I-a-14 | Me | Me | Et | 4-Me, 6-Et | 205-206 |
| I-a-15 | Me | Me | Et | — | 171-172 |
| I-a-16 | Me | Me | Et | 4-Me | 187-188 |
| I-a-17 | Me | Me | Et | 4-Et, 6-Et | 188-190 |
| I-a-18 | Me | Me | Et | 4-Me, 6-Me | 176-177 |
| I-a-19 | Me | Et | Et | 4-Me, 6-Et | 194-195 |
| I-a-20 | Me | Et | Et | 4-Me | 148-149 |
| I-a-21 | Me | Et | Et | 4-Me, 6-Me | 188-189 |
| I-a-22 | Me | Et | Me | 4-Me, 6-Me | 210-211 |
| I-a-23 | Me | i-Pr | Et | 4-Me, 6-Et | 208-210 |
| I-a-24 | Me | Pr | Et | 4-Me, 6-Et | 175-176 |
| I-a-25 | Me | Et | Et | 4-Et, 6-Et | 170-171 |
| I-a-26 | Me | Pr | Et | 4-Et, 6-Et | 174-175 |
| I-a-27 | Me | Me | Et | 4-Et | 178-180 |
| I-a-28 | Me | Et | Et | 4-Et | 163-164 |
| I-a-29 | Me | Me | Et | 4-Et, 6-Me | 168-169 |
| I-a-30 | Me | Me | Et | 6-Et | 187-188 |
| I-a-60 | Et | Me | Et | 4-Me, 6-Et | 181-182 |

### Production Example 3

### 4-(4-chloro-2,6-diethylphenyl)-5-hydroxy-2,6-dimethyl-3(2H)-pyridazinone [compound (I-a-32)]

To 20 mL of a tetrahydrofuran solution of 0.55 g of potassium tert-butoxide was added dropwise 15 ml of a toluene solution of 0.79 g of 2-[2-(4-chloro-2,6-diethylphenylacetyl)-2-methylhydrazono]ethyl propanoate [compound (VI-22)] at 36 to 38° C. over about 20 minutes under nitrogen atmosphere. This mixture was stirred at 36 to 38° C. for 10 minutes. The reaction mixture was concentrated. 20 mL of ice water was added to the residue, and the mixture was washed with tert-butylmethylether (20 mL×2). 0.6 g of 35% hydrochloric acid was added to the aqueous layer, and the mixture was extracted with ethyl acetate (20 mL×2). The organic layer was washed with saturated saline (20 mL×2), dried with anhydrous magnesium sulfate, and concentrated. The resulting residue was applied to a silica gel column chromatography (ethyl acetate:hexane=1:4), to give 0.1 g of a solid. The solid was washed with a mixed solution of ethyl acetate-hexane (1:10) and dried, to give 0.07 g of 4-(4-chloro-2,6-diethylphenyl)-5-hydroxy-2,6-dimethyl-3(2H)-pyridazinone [compound (I-a-32)] as a white powder.

The compounds of the general formula (I) produced according to Production Example 3 are shown in Table 3 with the compound (I-a-32).

The compound shown by the formula (I-a):

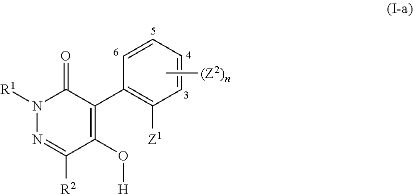

TABLE 3

| No. | R¹ | R² | Z¹ | (Z²)ₙ | Melting Point/° C. |
|---|---|---|---|---|---|
| I-a-31 | Me | Me | Et | 4-Ph, 6-Et | 231-232 |
| I-a-32 | Me | Me | Et | 4-Cl, 6-Et | 234-235 |
| I-a-33 | Me | Me | Et | 4-OMe, 6-Et | 205-206 |
| I-a-34 | Me | Me | Et | 4-Br, 6-Et | 251-252 |
| I-a-35 | Me | Me | Et | 4-CN, 6-Et | 243-245 |
| I-a-36 | Me | Me | Et | 4-CF₃, 6-Et | 270-271 |
| I-a-37 | Me | Me | Et | 4-OCF₃, 6-Et | 230-231 |
| I-a-38 | Me | Me | Et | 4-(c-Pr), 6-Et | 217-218 |
| I-a-39 | Me | Me | Et | 4-(4-Me—Ph), 6-Et | 214-215 |
| I-a-40 | Me | Me | Me | 5-(4-Cl—Ph) | 234-236 |
| I-a-41 | Me | Me | Me | 4-Me, 6-Br | 220-221 |
| I-a-42 | Me | Me | Me | 4-Me, 6-OMe | 164-166 |

### Production Example 4

To a mixture of 0.326 g of the compound (I-a-1), 12 mL of tetrahydrofuran and 0.40 mL of triethylamine was added 0.25 mL of benzoyl chloride under cooling with ice. This mixture was stirred under cooling with ice for 10 minute and at room temperature for 3 hours. 30 mL of water was added to the reaction mixture, and the mixture was extracted twice with 30 mL of ethyl acetate. The organic layer was washed with saturated saline, dried with anhydrous magnesium sulfate, and concentrated. The residue was applied to a silica gel column chromatography (ethyl acetate:hexane=1:2->2:1), to give 0.463 g of 5-benzoyloxy-4-(2-ethylphenyl)-2-methyl-3(2H)-pyridazinone [compound (I-b-1)] as a colorless oil.

The compounds produced according to Production Example 4 are shown in Table 4 with the compound (I-b-1).

The compound shown by the formula (I-b):

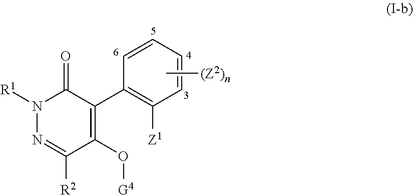

TABLE 4

| | | | | | | Melting |
|---|---|---|---|---|---|---|
| No. | R¹ | R² | Z¹ | (Z²)ₙ | G⁴ | Point/° C. |
| I-b-1 | Me | H | Et | — | COPh | * |
| I-b-2 | Me | H | Et | — | COMe | 69-70 |
| I-b-3 | Me | H | Et | — | COEt | * |
| I-b-4 | Me | H | Et | — | COi-Pr | 77-79 |
| I-b-5 | Me | H | Et | — | COt-Bu | 56-59 |
| I-b-6 | Me | H | Et | — | COc-Hex | * |
| I-b-7 | Me | H | Et | — | CO₂Me | 81-82 |
| I-b-8 | Me | H | Et | — | CONMe₂ | * |
| I-b-9 | Me | H | Et | — | SO₂Me | * |
| I-b-10 | Me | H | Pr | — | COMe | 78-79 |
| I-b-11 | Me | H | Me | 4-Me, 6-Me | COt-Bu | 93-96 |
| I-b-12 | Me | H | Et | 4-Me, 6-Et | COMe | 99-101 |
| I-b-13 | Me | Me | Me | 4-Me, 6-Me | COMe | 130-131 |
| I-b-14 | Me | Me | Et | 4-Me, 6-Et | COMe | 133-134 |
| I-b-15 | Me | Me | Et | 4-Me, 6-Et | COt-Bu | 105-106 |
| I-b-16 | Me | Me | Et | — | COMe | 148-149 |
| I-b-17 | Me | Me | Et | — | COt-Bu | 89 |
| I-b-18 | Me | Me | Et | 4-Me, 6-Et | CO₂Et | 73-74 |
| I-b-19 | Me | Me | Et | 4-Me, 6-Et | COPh | 145-146 |
| I-b-20 | Me | Me | Et | 4-Me | COMe | 142-143 |
| I-b-21 | Me | Me | Et | 4-Et, 6-Et | COMe | 103-104 |
| I-b-22 | Me | Me | Et | 4-Me, 6-Me | COMe | 106-107 |
| I-b-23 | Me | Me | Et | 4-Me, 6-Et | COEt | 103-104 |
| I-b-24 | Me | Me | Et | 4-Me, 6-Et | COi-Pr | 102-103 |
| I-b-25 | Me | Me | Et | 4-Me, 6-Et | CO₂Me | 95-96 |
| I-b-26 | Me | Me | Et | 4-Me, 6-Et | CO₂Ph | 105 |
| I-b-27 | Me | Me | Et | 4-Me, 6-Et | SO₂Me | 153-154 |
| I-b-28 | Me | Me | Et | 4-Me, 6-Et | SO₂CF₃ | 63-67 |
| I-b-29 | Me | Et | Et | 4-Me, 6-Et | COMe | 133-134 |
| I-b-30 | Me | Pr | Et | 4-Me, 6-Et | COMe | 161-162 |
| I-b-31 | Me | i-Pr | Et | 4-Me, 6-Et | COMe | 159-160 |
| I-b-32 | Me | Et | Et | 4-Et, 6-Et | COMe | 117-118 |
| I-b-33 | Me | Me | Et | 4-Et | COMe | 115-116 |
| I-b-34 | Me | Me | Et | 6-Et | COMe | 127-128 |
| I-b-35 | Me | Me | Et | 4-Me, 6-Me | CO₂Et | 65-67 |
| I-b-36 | Me | Me | Et | 4-Et, 6-Et | CO₂Me | 116-117 |
| I-b-37 | Me | Me | Et | 4-Me, 6-Me | CO₂Me | 95-96 |
| I-b-38 | Me | Me | Et | 4-Et, 6-Et | CO₂CH₂CH═CH₂ | 87-88 |
| I-b-39 | Me | Me | Et | 4-Me, 6-Et | CO₂CH₂CH═CH₂ | 62-63 |
| I-b-63 | Me | Me | Et | 4-Me, 6-Et | COCH2Cl | 142-143 |

Regarding the compounds marked with asterisk (*) in the column of melting point of Table 4, ¹H NMR data are shown below.

### Compound (I-b-1):

¹H-NMR (CDCl₃) δ ppm: 1.14 (3H, t, J=7.7 Hz), 2.45-2.62 (2H, m), 3.88 (3H, s), 7.09-7.12 (1H, m), 7.15-7.20 (1H, m), 7.28-7.30 (2H, m), 7.37-7.42 (2H, m), 7.55-7.60 (1H, m), 7.81-7.84 (2H, m), 7.95 (1H, s).

### Compound (I-b-3):

¹H-NMR (CDCl₃) δ ppm: 0.94 (3H, t, J=7.6 Hz), 1.13 (3H, t, J=7.7 Hz), 2.27 (2H, dq, J=1.4, 7.6 Hz), 2.38-2.56 (2H, m), 3.84 (3H, s), 7.00-7.03 (1H, m), 7.18-7.23 (1H, m), 7.30-7.35 (2H, m), 7.75 (1H, s).

### Compound (I-b-6):

¹H-NMR (CDCl₃) δ ppm: 1.13 (3H, t, J=7.7 Hz), 1.10-1.22 (5H, m), 1.5-1.7 (5H, m), 2.28 (1H, br.), 2.38-2.55 (2H, m), 3.84 (3H, s), 6.99-7.02 (1H, m), 7.17-7.22 (1H, m), 7.29-7.36 (2H, m), 7.72 (1H, s).

### Compound (I-b-8):

¹H-NMR (CDCl₃) δ ppm: 1.11 (3H, t, J=7.7 Hz), 2.40-2.57 (2H, m), 2.64 (3H, s), 2.85 (3H, s), 3.83 (3H, s), 7.05-7.08 (1H, m), 7.19-7.24 (1H, m), 7.30-7.36 (2H, m), 7.95 (1H, s).

### Compound (I-b-9):

¹H-NMR (CDCl₃) δ ppm: 1.18 (3H, t, J=7.6 Hz), 2.43-2.57 (2H, m), 2.58 (3H, s), 3.85 (3H, s), 7.16-7.19 (1H, m), 7.25-7.30 (1H, m), 7.36-7.43 (2H, m), 7.96 (1H, s).

### Production Example 5

To 5 mL of a tetrahydrofuran solution of 0.31 g of the compound (I-a-17) were added 96 mg of sodium hydride (60% in oil) and 0.39 mL of di-tert-butyl dicarbonate. This mixture was heated under reflux for 4.5 hours. 3 mL of 1 N hydrochloric acid was added to the reaction mixture, and the mixture was extracted with tert-butylmethylether (20 mL×2). The organic layer was washed with saturated saline (10 mL×2), dried with anhydrous magnesium sulfate, and concentrated. The residue was applied to a silica gel column chromatography (ethyl acetate:hexane=1:4), to give 0.41 g of 5-tert-butoxycarbonyloxy-2,6-dimethyl-4-(2,4,6-triethylphenyl)-3(2H)-pyridazinone [compound (1-b-40)] as a solid.

¹H-NMR (CDCl₃) δ ppm: 1.12 (6H, t, J=7.6 Hz), 1.24 (3H, t, J=7.6 Hz), 1.27 (9H, s), 2.30 (3H, s), 2.32-2.39 (4H, m), 2.63 (2H, q, J=7.6 Hz), 3.81 (3H, s), 6.96 (2H, s).

### Production Example 6

To 10 mL of a tetrahydrofuran solution of 0.04 g of the compound (I-a-31) were added 0.03 g of triethylamine, 0.02 g of acetic anhydride, and 0.002 g of 4-dimethylaminopyridine. This mixture was stirred at room temperature for 9.5 hours. The reaction mixture was concentrated. 10 mL of ice water was added to the residue, and the mixture was extracted with ethyl acetate (10 mL×3). The organic layer was washed with saturated saline (10 mL×2), dried with anhydrous magnesium sulfate, and concentrated. The residue was applied to a silica gel column chromatography (ethyl acetate:hexane=1:6), to give 0.05 g of a solid. The solid was washed with cooled hexane and dried, to give 0.025 g of 5-acetoxy-4-(2,6-diethyl-4-phenylphenyl)-2,6-dimethyl-3(2H)-pyridazinone [compound (I-b-41)] as a white powder.

The compounds of the general formula (I) produced according to Production Example 6 are shown in Table 5 with the compound (I-b-41).

The compound shown by the formula (I-b):

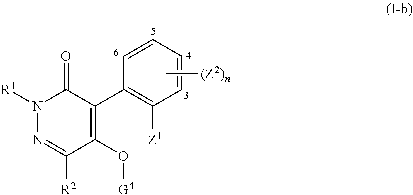

TABLE 5

| | | | | | | Melting |
|---|---|---|---|---|---|---|
| No. | R¹ | R² | Z¹ | (Z²)ₙ | G⁴ | Point/° C. |
| I-b-41 | Me | Me | Et | 4-Ph, 6-Et | COMe | 125-127 |
| I-b-42 | Me | Me | Et | 4-Br, 6-Et | CO₂Me | 125-127 |
| I-b-43 | Me | Me | Et | 4-(c-Pr), 6-Et | CO₂Me | 102-103 |
| I-b-44 | Me | Me | Et | 4-(4-Me—Ph), | COMe | 153-155 |
| | | | | 6-Et | | |
| I-b-45 | Me | Me | Me | 5-(4-Cl—Ph) | COMe | 153-154 |
| I-b-46 | Me | Me | Me | 4-Me, 6-Br | CO₂Me | 114-115 |
| I-b-47 | Me | Me | Me | 5-Br | COMe | 142-143 |
| I-b-48 | Me | Me | Me | 5-(4-F—Ph) | COMe | 127-128 |
| I-b-49 | Me | Me | Me | 5-(4-Cl—Ph) | CO₂Et | 106-108 |
| I-b-51 | Me | Me | Me | 5-(4-Cl—Ph) | COEt | 141-143 |
| I-b-52 | Me | Me | Me | 5-(4-Cl—Ph) | COi-Pr | 111-114 |
| I-b-53 | Me | Me | Me | 5-(4-Cl—Ph) | COt-Bu | 140-143 |
| I-b-54 | Me | Me | Me | 5-(4-Cl—Ph) | COCMe₂Et | 148-151 |
| I-b-55 | Me | Me | Me | 5-(4-Cl—Ph) | CO₂Me | 113-116 |
| I-b-56 | Me | Me | Me | 5-(4-Cl—Ph) | CO₂CH₂CH═CH₂ | 115-117 |
| I-b-57 | Me | Me | Me | 5-(4-Cl—Ph) | COCH₂t-Bu | 124-127 |
| I-b-58 | Me | Me | Me | 5-(4-Cl—Ph) | CO₂i-Pr | * |
| I-b-59 | Me | Me | Me | 5-(4-Cl—Ph) | CSNMe₂ | 182-185 |
| I-b-60 | Me | H | Et | 5-(4-Cl—Ph) | CO₂Me | * |
| I-b-61 | Me | H | Me | 5-(4-Cl—Ph) | CO₂Me | 114-116 |
| I-b-62 | Me | Me | Et | 5-(4-Cl—Ph) | CO₂Me | 152-154 |

Regarding the compounds marked with asterisk (*) in the column of melting point of Table 5, ¹H NMR data are shown below.

### Compound (I-b-58):

¹H-NMR (CDCl₃) δ ppm: 0.97-1.07 (6H, m), 2.23 (3H, s), 2.33 (3H, s), 3.83 (3H, s), 4.57 (1H, m), 7.23-7.39 (4H, m), 7.43-7.49 (3H, m).

### Compound (I-b-60):

¹H-NMR (CDCl₃) δ ppm: 1.17 (3H, t, J=7.8 Hz), 2.40-2.61 (2H, m), 3.75 (3H, s), 3.86 (3H, s), 7.25-7.28 (1H, m), 7.34-7.58 (6H, m), 7.88 (1H, s).

### Production Example 7

Nitrogen was blown into 10 ml of a toluene solution of 0.49 g of the compound (I-b-42) and 0.56 g of tributyl (trimethylsilylethynyl)tin. To this solution was added 0.08 g of tetrakis(triphenylphosphine)palladium, and the mixture was stirred at 100 to 110° C. under nitrogen atmosphere for 5 hours. The reaction mixture was cooled. This reaction mixture was filtered, and the filtrate was concentrated. The residue was applied to a silica gel column chromatography (ethyl acetate:hexane=1:8), to give 0.43 g of 4-(2,6-diethyl-4-trimethylsilylethynylphenyl)-5-methoxycarbonyloxy-2,6-dimethyl-3(2H)-pyridazinone as a yellow resin-like substance.

¹H-NMR (CDCl₃) δ ppm: 0.26 (9H, s), 1.12 (6H, t, J=7.6 Hz), 2.29 (3H, s), 2.26-2.43 (4H, m), 3.68 (3H, s), 3.81 (3H, s), 7.27 (2H, s).

0.4 g of 4-(2,6-diethyl-4-trimethylsilylethynylphenyl)-5-methoxycarbonyloxy-2,6-dimethyl-3(2H)-pyridazinone was dissolved in 15 mL of ethanol, and 2 mL of an aqueous solution of 0.13 g of 95.0% sodium hydroxide was added under cooling with ice. This mixture was stirred at room temperature for 22.5 hours. The reaction mixture was concentrated. 20 mL of ice water was added to the residue, and the mixture was washed with tert-butylmethylether (20 mL). 1.5 mL of 3 N hydrochloric acid was added to the aqueous layer, and the mixture was extracted with ethyl acetate (20 mL and 10 mL). The organic layer was washed with saturated saline (20 mL×2), dried with anhydrous magnesium sulfate, and concentrated. The residue was applied to a silica gel column chromatography (ethyl acetate:hexane=1:3), to give 0.15 g of a solid. The solid was washed with a mixed solution of ethyl acetate-hexane (1:10) and dried, to give 0.13 g of 4-(2,6-diethyl-4-ethynylphenyl)-5-hydroxy-2,6-dimethyl-3(2H)-pyridazinone [compound (I-a-43)] as a white powder. Melting Point of 216 to 217° C.

### Production Example 8

The same procedure as in the first half step of Production Example 7 was carried out, to give 4-(4,6-dimethyl-2-trimethylsilylethynylphenyl)-5-methoxycarbonyloxy-2,6-dimethyl-3(2H)-pyridazinone as a fawn solid from the compound (1-b-46).

¹H-NMR (CDCl₃) δ ppm: 0.07 (9H, s), 2.13 (3H, s), 2.29 (6H, s), 3.69 (3H, s), 3.81 (3H, s), 7.03 (1H, s), 7.19 (1H, s).

The same procedure as in the latter half step of Production Example 7 was carried out, to give 4-(2-ethynyl-4,6-dimethylphenyl)-5-hydroxy-2,6-dimethyl-3(2H)-pyridazinone [compound (I-a-44)] as a white powder from 4-(4,6-dimethyl-2-trimethylsilylethynylphenyl)-5-methoxycarbonyloxy-2,6-dimethyl-3(2H)-pyridazinone. Melting Point of 179 to 181° C.

### Production Example 9

1.8 g of potassium carbonate was added to 55 ml of a toluene solution of 2.8 g of 2-(methylhydrazono) ethyl propanoate. To the mixture was added dropwise a 20 ml of toluene solution of 3.4 g of 5-bromo-2-methylphenylacetyl chloride under cooling with ice over about 20 minutes. This mixture was stirred at room temperature for 3 hours. The reaction mixture was concentrated. 30 mL of ice water was added to the residue, and the mixture was extracted with ethyl acetate (20 mL, 10 mL×2). The organic layer was washed with saturated saline (20 mL×2), dried with anhydrous magnesium sulfate, and concentrated. The residue was applied to a basic alumina column chromatography (ethyl acetate:hexane=1:8), to give 3.0 g of 2-[2-(5-bromo-2-methylphenylacetyl)-2-methylhydrazono]ethyl propanoate as a yellow brown resin-like substance.

¹H-NMR (CDCl₃) δ ppm: 1.39 (3H, t, J=7.2 Hz), 2.23 (3H, s), 2.27 (3H, s), 3.37 (3H, s), 3.96 (2H, s), 4.35 (2H, q, J=7.2 Hz), 7.02 (1H, d, J=8.1 Hz), 7.25 (1H, br. d, J=8.3 Hz), 7.42 (1H, br. s).

To 80 mL of a tetrahydrofuran solution of 3.8 g of potassium tert-butoxide was added dropwise 60 ml of a toluene solution of 3.0 g of 2-[2-(5-bromo-2-methylphenylacetyl)-2-methylhydrazono]ethyl propanoate at 50 to 55° C. over about 60 minutes under nitrogen atmosphere. This mixture was stirred at 50 to 55° C. for 15 minutes. The reaction mixture was concentrated under reduced pressure. 50 mL of ice water was added to the residue, and the mixture was washed with tert-butylmethylether (30 mL×2). 3.7 g of 35% hydrochloric acid was added to the aqueous layer, and the mixture was extracted with ethyl acetate (30 mL×2). The organic layer was washed with saturated saline (30 mL×2), dried with anhydrous magnesium sulfate, and concentrated. The residue was applied to a silica gel column chromatography (ethyl acetate:hexane=1:1), to give 1.6 g of a solid. The solid was washed with a mixed solution of ethyl acetate-hexane (1:6) and dried, to give 1.5 g of 4-(5-bromo-2-methylphenyl)-5-hydroxy-2,6-dimethyl-3(2H)-pyridazinone [compound (I-a-45)] as a light brown powder. Melting Point of 217 to 218° C.

### Production Example 10

### 4-[5-(4-fluorophenyl)-2-methylphenyl]-5-hydroxy-2,6-dimethyl-3(2 H)-pyridazinone [compound (I-a-46)]

Nitrogen was blown into a mixture of 1.0 g of the compound (I-a-45), 0.64 g of 4-fluorophenylboronic acid, 0.31 g of sodium carbonate, 30 mL of dimethoxyethane and 8 mL of water at room temperature for 60 minutes. To this mixture was added 0.3 g of tetrakis(triphenylphosphine)palladium. This mixture was stirred at 90 to 100° C. for 10 hours. The reaction mixture was cooled and then filtered. The filtrate was concentrated. 20 mL of ice water and 1.0 g of 35% hydrochloric acid were added to the resulting residue, and the mixture was extracted with ethyl acetate (20 mL×2). The organic layer was washed with saturated saline (20 mL×2), dried with anhydrous magnesium sulfate, and concentrated. The residue was applied to a silica gel column chromatography (ethyl acetate:hexane=1:1), to give 0.33 g of a solid. The solid was washed with a mixed solution of ethyl acetate-hexane (1:6) and dried, to give 0.25 g of 4-[5-(4-fluorophenyl)-2-methylphenyl]-5-hydroxy-2,6-dimethyl-3(2 H)-pyridazinone [compound (I-a-46)] as a white powder. Melting Point of 216 to 217° C.

The compounds of the general formula (I) produced according to Production Example 10 are shown in Table 6 with the compound (I-a-46).

The compound shown by the formula (I-a):

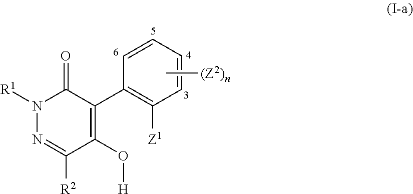

TABLE 6

| No. | R¹ | R² | Z¹ | (Z²)ₙ | Melting Point/° C. |
|---|---|---|---|---|---|
| I-a-46 | Me | Me | Me | 5-(4-F—Ph) | 216-217 |
| I-a-51 | Me | Me | Me | 5-(3-Cl—Ph) | 203-205 |
| I-a-52 | Me | Me | Me | 5-(2-Cl—Ph) | 218-220 |
| I-a-53 | Me | Me | Me | 5-(2-F—Ph) | 195-200 |
| I-a-54 | Me | Me | Me | 5-(3-F—Ph) | 194-196 |
| I-a-55 | Me | Me | Me | 5-Ph | 189-192 |
| I-a-56 | Me | Me | Me | 5-(4-Me—Ph) | 206-208 |
| I-a-57 | Me | Me | Me | 5-(3-Me—Ph) | 172-174 |
| I-a-58 | Me | Me | Me | 5-(3,4-F₂—Ph) | 198-200 |

### Production Example 11

To an acetic acid solution (2 mL) of 0.35 g of 4-[5-(4-chlorophenyl)-2-methylphenyl]-5-methoxy-2-methyl-3(2H)-pyridazin one was added 47% hydrobromic acid (0.6 mL). This mixture was stirred at 100° C. for 16 hours. After cooling the reaction mixture, water (50 mL) was added to the reaction mixture. The precipitated crystal was collected by filtration. This crystal was washed with water and dried, to give 0.28 g of 4-[5-(4-chlorophenyl)-2-methylphenyl]-5-hydroxy-2-methyl-3(2H)-pyridazinone [compound (I-a-59)] as a white powder. Melting Point of 250° C. or more.

¹H-NMR (CDCl₃) δ ppm: 2.22 (3H, s), 3.81 (3H, s), 5.73 (1H, br, s), 7.32-7.57 (7H, m), 7.86 (1H, s).

The compounds of the general formula (I) produced according to Production Example 11 are shown in Table 7 with the compound (I-a-59).

The compound shown by the formula (I-a):

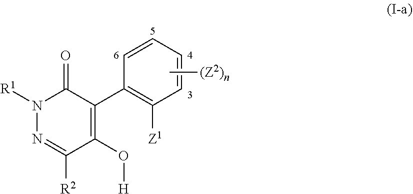

TABLE 7

| No. | R¹ | R² | Z¹ | (Z²)ₙ | Melting Point/° C. |
|---|---|---|---|---|---|
| I-a-59 | Me | H | Me | 5-(4-Cl—Ph) | >250 |
| I-a-61 | Me | H | Et | 5-(4-Cl—Ph) | >230 |
| I-a-62 | Me | Me | Me | 5-Me | 234-236 |
| I-a-63 | Me | Me | Me | 4-(4-Cl—Ph) | >240 |
| I-a-64 | Me | Me | Et | 5-(4-Cl—Ph) | 212-214 |
| I-a-65 | Me | H | Me | 4-Me, 5-Cl, 6-Me | >230 |
| I-a-75 | Me | Me | Me | 4-Me, 5-Cl, 6-Me | 227-228 |

### Production Example 12

To 10 mL of an acetic acid solution of 0.60 g of the compound (I-b-25) and 0.17 g of sodium acetate was added 1 mL of an acetic acid solution of 0.10 mL of bromine at room temperature. This mixture was stirred at room temperature for 3.5 hours and stirred at 50° C. for 2 hours. The reaction mixture was concentrated. 20 mL of ice water was added to the residue, and sodium hydrogen carbonate was added, thereafter the mixture was extracted with ethyl acetate (20 mL×2). The organic layer was washed with saturated saline (20 mL×2), dried with anhydrous magnesium sulfate, and then concentrated. The residue was applied to a silica gel column chromatography (ethyl acetate:hexane=1:10), to give 0.35 g of a solid. The solid was washed with cold hexane and air-dried, to give 0.30 g of 4-(3-bromo-2,6-diethyl-4-methylphenyl)-5-methoxycarbonyloxy-2,6-dimethyl-3(2H)-pyridazinone [compound (1-b-50)] as a white powder. Melting Point of 113 to 115° C.

¹H-NMR (CDCl₃) δ ppm: 1.04 (3H, t, J=7.6 Hz), 1.10 (3H, t, J=7.6 Hz), 2.21-2.35 (2H, m), 2.45-2.62 (2H, m), 2.30 (3H, s), 2.43 (3H, s), 3.74 (3H, s), 3.82 (3H, s), 7.05 (1H, s).

### Production Example 13

To 10 mL of a methanol solution of 0.20 g of the compound (I-b-50) was added 1 mL of an aqueous solution of 0.06 g of 95.0% sodium hydroxide at room temperature. This mixture was stirred at room temperature for 8.5 hours. The reaction mixture was concentrated. 5 mL of ice water was added to the residue, and 1 mL of 3 N hydrochloric acid was added. The precipitated solid was collected by filtration. This solid was washed with water and dried, to give 0.2 g of a solid. The solid was applied to a silica gel column chromatography (ethyl acetate:hexane=1:3), to give 0.18 g of a solid. The solid was washed with a mixed solution of ethyl acetate-hexane (1:10) and dried, to give 0.14 g of 4-(3-bromo-2,6-diethyl-4-methylphenyl)-5-hydroxy-2,6-dimethyl-3(2H)-pyridazinone [compound (I-a-47)] as a white powder. Melting Point of 235 to 236° C.

¹H-NMR (CDCl₃) δ ppm: 1.00 (3H, t, J=7.6 Hz), 1.06 (3H, t, J=7.6 Hz), 2.16-2.38 (2H, m), 2.47-2.67 (2H, m), 2.33 (3H, s), 2.45 (3H, s), 3.76 (3H, s), 5.27 (1H, s), 7.12 (1H, s).

### Production Example 14

The same procedure as in Production Example 10 was carried out, to give 4-[3-(4-chlorophenyl)-2,6-diethyl-4-methylphenyl)-5-hydroxy-2,6-dimethyl-3(2H)-pyridazinone [compound (I-a-48)] as a solid from the compound (1-a-47) and 4-chlorophenylboronic acid. Melting Point of 248 to 250° C.

### Production Example 15

To 10 mL of a chloroform solution of 0.50 g of the compound (1-a-17) were added 0.27 g of N-chlorosuccinimide and several milligrams of azobisisobutyronitrile. This mixture was heated under reflux for 12.5 hours. After cooling the reaction mixture to room temperature, the reaction mixture was concentrated. The residue was applied to a silica gel column chromatography (ethyl acetate:hexane=1:2), to give 0.46 g of 4-(3-chloro-2,4,6-triethylphenyl)-5-hydroxy-2,6-dimethyl-3(2H)-pyridazinone [compound (I-a-49)] as a solid. Melting Point of 225 to 226° C.

¹H-NMR (CDCl₃) δ ppm: 1.01 (3H, t, J=7.6 Hz), 1.07 (3H, t, J=7.6 Hz), 1.27 (3H, t, J=7.6 Hz), 2.22-2.40 (2H, m), 2.34 (3H, s), 2.46-2.62 (2H, m), 2.77-2.83 (2H, m), 3.77 (3H, s), 5.14 (1H, s), 7.12 (1H, s).

### Production Example 16

The same procedure as in Production Example 15 was carried out using N-bromosuccinimide in place of N-chlorosuccinimide, to give 4-(3-bromo-2,4,6-triethylphenyl)-5-hydroxy-2,6-dimethyl-3(2H)-pyridazinone [compound (I-a-50)] as a solid from the compound (I-a-17). Melting Point of 225 to 226° C.

### Production Example 17

The same procedure as in Production Example 10 was carried out, to give 4-[2,6-d]ethyl-4-(4-pyridyl)phenyl]-5-hydroxy-2,6-dimethyl-3(2H)-pyridazinone [compound (I-a-66)] from the compound (I-a-34) produced according to Production Example 3 and 4-pyridylboronic acid.

The compounds of the formula (I) produced according to Production Example 17 are shown in Table 8 with the compound (I-a-66).

The compound shown by the formula (I-a):

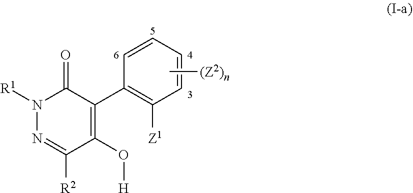

TABLE 8

| No. | R¹ | R² | Z¹ | (Z²)ₙ | Melting Point/° C. |
|---|---|---|---|---|---|
| I-a-66 | Me | Me | Et | 4-(4-pyridyl), 6-Et | 274-276 |
| I-a-67 | Me | Me | Et | 4-(3-thienyl), 6-Et | 242-243 |
| I-a-68 | Me | Me | Et | 4-(3-furyl), 6-Et | 185-187 |
| I-a-69 | Me | Me | Et | 4-(3-pyridyl), 6-Et | 268-271 |
| I-a-70 | Me | Me | Et | 4-(4-Cl—Ph), 6-Et | 206-208 |

### Production Example 18

To 5 ml of a toluene solution of 0.30 g of the compound (I-a-17) was added 0.16 mL of sulfuryl chloride. This mixture was heated under reflux for 1.5 hours. The reaction mixture was concentrated. The residue was applied to a silica gel column chromatography (ethyl acetate:hexane=1:3), to give 70 mg of 4-(3,5-dichloro-2,4,6-triethylphenyl)-5-hydroxy-2,6-dimethyl-3(2H)-pyridazinone [compound (I-a-71)] as a solid. Melting Point of 266 to 267° C.

### Production Example 19

To 5 mL of an acetonitrile solution of 0.30 g of the compound (I-a-17) were added 560 mg of N-iodosuccinimide and a drop of trifluoroacetic acid. This mixture was stirred at room temperature for 2 hours and further heated under reflux for 8 hours. An aqueous solution of saturated sodium thiosulfate (10 mL) was added to the reaction mixture, and the mixture was extracted with chloroform (10 mL×2). The organic layer was dried with anhydrous magnesium sulfate and concentrated. The residue was applied to a silica gel column chromatography (ethyl acetate:hexane=1:2), to give 0.26 g of 5-hydroxy-2,6-dimethyl-4-(2,4,6-triethyl-3-iodophenyl)-3(2H)-pyridazinone [compound (I-a-72)] as a solid. Melting Point of 217 to 219° C.

### Production Example 20

To 5 mL of an acetonitrile solution of 0.38 g of the compound (I-a-17) was added 1.34 g of 1-chloromethyl-4-fluoro-1,4-diazoniabicyclo[2.2.2]octane bis(tetrafluoroborate) (Selectfluor (trademark)). This mixture was stirred at 55° C. for 5 hours. Water (10 mL) was added to the reaction mixture, and the mixture was extracted with chloroform (10 mL×2). The organic layer was dried with sodium sulfate and concentrated. The residue was applied to a silica gel column chromatography (methanol:chloroform=1:20), to give 0.10 g of 5-hydroxy-2,6-dimethyl-4-(2,4,6-triethyl-3-fluorophenyl)-3(2H)-pyridazinone [compound (I-a-73)] as a solid.

¹H-NMR (CDCl₃) δ ppm: 1.01 (3H, t, J=7.6 Hz), 1.06 (3H, t, J=7.6 Hz), 1.26 (3H, t, J=7.4 Hz), 2.22-2.45 (4H, m), 2.33 (3H, s), 2.69 (2H, q, J=7.6 Hz), 3.76 (3H, s), 5.35 (1H, s) 7.05 (2H, d, J=7.6 Hz).

### Production Example 21

To 10 mL of an acetonitrile solution of 0.50 g of the compound (I-a-14) was added 560 mg of 1-chloromethyl-4-fluoro-1,4-diazoniabicyclo[2.2.2]octane bis(tetrafluoroborate) (Selectfluor (trademark)). This mixture was stirred at 55° C. for 5 hours. To this mixture was added 930 mg of Selectfluor (trademark), and further stirred at 55° C. for 18 hours. The reaction mixture was concentrated. Water (10 mL) was added to the residue, and the mixture was extracted with chloroform (20 mL×2). The organic layer was dried with anhydrous magnesium sulfate and concentrated. The residue was applied to a silica gel column chromatography (ethyl acetate:hexane=1:3), to give 0.31 g of 4-(2,6-diethyl-3-fluoro-4-methylphenyl)-5-hydroxy-2,6-dimethyl-3(2H)-pyridazinone [compound (I-a-74)] as a white solid.

Melting Point of 193 to 195° C.

Representative production examples of the compounds shown by the formula (II) are shown in Reference Example 1.

### Reference Example 1

To a mixture of 2.516 g of 4-chloro-5-methoxy-2-methyl-3(2H)-pyridazinone, 2.575 g of 2-ethylphenylboronic acid and 3.333 g of sodium carbonate were added 30 mL of 1,4-dioxane, 20 mL of water, 2.417 g of tetrabutylammonium bromide and 0.657 g of tetrakis(triphenylphosphine)palladium. This mixture was heated under reflux for 17 hours under nitrogen atmosphere. The reaction mixture was cooled. 50 mL of water was added to the reaction mixture, and the mixture was extracted with ethyl acetate. The organic layer was washed with saturated saline, dried with anhydrous magnesium sulfate, and concentrated. The resulting solid was washed with a mixed solvent of ethyl acetate-hexane (1:2), to give 3.238 g of 4-(2-ethylphenyl)-5-methoxy-2-methyl-3(2H)-pyridazinone [compound (II-1)] as a yellow crystal.

The compounds of the formula (II) produced according to Reference Example 1 are shown in Table 9 with the compound (II-1).

The compounds shown by the formula (II):

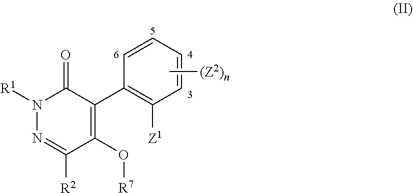

TABLE 9

| | | | | | | Melting |
|---|---|---|---|---|---|---|
| No. | R¹ | R² | Z¹ | (Z²)ₙ | R⁷ | Point/° C. |
| II-1 | Me | H | Et | — | Me | 127-130 |
| II-2 | Et | H | Et | — | Me | * |
| II-3 | i-Pr | H | Et | — | Me | 121-123 |
| II-4 | MeOCH₂CH₂ | H | Et | — | Me | * |
| II-5 | Me | H | Pr | — | Me | 86-88 |
| II-6 | Me | H | Me | 6-Me | Me | 187-189 |
| II-7 | Me | H | Et | 6-Me | Me | * |
| II-8 | Me | H | Et | 6-Et | Me | 165-166 |
| II-9 | Me | H | Me | 4-Me | Me | 141-142 |
| II-10 | Me | H | Me | 4-Me, 6-Me | Me | 186-192 |
| II-11 | Et | H | Me | 4-Me, 6-Me | Me | 100-102 |
| II-12 | Me | H | Et | 4-Me, 6-Et | Me | 147-149 |
| II-13 | Me | H | Me | 5-(4-Cl—Ph) | Me | * |
| II-14 | Me | H | Et | 5-(4-Cl—Ph) | Me | * |

Regarding the compounds marked with asterisk (*) in the column of melting point of Table 9, ¹H NMR data are shown below.

### Compound (II-2):

¹H-NMR (CDCl₃) δ ppm: 1.12 (3H, t, J=7.7 Hz), 1.39 (3H, t, J=7.3 Hz), 2.40-2.53 (2H, m), 3.81 (3H, s), 4.19-4.30 (2H, m), 7.10 (1H, d, J=7.6 Hz), 7.21-7.26 (1H, m), 7.30-7.33 (2H, m), 7.88 (1H, s).

### Compound (II-4):

¹H-NMR (CDCl₃) δ ppm: 1.12 (3H, t, J=7.7 Hz), 2.38-2.52 (2H, m), 3.38 (3H, s), 3.82 (3H, s), 3.77-3.84 (2H, m), 4.40 (2H, t, J=5.6 Hz), 7.11 (1H, d, J=7.6 Hz), 7.21-7.26 (1H, m), 7.30-7.34 (2H, m), 7.90 (1H, s).

### Compound (II-7):

¹H-NMR (CDCl₃) δ ppm: 1.08 (3H, t, J=7.7 Hz), 2.07 (3H, s), 2.30-2.45 (2H, m), 3.81 (3H, s), 3.82 (3H, s), 7.10 (1H, d, J=7.6 Hz), 7.13 (1H, d, J=7.6 Hz), 7.24 (1H, t, J=7.6 Hz), 7.85 (1H, s).

### Compound (II-13):

¹H-NMR (CDCl₃) δ ppm: 2.19 (3H, s), 3.83 (3H, s), 3.86 (3H, s), 7.31-7.53 (7H, m), 7.88 (1H, s).

### Compound (II-14):

¹H-NMR (CDCl₃) δ ppm: 1.16 (3H, t, J=7.8 Hz) 2.42-2.57 (2H, m), 3.83 (3H, s), 3.86 (3H, s), 7.27-7.42 (4H, m), 7.48-7.55 (3H, m), 7.88 (1H, s).

Representative production examples of the compounds shown by the formula (V-a) are shown in Reference Example 2.

### Reference Example 2

To 15.5 mL of butyllithium (1.6 mol/L hexane solution) was added dropwise 4.412 g of 2-propylbromobenzene dissolved in 45 mL of tetrahydrofuran at −70° C. over 85 minutes under nitrogen atmosphere. This mixture was stirred at −70° C. for 30 minutes. Subsequently, 3.75 mL of trimethyl borate was added dropwise to this mixture at −70° C. for 15 minutes. This mixture was stirred at −70° C. for 1 hour and stirred at room temperature for 18 hours. 33 mL of 2 N hydrochloric acid was added to the reaction mixture over 10 minutes. This mixture was stirred at room temperature for 4 hours. 20 mL of water was added to the reaction mixture, and the mixture was extracted with 70 mL of ethyl acetate. The organic layer was washed with saturated saline and dried with anhydrous magnesium sulfate, and the solvent was distilled away. The residue was applied to a silica gel column chromatography (ethyl acetate:hexane=1:2→2:1), to give 1.641 g of 2-propylphenylboronic acid as a colorless crystal.

¹H-NMR (CDCl₃) δ ppm: 1.01 (3H, t, J=7.4 Hz), 1.69-1.79 (2H, m), 3.15-3.20 (2H, m), 4.0-6.0 (2H, br.), 7.28-7.33 (2H, m), 7.47 (1H, dt, J=1.5, 7.6 Hz), 8.20-8.23 (1H, m).

The compounds shown by the following formula (V-a) were produced according to Reference Example 2.

### 2-ethyl-6-methylphenylboronic acid

Melting Point of 90 to 91° C.

¹H-NMR (CDCl₃) δ ppm: 1.22 (3H, t, J=7.6 Hz), 2.35 (3H, s), 2.64 (2H, q, J=7.6 Hz), 4.0-5.5 (2H, br.), 6.98 (1H, d, J=7.7 Hz), 7.01 (1H, d, J=7.7 Hz), 7.18 (1H, t, J=7.7 Hz).

### 2,6-diethyl-4-methylphenylboronic acid

Melting Point of 111 to 113° C.

¹H-NMR (CDCl₃) δ ppm: 1.23 (6H, t, J=7.7 Hz), 2.31 (3H, s), 2.63 (4H, q, J=7.7 Hz), 4.0-5.0 (2H, br.), 6.88 (2H, s).

### 5-(4-chlorophenyl)-2-methylphenylboronic acid

¹H-NMR (CDCl₃) δ ppm: 2.89 (3H, s), 7.34-7.46 (4H, m), 7.58 (2H, d, J=8.5 Hz), 7.64-7.70 (1H, m).

Representative production examples of the compounds shown by the formula (VI) are shown in Reference Example 3.

### Reference Example 3

1.5 g of potassium carbonate was added to 35 ml of an acetonitrile solution of 2.0 g of 2-(methylhydrazono) ethyl propanoate. To this mixture was added dropwise 10 ml of an acetonitrile solution of 2.6 g of 2,6-diethyl-4-methylphenylacetyl chloride under cooling with ice over about 20 minutes. This mixture was stirred at room temperature for 3.5 hours. The reaction mixture was concentrated. 20 mL of ice water was added to the residue, and the mixture was extracted with ethyl acetate (20 mL×3). The organic layer was washed with saturated saline (20 mL×2), dried with anhydrous magnesium sulfate, and concentrated. The residue was applied to a basic alumina column chromatography (ethyl acetate:hexane=1:3), to give 1.9 g of 2-[2-(2,6-diethyl-4-methylphenylacetyl)-2-methylhydrazono]ethyl propanoate [compound (VI-2)] as a white crystal.

The compounds of the formula (VI) produced according to Reference Example 3 are shown in Table 10 with the compound (VI-2).

The compounds shown by the formula (VI):

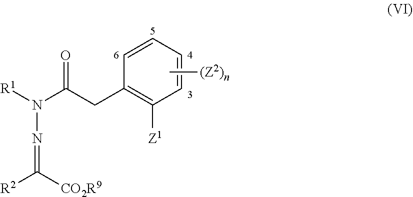

TABLE 10

| | | | | | | Melting |
|---|---|---|---|---|---|---|
| | | | | | | Point/ |
| No. | R¹ | R² | Z¹ | (Z²)ₙ | R⁹ | ° C. |
| VI-1 | Me | Me | Me | 4-Me, 6-Me | Et | 90-91 |
| VI-2 | Me | Me | Et | 4-Me, 6-Et | Et | 73-76 |
| VI-3 | Me | Me | Et | — | Et | * |
| VI-4 | Me | Me | Et | 4-Me | Et | * |
| VI-5 | Me | Me | Et | 4-Et, 6-Et | Et | 63-66 |
| VI-6 | Me | Me | Et | 4-Me, 6-Me | Et | * |
| VI-7 | Me | Et | Et | 4-Me, 6-Et | Et | * |
| VI-8 | Me | Et | Et | 4-Me | Et | * |
| VI-9 | Me | Et | Et | 4-Me, 6-Me | Et | * |
| VI-10 | Me | Et | Me | 4-Me, 6-Me | Et | * |
| VI-11 | Me | i-Pr | Et | 4-Me, 6-Et | Et | * |
| VI-12 | Me | Pr | Et | 4-Me, 6-Et | Et | * |
| VI-13 | Me | Et | Et | 4-Et, 6-Et | Et | * |
| VI-14 | Me | Pr | Et | 4-Et, 6-Et | Et | * |
| VI-15 | Me | Me | Et | 4-Et | Et | * |
| VI-16 | Me | Et | Et | 4-Et | Et | * |
| VI-17 | Me | Me | Et | 4-Et, 6-Me | Et | * |
| VI-18 | Me | Me | Et | 6-Et | Et | * |
| VI-19 | Me | Me | Me | 4-Me, 6-Me | Me | * |
| VI-20 | Me | Me | Et | 4-Et, 6-Et | Me | * |
| VI-34 | Et | Me | Et | 4-Et, 6-Et | Et | * |

Regarding the compounds marked with asterisk (*) in the column of melting point of Table 10, ¹H NMR data are shown below.

### Compound (VI-3):

¹H-NMR (CDCl₃) δ ppm: 1.19 (3H, t, J=7.6 Hz), 1.37 (3H, t, J=7.2 Hz), 2.20 (3H, br.s), 2.67 (2H, q, J=7.7 Hz), 3.37 (3H, br.s), 4.03 (2H, br.s), 4.33 (2H, q, J=7.0 Hz), 7.06-7.30 (4H, m).

Compound (VI-4):

¹H-NMR (CDCl₃) δ ppm: 1.18 (3H, t, J=7.6 Hz), 1.37 (3H, t, J=7.2 Hz), 2.20 (3H, br.s), 2.30 (3H, s), 2.63 (2H, q, J=7.7 Hz), 3.36 (3H, br.s), 3.99 (2H, br.s), 4.33 (2H, q, J=7.1 Hz), 6.93 (1H, br.d, J=7.1 Hz), 7.00 (1H, br.s), 7.12 (1H, br.d, J=7.8 Hz).

### Compound (VI-6):

¹H-NMR (CDCl₃) δ ppm: 1.16 (3H, t, J=7.7 Hz), 1.36 (3H, t, J=7.2 Hz), 2.22 (3H, s), 2.27 (3H, s), 2.30 (3H, br.s), 2.56 (2H, q, J=7.7 Hz), 3.39 (3H, br.s), 4.02 (2H, br.s), 4.32 (2H, q, J=7.1 Hz), 6.86 (2H, br.s).

Compound (VI-7) (mixture of E/Z isomers):

¹H-NMR (CDCl₃) δ ppm: 1.13-1.25 (9H, m), 1.31-1.41 (3H, m), 2.29 (3H, s), 2.50-2.81 (6H, m), 3.23, 3.43 (3H, each br.s), 4.05 (2H, br.s), 4.27-4.39 (2H, m), 6.89 (2H, s).

Compound (VI-8) (mixture of E/Z isomers):

¹H-NMR (CDCl₃) δ ppm: 1.06-1.22 (6H, m), 1.31-1.40 (3H, m), 2.30, 2.31 (3H, each s), 2.50-2.70 (4H, m), 3.22, 3.38 (3H, each s), 4.00 (2H, br.s), 4.27-4.37 (2H, m), 6.90-6.98 (1H, m), 6.98-7.02 (1H, m), 7.02-7.14 (1H, m).

Compound (VI-9) (mixture of E/Z isomers):

¹H-NMR (CDCl₃) δ ppm: 1.12-1.25 (6H, m), 1.31-1.41 (3H, m), 2.22 (3H, s), 2.27 (3H, s), 2.50-2.81 (4H, m), 3.23, 3.43 (3H, each br.s), 4.02 (2H, br.s), 4.26-4.37 (2H, m), 6.87 (2H, br.s).

Compound (VI-10) (mixture of E/Z isomers):

¹H-NMR (CDCl₃) δ ppm: 1.16-1.24 (3H, m), 1.32-1.40 (3H, m), 2.22 (6H, s), 2.25 (3H, s), 2.55-2.80 (2H, m), 3.23, 3.43 (3H, each br.s), 4.00 (2H, br.s), 4.27-4.38 (2H, m), 6.85 (2H, s).

### Compound (VI-11):

¹H-NMR (CDCl₃) δ ppm: 1.18 (6H, t, J=7.6 Hz), 1.24 (6H, d, J=6.8 Hz), 1.37 (3H, t, J=7.1 Hz), 2.29 (3H, s), 2.55 (4H, q, J=7.6 Hz), 2.85 (1H, septet, J=6.8 Hz), 3.22 (3H, s), 4.04 (2H, s), 4.34 (2H, q, J=7.2 Hz), 6.88 (2H, s).

Compound (VI-12) (mixture of E/Z isomers):

¹H-NMR (CDCl₃) δ ppm: 1.01 (3H, t, J=7.4 Hz), 1.17 (6H, t, J=7.6 Hz), 1.31-1.40 (3H, m), 1.57-1.74 (2H, m), 2.30 (3H, s), 2.50-2.76 (6H, m), 3.22, 3.42 (3H, each s), 4.03, 4.05 (2H, each br.s), 4.26-4.36 (2H, m), 6.89 (2H, s).

Compound (VI-13) (mixture of E/Z isomers):

¹H-NMR (CDCl₃) δ ppm: 1.13-1.28 (12H, m), 1.30-1.40 (3H, m), 2.50-2.80 (8H, m), 3.23, 3.44 (3H, each s), 4.06 (2H, br.s), 4.28-4.39 (2H, m), 6.91 (2H, s).

Compound (VI-14) (mixture of E/Z isomers):

¹H-NMR (CDCl₃) δ ppm: 1.01 (3H, br.t, J=7.2 Hz), 1.13-1.26 (9H, m), 1.30-1.40 (3H, m), 1.56-1.73 (2H, m), 2.50-2.76 (8H, m), 3.22, 3.42 (3H, each s), 4.03, 4.06 (2H, each br.s), 4.26-4.37 (2H, m), 6.91 (2H, s).

### Compound (VI-15):

¹H-NMR (CDCl₃) δ ppm: 1.15-1.25 (6H, m), 1.37 (3H, t, J=7.2 Hz), 2.20 (3H, br.s), 2.55-2.70 (4H, m), 3.36 (3H, br.s), 3.99 (2H, br.s), 4.33 (2H, q, J=7.1 Hz), 6.96 (1H, br.d, J=7.3 Hz), 7.02 (1H, br.s), 7.15 (1H, br.d, J=7.8 Hz).

Compound (VI-16) (mixture of E/Z isomers):

¹H-NMR (CDCl₃) δ ppm: 1.05-1.25 (9H, m), 1.32-1.40 (3H, m), 2.50-2.69 (6H, m), 3.22, 3.38 (3H, each s), 4.00 (2H, br.s), 4.26-4.36 (2H, m), 6.93-7.00 (1H, m), 7.00-7.04 (1H, m), 7.06-7.18 (1H, m).

### Compound (VI-17):

¹H-NMR (CDCl₃) δ ppm: 1.17 (3H, t, J=7.6 Hz), 1.22 (3H, t, J=7.6 Hz), 1.36 (3H, t, J=7.1 Hz), 2.24 (3H, s), 2.30 (3H, br.s), 2.58 (4H, q, J=7.6 Hz), 3.40 (3H, br.s), 4.03 (2H, br.s), 4.32 (2H, q, J=7.2 Hz), 6.89 (2H, s).

### Compound (VI-18):

¹H-NMR (CDCl₃) δ ppm: 1.19 (6H, t, J=7.6 Hz), 1.36 (3H, t, J=7.2 Hz), 2.32 (3H, br.s), 2.60 (4H, q, J=7.7 Hz), 3.40 (3H, br.s), 4.09 (2H, br.s), 4.33 (2H, q, J=7.2 Hz), 7.07 (2H, d, J=7.6 Hz), 7.18 (1H, t, J=7.6 Hz).

### Compound (VI-19):

¹H-NMR (CDCl₃) δ ppm: 2.21 (6H, s), 2.25 (3H, s), 2.29 (3H, br.s), 3.39 (3H, br.s), 3.88 (3H, s), 3.99 (2H, br.s), 6.85 (2H, s).

### Compound (VI-20):

¹H-NMR (CDCl₃) δ ppm: 1.18 (6H, t, J=7.6 Hz), 1.23 (3H, t, J=7.6 Hz), 2.32 (3H, br.s), 2.57 (4H, q, J=7.6 Hz), 2.60 (2H, q, J=7.6 Hz), 3.40 (3H, br.s), 3.88 (3H, s), 4.04 (2H, br.s), 6.90 (2H, s).

### Compound (VI-34):

¹H-NMR (CDCl₃) δ ppm: 1.17-1.22 (9H, m), 1.23 (3H, t, J=8 Hz), 1.35 (3H, t, J=7.2 Hz), 2.29 (3H, br.s), 2.54-2.63 (6H, m), 3.95 (2H, q, J=7.2 Hz), 3.99 (2H, br.s), 4.32 (2H, q, J=7.2 Hz), 6.91 (2H, s).

Representative production examples of the compounds shown by the formula (VI) are shown in Reference Example 4.

### Reference Example 4

0.68 g of potassium carbonate was added to 20 ml of an acetonitrile solution of 1.1 g of ethyl 2-(methylhydrazono) propanoate. To this mixture was added dropwise 8 ml of an acetonitrile solution of 1.26 g of 4-chloro-2,6-diethylphenylacetyl chloride under cooling with ice over about 10 minutes. This mixture was stirred at room temperature for 3 hours. The reaction mixture was concentrated. 20 mL of ice water was added to the resulting residue, and the mixture was extracted with ethyl acetate (20 mL, 10 mL×2). The organic layer was washed with saturated saline (20 mL×2), dried with anhydrous magnesium sulfate, and concentrated. The residue was applied to a basic alumina column chromatography (ethyl acetate:hexane=1:6), to give 1.2 g of a light yellow solid. The solid was washed with cold hexane and dried, to give 0.79 g of 2-[2-(4-chloro-2,6-diethylphenylacetyl)-2-methylhydrazono]ethyl propanoate [compound (VI-22)] as a white powder.

The compounds of the formula (VI) produced according to Reference Example 4 are shown in Table 11 with the compound (VI-22).

The compounds shown by the formula (VI):

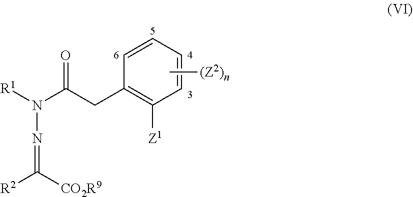

TABLE 11

| | | | | | | Melting |
|---|---|---|---|---|---|---|
| No. | R¹ | R² | Z¹ | (Z²)ₙ | R⁹ | Point/° C. |
| VI-21 | Me | Me | Et | 4-Ph, 6-Et | Et | 75-78 |
| VI-22 | Me | Me | Et | 4-Cl, 6-Et | Et | 85-89 |
| VI-23 | Me | Me | Et | 4-OMe, 6-Et | Et | * |
| VI-24 | Me | Me | Et | 4-Br, 6-Et | Et | 80-84 |
| VI-25 | Me | Me | Et | 4-CN, 6-Et | Et | * |
| VI-26 | Me | Me | Et | 4-CF₃, 6-Et | Et | 90-91 |
| VI-27 | Me | Me | Et | 4-OCF₃, 6-Et | Et | 75-76 |
| VI-28 | Me | Me | Et | 4-(c-Pr), 6-Et | Et | * |
| VI-29 | Me | Me | Et | 4-(4-Me—Ph), 6-Et | Et | * |
| VI-30 | Me | Me | Me | 5-(4-Cl—Ph) | Et | * |
| VI-31 | Me | Me | Me | 4-Me, 6-Br | Et | * |
| VI-32 | Me | Me | Me | 4-Me, 6-OMe | Et | * |

Regarding the compounds marked with asterisk (*) in the column of melting point of Table 11, ¹H NMR data are shown below.

### Compound (VI-23):

¹H-NMR (CDCl₃) δ ppm: 1.18 (6H, t, J=7.6 Hz), 1.36 (3H, t, J=7.1 Hz), 2.31 (3H, br.s), 2.57 (4H, q, J=7.4 Hz), 3.40 (3H, br.s), 3.79 (3H, s), 4.01 (2H, br.s), 4.33 (2H, q, J=7.1 Hz), 6.64 (2H, s).

### Compound (VI-25):

¹H-NMR (CDCl₃) δ ppm: 1.20 (6H, t, J=7.6 Hz), 1.37 (3H, t, J=7.1 Hz), 2.34 (3H, s), 2.61 (4H, q, J=7.5 Hz), 3.41 (3H, s), 4.12 (2H, s), 4.34 (2H, q, J=7.1 Hz), 7.36 (2H, s).

### Compound (VI-28):

¹H-NMR (CDCl₃) δ ppm: 0.62-0.73 (2H, m), 0.85-0.95 (2H, m), 1.17 (6H, t, J=7.6 Hz), 1.35 (3H, t, J=7.1 Hz), 1.79-1.90 (1H, m), 2.31 (3H, br.s), 2.55 (4H, q, J=7.6 Hz), 3.39 (3H, br.s), 4.04 (2H, br.s), 4.32 (2H, q, J=7.1 Hz), 6.78 (2H, s).

### Compound (VI-29):

¹H-NMR (CDCl₃) δ ppm: 1.23 (6H, t, J=7.6 Hz), 1.36 (3H, t, J=7.2 Hz), 2.33 (3H, br.s), 2.39 (3H, s), 2.65 (4H, q, J=7.6 Hz), 3.42 (3H, br.s), 4.12 (2H, br.s), 4.33 (2H, q, J=7.1 Hz), 7.23 (2H, d, J=8.0 Hz), 7.28 (2H, s), 7.49 (2H, d, J=8.3 Hz).

### Compound (VI-30):

¹H-NMR (CDCl₃) δ ppm: 10.33 (3H, t, J=7.2 Hz), 2.21 (3H, s), 2.35 (3H, s), 3.37 (3H, s), 4.06 (2H, br.s), 4.28 (2H, q, J=7.1 Hz), 7.22 (1H, d, J=7.8 Hz), 7.30-7.40 (3H, m), 7.43 (1H, br.s), 7.47 (2H, d, J=8.3 Hz).

### Compound (VI-31):

¹H-NMR (CDCl₃) δ ppm: 1.37 (3H, t, J=7.1 Hz), 2.20-2.39 (9H, m), 3.40 (3H, br.s), 4.16 (2H, br.s), 4.33 (2H, q, J=7.2 Hz), 6.94 (1H, br.s), 7.25 (1H, br.s).

### Compound (VI-32):

¹H-NMR (CDCl₃) δ ppm: 1.36 (3H, t, J=7.1 Hz), 2.15-2.35 (9H, m), 3.37 (3H, br.s), 3.75 (3H, s), 3.95 (2H, br.s), 4.32 (2H, q, J=7.1 Hz), 6.55 (1H, br.s), 6.63 (1H, br.s).

### Reference Example 5

4.96 g of pyridine was added to 80 ml of a toluene solution of 9.0 g of 2-(methylhydrazono) ethyl propanoate. To this mixture was added dropwise 40 ml of a toluene solution of 14.8 g of 5-bromo-2-methylphenylacetyl chloride under cooling with ice over about 50 minutes. This mixture was stirred at room temperature for 2 hours. Ice water (200 mL) was added to the reaction mixture, and the mixture was separated, then the aqueous layer was extracted with toluene (100 mL). The organic layer was washed with saturated saline (100 mL×2), dried with anhydrous magnesium sulfate, and concentrated. The residue was applied to a silica gel column chromatography (ethyl acetate:hexane=1:2), to give 14.3 g of 2-[2-(5-bromo-2-methylphenylacetyl)-2-methylhydrazono]ethyl propanoate [compound (VI-33)] as a light yellow oily substance.

¹H-NMR (CDCl₃) δ ppm: 1.39 (3H, t, J=7.3 Hz), 2.23 (3H, s), 2.27 (3H, s), 3.37 (3H, s), 3.39 (2H, s), 4.35 (2H, q, J=7.3 Hz), 7.02 (1H, d, J=8.2 Hz), 7.23-7.26 (1H, br, s), 7.41 (1H, br, s).

### Reference Example 6

To a mixture of 24.68 g of 2-(methylhydrazono) ethyl propanoate, 15.70 g of pyridine and 120.0 g of toluene was added dropwise a mixture of 28.32 g of ethyl malonyl chloride and 80.0 g of toluene under cooling with ice. This mixture was stirred under cooling with ice for 2 hours. 80.0 g of water was added to this mixture, and the mixture was separated. The organic layer was washed with 80.0 g of water and dried with anhydrous magnesium sulfate, then concentrated. 42.74 g of 2-[N-(3-ethoxy-3-oxopropionyl)-N-methylhydrazono]ethyl propanoate was obtained as a brown oily substance (GC area value of 82.4%).

To a mixture of 14.01 g of potassium tert-butoxide and 217.0 g of tetrahydrofuran was added dropwise a mixture of 31.0 g of 2-[N-(3-ethoxy-3-oxopropionyl)-N-methylhydrazono]ethyl propanoate and 93.0 g of tetrahydrofuran at 35° C. This mixture was stirred at 35° C. for 1 hour. The reaction mixture was cooled to room temperature. 93.0 g of water and 62.0 g of n-hexane were added to the reaction mixture, and the mixture was separated. 20.59 g of concentrated hydrochloric acid and 93.0 g of tert-butylmethylether were added, and the mixture was separated. The organic layer was dried with anhydrous magnesium sulfate and then concentrated, to give 17.13 g of ethyl 2,3-dihydro-5-hydroxy-2,6-dimethyl-3-oxo-4-pyridazine carbonate.

¹H-NMR (CDCl₃) δ ppm: 1.46 (3H, t, J=7.1 Hz), 2.30 (3H, s), 3.70 (3H, s), 4.48 (2H, q, J=7.1 Hz), 13.09 (1H, s).

A mixture of 15.0 g of ethyl 2,3-dihydro-5-hydroxy-2,6-dimethyl-3-oxo-4-pyridazine carbonate and 45.0 g of 12% hydrochloric acid was stirred at 70° C. for 4 hours. The reaction mixture was cooled to room temperature. The crystal precipitated in the reaction mixture was collected by filtration. This crystal was sequentially washed with water and methanol, and dried, to give 7.65 g of 2,3-dihydro-5-hydroxy-2,6-dimethyl-3-oxo-4-pyridazine carbonate as a white solid.

¹H-NMR (CDCl₃) δ ppm: 2.38 (3H, s), 3.80 (3H, s), 13.06 (1H, s), 14.81 (1H, br.s).

To a mixture of 2.00 g of 2,3-dihydro-5-hydroxy-2,6-dimethyl-3-oxo-4-pyridazine carbonate, 1.76 g of potassium carbonate and 6.00 g of water was added dropwise 1.86 g of bromine under cooling with ice. This mixture was stirred at room temperature for 2.5 hours. 2.33 g of concentrated hydrochloric acid was added dropwise to the reaction mixture. This mixture was filtered, to collect a solid. This solid was washed with 4.0 g of water and 4.0 g of n-hexane, and dried, to give 2.07 g of 4-bromo-5-hydroxy-2,6-dimethyl-3(2H)-pyridazinone as a white solid.

¹H-NMR (CDCl₃) δ ppm: 2.35 (3H, s), 3.78 (3H, s), 6.22 (1H, br.s).

To a mixture of 2.0 g of 4-bromo-5-hydroxy-2,6-dimethyl-3(2H)-pyridazinone, 1.51 g of potassium carbonate and 20.0 g of acetonitrile was added 1.27 g of dimethyl sulfate. This mixture was stirred at 50° C. for 12 hours. The reaction mixture was concentrated. Ethyl acetate and water were added to the residue, and the mixture was separated. The organic layer was washed with saturated saline, dried with magnesium sulfate, and concentrated, to give 2.01 g of 4-bromo-5-methoxy-2,6-dimethyl-3(2H)-pyridazinone as a light brown solid.

¹H-NMR (CDCl₃) δ ppm: 2.31 (3H, s), 3.77 (3H, s), 4.03 (3H, s).

### Reference Example 7

The same procedure as in Reference Example 1 was carried out, to give 4-(2,5-dimethylphenyl)-5-methoxy-2,6-dimethyl-3(2H)-pyridazinone [compound (II-15)] from 4-bromo-5-methoxy-2,6-dimethyl-3(2H)-pyridazinone and 2,5-dimethylphenylboronic acid.

The compounds of the formula (II) produced according to Reference Example 7 are shown in Table 12 with the compound (II-15).

The compounds shown by the formula (II):

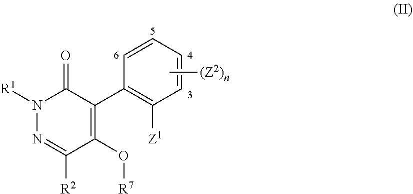

TABLE 12

| | | | | | | Melting |
|---|---|---|---|---|---|---|
| No. | R¹ | R² | Z¹ | (Z²)ₙ | R⁷ | Point/° C. |
| II-15 | Me | Me | Me | 5-Me | Me | * |
| II-16 | Me | Me | Me | 4-(4-Cl—Ph) | Me | * |
| II-17 | Me | Me | Et | 5-(4-Cl—Ph) | Me | * |
| II-19 | Me | Me | Me | 4-Me, 6-Me | Me | * |

Regarding the compounds marked with asterisk (*) in the column of melting point of Table 12, ¹H NMR data are shown below.

### Compound (II-15):

¹H-NMR (CDCl₃) δ ppm: 2.15 (3H, s), 2.28 (3H, s), 2.31 (3H, s), 3.35 (3H, s), 3.73 (3H, s), 6.95-7.31 (3H, m).

### Compound (II-16):

¹H-NMR (CDCl₃) δ ppm: 2.28 (3H, s), 2.30 (3H, s), 3.40 (3H, s), 3.75 (3H, s), 7.22-7.28 (1H, m), 7.38-7.48 (4H, m), 7.54 (2H, d, J=8.6 Hz).

### Compound (II-17):

¹H-NMR (CDCl₃) δ ppm: 1.21 (3H, t, J=7.8 Hz), 2.29 (3H, s), 3.40 (3H, s), 3.75 (3H, s), 7.33-7.43 (4H, m), 7.48-7.58 (3H, m).

### Compound (II-19):

¹H-NMR (CDCl₃) δ ppm: 2.09 (6H, s), 2.27 (3H, s), 2.29 (3H, s), 3.33 (3H, s), 3.73 (3H, s), 6.89 (2H, s).

### Reference Example 8

0.37 g of N-chlorosuccinimide was added to 5 ml of a N,N-dimethylformamide solution of 0.7 g of 5-methoxy-2-methyl-4-(2,4,6-trimethylphenyl)-3(2H)-pyridazinone [compound (II-10)]. This mixture was stirred at 60° C. for 3 hours and at 100° C. for 3 hours. The reaction mixture was cooled. The reaction mixture was poured into 50 ml of ice water. The produced solid was collected by filtration. This solid was washed with water and dried, to give 0.72 g of 4-(3-chloro-2,4,6-trimethylphenyl)-5-methoxy-2-methyl-3(2H)-pyridazinone [compound (II-18)] as a white crystal.

¹H-NMR (CDCl₃) δ ppm: 1.59 (3H, s), 2.02 (3H, s), 2.19 (3H, s), 2.36 (3H, s), 3.86 (6H, s), 7.01 (1H, s), 7.86 (1H, s).

The same procedure as in Reference Example 8 was carried out, to give 4-(3-chloro-2,4,6-trimethylphenyl)-5-methoxy-2,6-dimethyl-3(2H)-pyridazinone [compound (II-20)].

¹H-NMR (CDCl₃) δ ppm: 2.06 (3H, s), 2.19 (3H, s), 2.28 (3H, s), 2.37 (3H, s), 3.35 (3H, s), 3.73 (3H, s), 6.99 (1H, s).

Hereinbelow, preparation examples are shown. Incidentally, in the following examples, a part represents a part by weight. In addition, the compounds I-a-1 to I-a-75, and I-b-1 to I-b-63 correspond to the numbers of the compounds described in the production examples.

### Preparation Example 1

5 parts of any of the compounds I-a-1 to I-a-75, and I-b-1 to I-b-63, 2.5 parts of metconazole, 14 parts of polyoxyethylenestyrylphenyl ether, 6 parts of calcium dodecylbenzenesulfonate, and 72.5 parts of xylene are mixed, to give each preparation.

### Preparation Example 2

10 parts of any of the compounds I-a-1 to I-a-75, and I-b-1 to I-b-63, 5 parts of ethaboxam, 35 parts of a mixture of white carbon and polyoxyethylene alkyl ether sulfate ammonium salt (weight ratio 1:1), and 50 parts of water are mixed, and this mixture is finely-pulverized by wet grinding method, to give each preparation.

### Preparation Example 3

15 parts of any of the compounds I-a-1 to I-a-75, and I-b-1 to I-b-63, 5 parts of 2-[2-(2,5-dimethylphenoxymethyl)phenyl]-2-methoxy-N-methylacetamide and 1.5 parts of sorbitan trioleate are added to a mixture of 2 parts of polyvinyl alcohol and 26.5 parts of water, and this mixture is finely-pulverized by wet grinding method. Thereto are added a mixture of 0.05 parts of xanthan gum, 0.1 parts of aluminum magnesium silicate and 39.85 parts of an aqueous solution and 10 parts of propylene glycol, and stirred, to give each preparation.

### Preparation Example 4

25 parts of any of the compounds I-a-1 to I-a-75, and I-b-1 to I-b-63, 5 parts of tolclofos-methyl and 1.5 parts of sorbitan trioleate are added to a mixture of 2 parts of polyvinyl alcohol and 26.5 parts of an aqueous solution, and this mixture is finely-pulverized by wet grinding method. Thereto are added a mixture of 0.05 parts of xanthan gum, 0.1 parts of aluminum magnesium silicate and 29.85 parts of water and 10 parts of propylene glycol, and stirred, to give each preparation.

### Preparation Example 5

45 parts of any of the compounds I-a-1 to I-a-75, and I-b-1 to I-b-63, 5 parts of ipconazole, 5 parts of propylene glycol, 5 parts of Soprophor (trademark) FLK (tradename, manufactured by Rhodia Nicca Ltd.), 0.2 parts of ANTIFOAM C (trademark) emulsion (tradename, manufactured by Dow Corning Corporation), 0.3 parts of Proxel (trademark) GXL (tradename, manufactured by Arch Chemicals, Inc.) and 39.5 parts of ion exchanged water are mixed. 100 parts of this mixture and 150 parts of glass beads (φ=1 mm) are mixed, and stirred for 2 hours while cooling. The glass beads are removed by filtration, to give each preparation.

### Preparation Example 6

50 parts of any of the compounds I-a-1 to I-a-75, and I-b-1 to I-b-63, 38.5 parts of NN kaolin clay (manufactured by TAKEHARA KAGAKU KOGYO CO., LTD), 10 parts of Morwet D425 (tradename, manufactured by Akzo Nobel) and 1.5 parts of Morwet EFW (tradename, manufactured by DESOTO) are mixed. This mixture is pulverized with a jet mill, to give each preparation.

### Preparation Example 7

5 parts of any of the compounds I-a-1 to I-a-75, and I-b-1 to I-b-63, 1 part of synthetic hydrous silicon oxide, 2 parts of calcium lignin sulfonate, 30 parts of bentonite, and 62 parts of kaolin clay are mixed. Water was added to this mixture and kneaded together, then granulated and dried, to give each preparation.

### Preparation Example 8

10 parts of any of the compounds I-a-1 to I-a-75, and I-b-1 to I-b-63, 3 parts of calcium lignin sulfonate, 2 parts of sodium lauryl sulfate, and 85 parts of synthetic hydrous silicon oxide are pulverized and mixed, to give each preparation.

### Preparation Example 9

2 parts of any of the compounds I-a-1 to I-a-75, and I-b-1 to I-b-63, 88 parts of kaolin clay, and 10 parts of talc are pulverized and mixed, to give each preparation.

### Preparation Example 10

5 parts of any of the compounds I-a-1 to I-a-75, and I-b-1 to I-b-63, 14 parts of polyoxyethylenestyrylphenyl ether, 6 parts of calcium dodecylbenzenesulfonate, and 75 parts of xylene are mixed, to give each preparation.

### Preparation Example 11

5 parts of any of the compounds I-a-1 to I-a-75, and I-b-1 to I-b-63 and 1.5 parts of sorbitan trioleate are added to a mixture of 2 parts of polyvinyl alcohol and 28 parts of water, and this mixture is finely-pulverized by wet grinding method. Thereto are added a mixture of 0.05 parts of xanthan gum, 0.1 parts of aluminum magnesium silicate and 48.35 parts of water, and mixed, to give each preparation.

### Preparation Example 12

1 part of any of the compounds I-a-1 to I-a-75, and I-b-1 to I-b-63, 1 part of synthetic hydrous silicon oxide, 2 parts of calcium lignin sulfonate, 30 parts of bentonite, and 66 parts of kaolin clay are mixed. Water was added to this mixture and kneaded together, then granulated and dried, to give each preparation.

### Preparation Example 13

40 parts of any of the compounds I-a-1 to I-a-75, and I-b-1 to I-b-63, 3 parts of calcium lignin sulfonate, 2 parts of sodium lauryl sulfate, and 55 parts of synthetic hydrous silicon oxide are pulverized and mixed, to give each preparation.

### Preparation Example 14

10 parts of any of the compounds I-a-1 to I-a-75, and I-b-1 to I-b-63, 35 parts of a mixture of white carbon and polyoxyethylene alkyl ether sulfate ammonium salt (weight ratio 1:1), and 55 parts of water are mixed, and this mixture is finely-pulverized by wet grinding method, to give each preparation.

Next, seed treatment examples are shown.

### Seed Treatment Example 1

500 ml of the preparation prepared according to Preparation Example 1 is used for smear treatment per 100 kg of sorghum seeds using a seed treater (manufactured by Hans-Ulrich Hege GmbH) so as to obtain treated seeds.

### Seed Treatment Example 2

50 ml of the preparation prepared according to Preparation Example 2 is used for smear treatment per 10 kg of rapeseeds using a seed treater (manufactured by Hans-Ulrich Hege GmbH) so as to obtain treated seeds.

### Seed Treatment Example 3

40 ml of the preparation prepared according to Preparation Example 3 is used for smear treatment per 10 kg of corn seeds using a seed treater (manufactured by Hans-Ulrich Hege GmbH) so as to obtain treated seeds.

### Seed Treatment Example 4

5 parts of the preparation prepared according to Preparation Example 4 and 40 parts of water are mixed. 60 ml of the mixture is used for smear treatment per 10 kg of rice seeds using a seed treater (manufactured by Hans-Ulrich Hege GmbH) so as to obtain treated seeds.

### Seed Treatment Example 5

5 parts of the preparation prepared according to Preparation Example 4 and 35 parts of water are mixed. 70 ml of the mixture is used for smear treatment per 10 kg of potato tuber piece using a seed treater (manufactured by Hans-Ulrich Hege GmbH) so as to obtain treated seeds.

### Seed Treatment Example 6

500 ml of the preparation prepared according to Preparation Example 5 is used for smear treatment per 100 kg of sugar beet seeds using a seed treater (manufactured by Hans-Ulrich Hege GmbH) so as to obtain treated seeds.

### Seed Treatment Example 7

50 ml of the preparation prepared according to Preparation Example 3 is used for smear treatment per 10 kg of soybean seeds using a seed treater (manufactured by Hans-Ulrich Hege GmbH) so as to obtain treated seeds.

### Seed Treatment Example 8

50 ml of the preparation prepared according to Preparation Example 2 is used for smear treatment per 10 kg of wheat seeds using a seed treater (manufactured by Hans-Ulrich Hege GmbH) so as to obtain treated seeds.

### Seed Treatment Example 9

50 g of the preparation prepared according to Preparation Example 6 is used for powder coating treatment per 10 kg of corn seeds so as to obtain treated seeds.

### Seed Treatment Example 10

40 g of the preparation prepared according to Preparation Example 9 is used for powder coating treatment per 10 kg of cotton seeds so as to obtain treated seeds.

Next, it is shown as test examples that the control agent of the present invention has an effect for the control of arthropod pests.

### Test Example 1

The preparation of the present composition obtained by Preparation Example 14 was diluted with water so as to have a concentration of active ingredient of 500 ppm, to prepare a drug solution for test.

The drug solution for test was sprayed at a rate of 20 mL/cup on Brassicae oleracea at the 3 leaf stage planted in a polyethylene cup. After the drug solution dried, leaves and stems were cut out and placed in a 50 mL cup. 5 second-stage larvae of Plutella xylostella were released there, and the lid was closed. This cup was stored at 25° C., and the number of surviving insects was counted after 5 days, and the mortality of the insects was calculated by the following equation.

Mortality of Insects (%)=(Number of Dead Insects/Number of Test Insects)×100

As a result, the areas treated with the spray solutions for test of the compounds I-a-40, I-a-46, I-b-48, I-a-58, I-a-59, I-a-61, I-a-64, I-b-51, I-b-52, I-b-53, I-b-54, I-b-55, I-b-56, I-b-57, I-b-58, I-b-59 and I-b-60 showed a mortality of the insects of 80% or more.

### Test Example 2

The preparation of the present composition obtained by Preparation Example 14 was diluted with water so as to have a concentration of active ingredient of 500 ppm, to prepare a drug solution for test.

SilkMate 2S (Nosan Corporation) sliced at a thickness of 2 mm was placed in the bottom of a polyethylene cup, and 1 mL of the above drug solution for test was added dropwise. After the drug solution dried, 30 first-instar larvae of Adoxophyes orana were released, and the lid was closed. This cup was stored at 25° C., and the number of surviving insects was counted after 7 days and evaluated by the following insecticidal index.

Insecticidal Index) 4: mortality 100%, 3: 80 to 99%, 2: 60 to 79%, 1: 30 to 59%, 0: 0 to 29%

As a result, the areas treated with the spray solutions for test of the compounds I-a-23, I-a-40, I-a-59, I-b-51, I-b-52, I-b-53, I-b-54, I-b-55, I-b-56, I-b-57, I-b-58I-b-59, I-b-61 and I-b-62 showed an index of 3 or more.

### Test Example 3

The preparation of the present composition obtained by Preparation Example 14 was diluted with water so as to have a concentration of active ingredient of 500 ppm, to prepare a drug solution for test.

10 mL of the above drug solution for test was sprayed on rice seedling at the second leaf-expansion stage planted in a polyethylene cup. After the drug solution dried, 30 first-instar larvae of Nilaparvata lugens were released. This cup was stored in a greenhouse at 25° C. The number of Nilaparvata lugens parasitic on rice was investigated after 6 days, and the preventive value was calculated by the following equation.

Preventive Value (%)={1−(Cb×Tai)/(Cai×Tb)}×100

Here, the letters in the equation represent the following meaning.

Cb: Number of Insects in Untreated Area Before Treatment

Cai: Number of Insects in Untreated Area at Observation

Tb: Number of Insects in Treated Area Before Treatment

Tai: Number of Insects in Treated Area at Observation

As a result, the areas treated with the spray solutions for test of the compounds I-a-9, I-a-10, I-a-15, I-a-40, I-a-46, I-a-48, I-a-51, I-a-56, I-a-58, I-a-59, I-a-62, I-a-63, I-a-64, I-b-51, I-b-52, I-b-55, I-b-56, I-b-58 and I-b-61 showed a preventive value of 90% or more.

### Test Example 4

The preparation of the present composition obtained by Preparation Example 14 was diluted with water so as to have a concentration of active ingredient of 500 ppm, to prepare a drug solution for test.

About 30 Aphis gossypii were inoculated into cucumber seedling at the second true leaf-expansion stage planted in a polyethylene cup, and 10 mL of the above drug solution for test was sprayed at 1 day after inoculation.

The number of surviving insects of Aphis gossypii parasitic on the leaves of cucumber was investigated at 5 days after spraying, and the preventive value was calculated by the following equation.

Preventive Value (%)={1−(Cb×Tai)/(Cai×Tb)}×100

Here, the letters in the equation represent the following meaning.

Cb: Number of Insects in Untreated Area Before Treatment

Cai: Number of Insects in Untreated Area at Observation

Tb: Number of Insects in Treated Area Before Treatment

Tai: Number of Insects in Treated Area at Observation

As a result, the areas treated with the spray solutions for test of the compounds I-a-3, I-a-10, I-a-40, I-a-46, I-a-48, I-a-56, I-a-58, I-a-59, I-a-63, I-a-64, I-b-51, I-b-52, I-b-55, I-b-56, I-b-58, I-b-61 and I-b-62 showed a preventive value of 90% or more.

### Test Example 5

The preparation of the present composition obtained by Preparation Example 14 was diluted with water so as to have a concentration of active ingredient of 500 ppm, to prepare a drug solution for test.

About 60 female adults of Tetranychus urticae were inoculated into Phaseolus vulgaris seedling at the primary leaf-expansion stage planted in a polyethylene cup, and 30 mL of the above drug solution for test was sprayed at 1 day after inoculation.

The number of surviving mites on the leaves of Phaseolus vulgaris was investigated at 8 days after spraying, and the preventive rate was calculated by the following equation.

Preventive Rate (%)={1−(Number of Surviving Mites in Treated Area)/(Number of Surviving Mites in Untreated Area)}×100

As a result, the areas treated with the spray solutions for test of the compounds I-a-13, I-a-40, I-a-46, I-a-48, I-a-58, I-a-59, I-a-64, I-b-55, I-b-56, I-b-57, I-b-58 and I-b-61 showed a preventive rate of 90% or more.

### Test Example 6

The preparation of the present composition obtained by Preparation Example 14 was diluted with water so as to have a concentration of active ingredient of 500 ppm, to prepare a drug solution for test.

A filter paper with a diameter of 5.5 cm was placed in the bottom of a polyethylene cup, then 0.7 mL of the above drug solution for test was added dropwise on the filter paper, and 30 mg of sucrose was uniformly added as a feed. 2 male adults of Blattalla germanica were released in the polyethylene cup, and the lid was closed. The number of dead insects was calculated after 6 days.

The mortality was calculated by the following equation and was evaluated by the indexes of 4: mortality 100%, 2:50%, and 0:0%.

Mortality (%)=(Number of Dead Insects/Number of Test Insects)×100

As a result, the area treated with the drug solution for test of the compound I-b-1 showed an index of 4.

### Test Example 7

The preparation of the present composition obtained by Preparation Example 14 was diluted with water so as to have a concentration of active ingredient of 500 ppm, to prepare a drug solution for test.

0.7 mL of the drug solution for test was diluted with ion exchanged water to 100 mL. 20 last instar larvae of Culex pipiens pallens were released in the diluted solution, and the number of dead insects was counted after 1 day.

The mortality was calculated by the following equation and was evaluated by the indexes of 4: mortality 91 to 100%, 2: 11 to 90%, and 0: 0 to 10%.

Mortality (%)=(Number of Dead Insects/Number of Test Insects)×100

As a result, the areas treated with the drug solutions for test of the compounds I-a-9, I-a-14, I-a-40, I-a-46, I-a-63, I-a-70 and I-b-48 showed an index of 4.

### Test Example 8

The preparation of the present composition obtained by Preparation Example 14 was diluted with water so as to have a concentration of active ingredient of 500 ppm, to prepare a drug solution for test.

The drug solution for test was sprayed at a rate of 20 mL/cup on Brassicae oleracea at the 5 to 6 leaf stages planted in a polyethylene cup. After the drug solution dried, 10 fourth-stage larvae of Spodoptera litura were released. Brassicae oleracea at the 5 to 6 leaf stage planted in this polyethylene cup was covered with an another polyethylene cup (volume of 400 mL). This was stored at 25° C., and the number of surviving insects was counted after 6 days, then the mortality of the insects was calculated by the following equation.

Mortality of Insects (%)=(Number of Dead Insects/Number of Test Insects)×100

As a result, the areas treated with the spray solutions for test of the compounds I-b-45, I-a-59, I-b-51, I-b-52, I-b-53, I-b-54, I-b-55, I-b-56, I-b-57, I-b-58, I-b-59 and I-b-61 showed a mortality of the insects of 80% or more.

### Test Example 9

The preparation of the present composition obtained by Preparation Example 14 was diluted with water so as to have a concentration of active ingredient of 500 ppm, to prepare a drug solution for test.

Adults of Bemisia tabaci were released to tomato at the third true leaf-expansion stage planted in a polyethylene cup and encouraged to lay eggs for about 24 hours. The tomato seedling was maintained in a greenhouse for 8 days, and the drug solution for test was sprayed at a rate of 10 ml/cup when larvae hatched from laid eggs, then the seedling was maintained in a greenhouse at 25° C. After 7 days, the number of surviving larvae on the leaves of tomato was investigated, and the preventive value was calculated by the following equation.

Preventive Value (%)={1−(Cb×Tai)/(Cai×Tb)}×100

Here, the letters in the equation represent the following meaning.

Cb: Number of Insects in Untreated Area Before Treatment

Cai: Number of Insects in Untreated Area at Observation

Tb: Number of Insects in Treated Area Before Treatment

Tai: Number of Insects in Treated Area at Observation

As a result, the areas treated with the spray solutions for test of the compounds I-a-40, I-a-45, I-a-46, I-a-58 and I-b-48 showed a preventive value of 90% or more.

### Test Example 10

The preparation of the present composition obtained by Preparation Example 14 was diluted with water so as to have a concentration of active ingredient of 500 ppm, to prepare a drug solution for test.

The drug solution for test was sprayed at a rate of 10 mL/cup on cucumber at the second true leaf-expansion stage planted in a polyethylene cup. After the drug solution dried, the first true leaves were cut out and placed in a polyethylene cup, then 20 larvae of Frankliniella occidentalis were released, and the lid was closed. After 7 days, the number of surviving larvae on the leaves of cucumber was investigated, and the preventive value was calculated by the following equation.

Preventive Value (%)={1−(Cb×Tai)/(Cai×Tb)}×100

Here, the letters in the equation represent the following meaning.

Cb: Number of Insects in Untreated Area Before Treatment

Cai: Number of Insects in Untreated Area at Observation

Tb: Number of Insects in Treated Area Before Treatment

Tai: Number of Insects in Treated Area at Observation

As a result, the area treated with the spray solution for test of the compound I-a-40 showed a preventive value of 90% or more.

### INDUSTRIAL APPLICABILITY

The present compound can be used for a use for controlling arthropod pests.

1. An agent for controlling arthropod pests comprising a pyridazinone compound shown by formula (I): 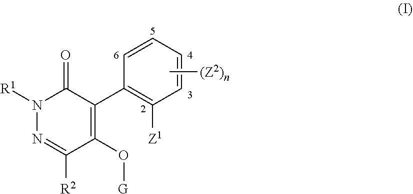
[wherein R¹ represents a C₁₋₆ alkyl group or a (C₁₋₆ alkyloxy)C₁₋₆ alkyl group,
R² represents hydrogen or a C₁₋₆ alkyl group,
G represents hydrogen, a group represented by: 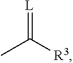
a group represented by: 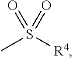
or a group represented by: 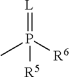
{wherein L represents oxygen or sulfur,
R³ represents a C₁₋₆ alkyl group, a C₃₋₈ cycloalkyl group, a C₂₋₆ alkenyl group, a C₂₋₆ alkynyl group, a C₆₋₁₀ aryl group, a (C₆₋₁₀ aryl)C₁₋₆ alkyl group, a C₁₋₆ alkyloxy group, a C₃₋₈ cycloalkyloxy group, a C₂₋₆ alkenyloxy group, a C₃₋₆ alkynyloxy group, a C₆₋₁₀ aryloxy group, a (C₆₋₁₀ aryl)C₁₋₆ alkyloxy group, a di(C₁₋₆ alkyl)amino group, a di(C₂₋₆ alkenyl)amino group, a (C₁₋₆ alkyl)(C₆₋₁₀ aryl)amino group or a 3- to 8-membered nitrogen-containing heterocyclic group,
R⁴ represents a C₁₋₆ alkyl group, a C₆₋₁₀ aryl group or a di(C₁₋₆ alkyl)amino group, and
R⁵ and R⁶ may be the same or different, and represent a C₁₋₆ alkyl group, a C₃₋₈ cycloalkyl group, a C₂₋₆ alkenyl group, a C₆₋₁₀ aryl group, a C₁₋₆ alkyloxy group, a C₃₋₈ cycloalkyloxy group, a C₆₋₁₀ aryloxy group, a (C₆₋₁₀ aryl)C₁₋₆ alkyloxy group, a C₁₋₆ alkylthio group or a di(C₁₋₆ alkyl)amino group,
wherein any group each represented by R³, R⁴, R⁵ and R⁶ may be substituted with halogen, and the C₃₋₈ cycloalkyl group, the C₆₋₁₀ aryl group, the aryl moiety of the (C₆₋₁₀ aryl)C₁₋₆ alkyl group, the C₃₋₈ cycloalkyloxy group, the C₆₋₁₀ aryloxy group, the aryl moiety of the (C₆₋₁₀ aryl)C₁₋₆ alkyloxy group, the aryl moiety of the (C₁₋₆ alkyl)(C₆₋₁₀ aryl)amino group and the 3- to 8-membered nitrogen-containing heterocyclic group may be substituted with a C₁₋₆ alkyl group,}
Z¹ represents a C₁₋₆ alkyl group,
Z² represents a C₁₋₆ alkyl group, a C₃₋₈ cycloalkyl group, a C₂₋₆ alkynyl group, a C₁₋₆ haloalkyl group, a C₆₋₁₀ aryl group, a 5- or 6-membered heteroaryl group, a C₁₋₆ alkyloxy group, a C₁₋₆ haloalkyloxy group, halogen, a cyano group or a nitro group, wherein the C₃₋₈ cycloalkyl group, the C₆₋₁₀ aryl group and the 5- or 6-membered heteroaryl group represented by Z² may be substituted with at least one group selected from the group consisting of halogens and C₁₋₆ alkyl groups, and
n represents 0, 1, 2, 3, or 4, and when n is 2, 3, or 4, each of Z² may be the same or different.]
and an inert carrier.

2. The agent for controlling arthropod pests according to claim 1, wherein, in the formula (I), n is 1, 2, or 3.

3. The agent for controlling arthropod pests according to claim 1, wherein, in the formula (I), n is 0, and Z¹ is a C₂₋₆ alkyl group.

4. The agent for controlling arthropod pests according to claim 1, wherein, in the formula (I), n is 1 or 2, and the binding position of Z² is a 4- and/or 6-positions on the benzene ring.

5. The agent for controlling arthropod pests according to claim 1, wherein, in the formula (I), Z¹ is a C₁₋₃ alkyl group, and Z² is a C₁₋₃ alkyl group, a C₃₋₆ cycloalkyl group, a C₂₋₃ alkynyl group, a C₁₋₃ alkyloxy group, halogen, a cyano group or a nitro group, or a phenyl group which may be substituted with at least one group selected from the group consisting of halogens and C₁₋₃ alkyl groups.

6. The agent for controlling arthropod pests according to claim 1, wherein, in the formula (I), Z¹ is a C₁₋₃ alkyl group, and Z² is a C₁₋₃ alkyl group.

7. The agent for controlling arthropod pests according to claim 1, wherein, in the formula (I), n is 1, and the binding position of Z² is a 5-position on the benzene ring.

8. The agent for controlling arthropod pests according to claim 1, wherein, in the formula (I), n is 1, the binding position of Z² is a 5-position on the benzene ring, Z¹ is a C₁₋₃ alkyl group, and Z² is a phenyl group which may be substituted with at least one group selected from the group consisting of halogens and C₁₋₃ alkyl groups.

9. The agent for controlling arthropod pests according to claim 1, wherein, in the formula (I), R¹ is a C₁₋₃ alkyl group or a (C₁₋₃ alkyloxy)C₁₋₃ alkyl group.

10. The agent for controlling arthropod pests according to claim 1, wherein, in the formula (I), R¹ is a methyl group.

11. The agent for controlling arthropod pests according to claim 1, wherein, in the foiniula (I), R² is hydrogen or a C₁₋₃ alkyl group.

12. The agent for controlling arthropod pests according to claim 1, wherein, in the formula (I), R² is hydrogen or a methyl group.

13. The agent for controlling arthropod pests according to claim 1, wherein, in the formula (I), G is hydrogen, a group represented by: 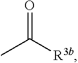
a group represented by: 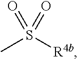
or a group represented by: 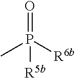
[wherein R^{3b} represents a C₁₋₆ alkyl group, a C₃₋₈ cycloalkyl group, a C₂₋₆ alkenyl group, a C₂₋₆ alkynyl group, a C₆₋₁₀ aryl group, a (C₆₋₁₀ aryl)C₁₋₆ alkyl group, a C₁₋₆ alkyloxy group, a C₃₋₈ cycloalkyloxy group, a C₂₋₆ alkenyloxy group, a C₆₋₁₀ aryloxy group, a (C₆₋₁₀ aryl)C₁₋₆ alkyloxy group or a di(C₁₋₆ alkyl)amino group,
R^{4b} represents a C₁₋₆ alkyl group or a C₆₋₁₀ aryl group, and
R^{5b} and R^{6b} may be the same or different, and represent a C₁₋₆ alkyl group, a C₁₋₆ alkyloxy group, a C₆₋₁₀ aryloxy group, or a C₁₋₆ alkylthio group,
wherein any group represented by any group each represented by R^{3b}, R^{4b}, R^{5b}, and R^{6b} may be substituted with halogen, and the C₃₋₈ cycloalkyl group, the C₆₋₁₀ aryl group, the aryl moiety of the (C₆₋₁₀ aryl)C₁₋₆ alkyl group, the C₃₋₈ cycloalkyloxy group, the C₆₋₁₀ aryloxy group, and the aryl moiety of the (C₆₋₁₀ aryl)C₁₋₆ alkyloxy group may be each substituted with a C₁₋₆ alkyl group].

14. The agent for controlling arthropod pests according to claim 1, wherein, in the formula (I), G is hydrogen, a group represented by: 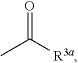
or a group represented by: 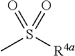
[wherein R^{3a} represents a C₁₋₆ alkyl group, a C₃₋₈ cycloalkyl group, a C₂₋₆ alkenyloxy group, a C₆₋₁₀ aryl group, a C₁₋₆ alkyloxy group or a di(C₁₋₆ alkyl)amino group, and
R^{4a} represents a C₁₋₆ alkyl group,
wherein any group represented by R^{3a} and R^{4a} may be substituted with halogen, and the C₃₋₈ cycloalkyl group and the C₆₋₁₀ aryl group may be both substituted with a C₁₋₆ alkyl group].

15. A method for controlling arthropod pests comprising a step of treating an arthropod pest or a place where an arthropod pest lives with an effective amount of the pyridazinone compound according to claim 1.

16. A use of the pyridazinone compound as defined in claim 1 for controlling arthropod pests.

17. The agent for controlling arthropod pests according to claim 2, wherein, in the formula (I), n is 1 or 2, and the binding position of Z² is a 4- and/or 6-positions on the benzene ring.
